(19) United States
(12) Patent Application Publication
(10) Pub. No.: US 2011/0229560 A1
(43) Pub. Date: Sep. 22, 2011

(54) NLRC5 AS A TARGET FOR IMMUNE THERAPY

(76) Inventors: Rong-Fu Wang, Houston, TX (US); Helen Y. Wang, Houston, TX (US); Jun Cui, Houston, TX (US); Liang Zhu, Houston, TX (US)

(21) Appl. No.: 13/122,838

(22) PCT Filed: Oct. 6, 2009
(86) PCT No.: PCT/US09/59711

§ 371 Date: Jun. 9, 2011

Related U.S. Application Data
(60) Provisional application No. 61/102,967, filed on Oct. 6, 2008.

(51) Int. Cl. A61K 9/127; A61K 31/7105; A61K 38/02; A61K 31/7088; A61K 35/12; A61P 35/00; A61P 37/04; A61P 31/00; A61P 31/04; A61P 31/12; A61P 31/06; A61P 31/18; A61P 31/16; A61P 31/10; A61P 31/14; A61P 31/22

(52) U.S. Cl. 424/450; 514/44 A; 514/19.3; 514/44 R; 424/93.71; 514/1.1

(57) ABSTRACT

The present invention concerns the enhancement of immune response to microbial infection and/or inflammation-associated disease through at least partial inhibition of NLRC5. The inhibition may be of any suitable means, although in particular cases it is via siRNA agents. In specific embodiments, a particular domain of NLRC5 is targeted by the siRNA.

### CROSS-REFERENCE TO RELATED APPLICATIONS

This application claims priority to U.S. Provisional Patent Application Ser. No. 61/102,967, filed Oct. 6, 2008, which is incorporated by reference herein in its entirety.

### STATEMENT REGARDING FEDERALLY SPONSORED RESEARCH OR DEVELOPMENT

This invention was made with government support under ROCA090327 awarded by the National Institutes of Health The government has certain rights in the invention.

### TECHNICAL FIELD

The present invention concerns at least the fields of cell biology, molecular biology, immunology, and medicine. In particular, the present invention relates to inhibition of NLRC5 to enhance immune responses.

### BACKGROUND OF THE INVENTION

The innate immune response, elicited through the detection of pathogen-associated molecular patterns (PAMPs), provides the first line of defense against invading microorganisms. PAMP recognition depends on several classes of pattern recognition receptors (PRRs), including Toll-like receptors (TLRs), NOD-like receptors (NLRs) and RIG-1-like receptors (RLRs) (Akira et al., 2006; Honda and Taniguchi, 2006; Inohara et al., 2005; Meylan et al., 2006; Ting and Davis, 2005). Activation of most TLRs leads to the recruitment of a common adaptor, MyD88, and in turn to a series of downstream signaling events that culminate in NF-κB activation (Akira et al., 2006; Chen, 2005; Hayden and Ghosh, 2008). By contrast, activation of RLRs (RIG-I and MDA5) by double- and single-stranded RNAs or certain viruses (Hornung et al., 2006; Kato et al., 2006; Pichlmair et al., 2006; Yoneyama et al., 2004) results in recruitment of the MAVS protein (mitochondrial antiviral signaling, also called VISA, IPS-1 and Cardif), which further activates the downstream signaling molecules TBK1/IKKi and IRF3 for type I interferon responses, as well as IKK molecules for NF-κB activation (Kawai et al., 2005; Meylan et al., 2005; Seth et al., 2005; Xu et al., 2005). Besides their roles in innate immunity and inflammation, TLR-mediated signaling pathways have been shown to play an important role in the control of regulatory T cell function (Liu et al., 2006; Pasare and Medzhitov, 2003; Peng et al., 2005; Peng et al., 2007; Sutmuller et al., 2006).

Because uncontrolled immune responses can be extremely harmful, even fatal, to the host (Liew et al., 2005), NF-κB activation and type I interferon signaling must be tightly regulated to maintain immune balance in the organism. Despite the importance of the IKK complex as a central transducer of signaling from various stimuli, leading to the activation of the NF-κB pathway, and of RLRs as critical receptors in type I interferon signaling (Chen, 2005; Hacker and Karin, 2006; Honda and Taniguchi, 2006), the molecular mechanisms responsible for IKK activation and RLR-mediated signaling remain poorly understood.

NLRs represent a large family of intracellular PRRs that are characterized by a conserved nucleotide-binding and oligomerization domain (NOD) and a leucine-rich repeat (LRR) region, and are involved in the activation of diverse signaling pathways (Akira et al., 2006; Inohara et al., 2005; Meylan et al., 2006; Ting and Davis, 2005). Several NLRs, such as NOD1, NOD2 and NALP3, have been extensively studied and shown to activate signaling pathways once they encounter relevant PAMPs (Akira et al., 2006; Inohara et al., 2005; Kobayashi et al., 2005; Meylan et al., 2006; Shaw et al., 2008; Ting and Davis, 2005). NALP3 inflammasome, for example, functions as a crucial component in the adjuvant effect of aluminium and asbestos (Dostert et al., 2008; Eisenbarth et al., 2008). More recently, NLRX1 (also known as NODS) was demonstrated to function as a mitochondrial protein that interacts with the mitochondrial adaptor MAVS to inhibit the RIG-1-mediated signaling pathway and triggers the generation of reactive oxygen species as well (Moore et al., 2008; Tattoli et al., 2008). These studies indicate that understanding the function and mechanisms of these innate immune receptors or regulators is useful in developing more effective strategies for the immunological treatment of inflammation-associated diseases (Karin et al., 2006; Wang et al., 2008).

Given that the NLR protein family is involved in many biological processes and functions as proinflammatory receptors as well as negative regulators, in certain aspects of the invention, some NLR members may play a critical regulatory role in the control of NF-κB and type I interferon signaling. The present invention concerns the identification of NLRC5 as a potent negative regulator of NF-κB and IRF3 activation. NLRC5 strongly inhibits NF-κB-dependent responses by interacting with IKKα and IKKβ and blocking their phosphorylation. It also interacts with RIG-I and MDA5, but not with MAVS, to potently inhibit RLR-mediated type I interferon responses. Consistent with these observations, NLRC5-specific siRNA knockdown not only enhanced the activation of NF-κB and its responsive genes, TNF-α and IL-6, but also promoted type I interferon signaling and antiviral immunity. As a key negative regulator of NF-κB and type I interferon signaling, NLRC5 is a useful target for manipulating immune responses against infectious or inflammation-associated diseases, including cancer, in particular embodiments of the invention.

In certain aspects of the invention, the expression of NLRC5 is knocked down to treat a medical condition, such as for use as a cancer vaccine or as part of a cancer vaccine, or for an infectious disease. In specific aspects, an agent that modulates expression of NLRC5 is comprised in a dendritic cell.

In other aspects of the invention, NLCR5 is overexpressed in the tumor cells of an individual as treatment for cancer. The overexpression of NLCR5 may occur through delivery of part of all of NLRC5 in a vector or in a liposome, for example.

### BRIEF SUMMARY OF THE INVENTION

The present invention is directed to a system and method that concerns enhancement of an immune response in an individual. In particular, the invention relates to the enhanced immunity in the individual. In certain embodiments, the invention concerns targeting NLRC5 mRNA and/or NLRC5 protein to enhance immune response against microbial infections and inflammation-associated diseases. In specific aspects of the invention, reduction in expression of NLRC5 results in enhanced immune response against at least one microbial infection or inflammation-associated disease. In particular cases, an agent that reduces expression of NLRC5 or protein production is delivered to an individual in need thereof, which may be considered to be a person that has a need for or is at risk for needing enhanced immunity against a microbial infection or inflammation-associated disease. The individuals may be of any age. In specific embodiments, the microbial infection is a viral infection, a bacterial infection or a fungal infection.

In certain embodiments, the present invention provides siRNAs, pharmaceutical compositions comprising the siRNAs, in vitro and in vivo methods of inhibiting expression of NLRC5, and methods of enhancing an immune response. Accordingly, in certain cases the invention provides an isolated siRNA comprising a sense RNA strand and an antisense RNA strand, or a single RNA strand, wherein the sense and the antisense RNA strands, or the single RNA strand, form an RNA duplex, and wherein the RNA strand comprises a nucleotide sequence identical to a target sequence, for example a target sequence of about 15 to about 30 contiguous nucleotides in NLRC5 mRNA or mutant or variant thereof. “Short interfering RNA” (siRNA), also referred to herein as “small interfering RNA” or “siRNA” is defined as an agent that functions to inhibit expression of a target gene, e.g., by RNAi. An siRNA may be chemically synthesized, it may be produced by in vitro transcription, or it may be produced within a host cell, in particular embodiments.

In one embodiment, siRNA is a double stranded RNA (dsRNA) molecule of about 15, 16, 17, 18, 19, 20, 21, 22, 23, 24, 25, 26, 27, 28, or 30 nucleotides in length. In one embodiment the length is about 15 to about 30 nucleotides. In another embodiment, the length is about 19, 20, 21, 22, 23, 24, or 25 nucleotides in length. In yet another embodiment the length is about 19, 20, 21, 22, or 23 nucleotides in length and may contain a 3′ and/or 5′ overhang on each strand having a length of about 1, 2, 3, 4, or 5 nucleotides. The length of the overhang is independent between the two strands, i.e., the length of the over hang on one strand is not dependent on the length of the overhang on the second strand. In one embodiment, the siRNA is capable of promoting RNA interference through degradation or specific post-transcriptional gene silencing (PTGS) of the target messenger RNA (mRNA).

The invention also provides a recombinant vector comprising nucleic acid sequences for expressing an siRNA comprising a sense RNA strand and an antisense RNA strand, or a single strand, wherein the sense and the antisense RNA strands, or the single strand, form an RNA duplex, and wherein the RNA strand comprises a nucleotide sequence identical to a target sequence of about 15 to about 30 contiguous nucleotides in NLRC5 mRNA or mutant or variant thereof.

The invention also provides a pharmaceutical composition comprising at least one siRNA and a pharmaceutically acceptable carrier, wherein the siRNA comprises a sense RNA strand and an antisense RNA strand or a single strand, wherein the sense and the antisense RNA strands or the single strand form an RNA duplex, and wherein the RNA strand comprises a nucleotide sequence identical to a target sequence of about 15 to about 30 contiguous nucleotides in NLRC5 mRNA, or an alternative splice form, mutant or cognate thereof.

Negative regulators of innate immune signaling are critical in the maintenance of immune homeostasis and tumorigenesis (Komuro et al., 2008; Geddes et al., 2009). CYLD and A20 are deubiquitinases that negatively regulate the NF-κB signaling pathway. Mutations in CYLD and A20 have recently been identified in familial cylindromatosis and lymphoma (Trompouki et al., 2003; Compagno et al., 2009; Kato et al., 2009; Schmitz et al., 2009). These studies further support the embodiments wherein mutated NLRC5 plays a role in cancer development. Overexpression of NLRC5 in cancer cells blocks NF-kB and type I interferon pathways and thus inhibit tumor growth, in particular aspects of the invention.

It has become clear that negative regulators play a critical role in inhibiting the innate immune response and thus adaptive immunity. Elimination or blocking of NLRC5 in dendritic cells increases their capacity to induce robust immune responses against cancer and infectious diseases, in particular embodiments of the invention. However, because Treg cells can potently inhibit both CD4+ and CD8+T cell responses, overcoming Treg cell-mediated immune suppression is a prerequisite for generating potent and durable immunity. Thus, in certain embodiments, NLRC5-silencing dendritic cell vaccines combined with blocking Treg cell function treatment achieves optimal antitumor immunity. In particular aspects, the methods and compositions of the present invention are administered with agents that block Treg cell function, such as a non CpG containing oligonucleotide, including one between about 4 and about 15 nucleotides, for example one comprising a guanine and a nuclease-resistant inter-residue backbone linkage, for example one comprising a nuclease-sensitive inter-residue backbone linkage, or for example a guanine and a nuclease-resistant inter-residue backbone linkage connecting the guanine an adjacent nucleobase.

In one embodiment of the invention, there is a method of enhancing an immune response against microbial infection or inflammation-associated disease in an individual, comprising the step of providing to the individual an agent that inhibits NLRC5 in cells of the individual. In specific embodiments, the agent inhibits NLRC5 mRNA or protein. In specific cases, the agent is nucleic acid, polypeptide, or a small molecule. In particular embodiments, the NLRC5 siRNA, such as, for example, NLRC5 siRNA targets at least part of the region of the NLRC5 mRNA that encodes the CARD-like domain, the central NOD domain, the LRR region, the region that interacts with NFκB, the region that interacts with IKKα, or the region that interacts with IKKIβ. In a particular case, the NLRC5 siRNA targets at least part of the region of amino acids 900-1329 of NLRC5 polypeptide.

In some embodiments, the microbial infection is a bacterial infection, such as tuberculosis, pneumonia, foodborne illnesses, tetanus, typhoid fever, diphtheria, syphilis or leprosy; in some embodiments, the microbial infection is a viral infection, such as HIV, smallpox, influenza, mumps, measles, chickenpox, ebola, meningitis, or rubella, and in some embodiments, the microbial infection is a fungal infection, such as a yeast infection, jock itch, or athlete's foot.

In particular embodiments, an agent of the invention is provided to the individual by intradermal, transdermal, transmucosal, parenteral, intracranial, intravenous, intramuscular, intranasal, intracerebrospinal, subcutaneous, percutaneous, intratracheal, intraperitoneal, intratumoral, perfusion, lavage, direct injection, and/or oral administration. The agent may be delivered to the individual in a dendritic cell or in a liposome.

In specific embodiments of the invention, methods of the invention further include the step of delivering to the individual an agent that blocks Treg cell function, such as one that is a non CpG containing oligonucleotide. In specific embodiments, the oligonucleotide has one or more of the following characteristics: it is between about 4 and about 15 nucleotides; it comprises a guanine and a nuclease-resistant inter-residue backbone linkage; it comprises a nuclease-sensitive inter-residue backbone linkage; and it comprises a guanine and a nuclease-resistant inter-residue backbone linkage connecting the guanine and an adjacent nucleobase.

In an embodiment of the invention, there is a method of treating an individual for cancer or preventing cancer in an individual, comprising the step of delivering to the individual an agent that increases NLRC5 in cancer cells of the individual. In a specific embodiment, the agent is NLRC5 polypeptide. In a particular case, the agent is an expression construct that is capable of expressing NLRC5. In a specific embodiment, the agent that increases NLRC5 is comprised within a liposome.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1 shows domain organization, expression of human and mouse NLRC5 and its intracellular localization. (A) Domain organization of human and mouse NLRC5 proteins. CARD: caspase recruitment domain, NOD: Nucleotide binding domain, LRR: Leucine rich repeats. (B) Expression of HA-tagged NLRC5 and mNLRC5 proteins in 293T cells. (C) mRNA expression profiles of human and mouse NLRC5 by Real-time PCR analysis. (D) Immunoblot analysis of human and mouse NLRC5 expression in the indicated cell lines or primary cells. Exogenous expressed HA-NLRC5 and HA-NLRC5 in 293T cells were taken as the positive control. (E) Intracellular localization of NLRC5. 293T cells were transfected with either pcDNA3.1-GFP-NLRC5 or pcDNA3.1-GFP and analyzed by fluorescence microscope. The nuclei were stained by DAPI. (F) and (G) show the entire image of FIG. 1D.

FIG. 2 shows cloning of human and mouse NLRC5 genes. (A) The N-terminal and C-terminal fragments of human NLRC5 were obtained by PCR amplification. The 3.5-kb N-terminal fragment and the C-terminal 2.1-kb fragment were then cloned into a pcDNA-HA vectors. (B) The N-terminal and C-terminal fragments of mouse NLRC5 were amplified, as described for human NLRC5. Both fragments were subsequently cloned into the pcDNA-HA to get the final plasmid pcDNA-HA-mNLRC5.

FIG. 3 shows analysis of human and mouse NLRC5 proteins. (A) The predicted amino acid sequences of mouse NLRC5 protein (having one amino acid difference from GenBank® NM_{—}001033207 at position 881 of the protein, wherein the inventive sequence has an arginine instead of a glycine; NM_{—}001033207 is SEQ ID NO:12, and SEQ ID NO:13 is the corresponding mRNA) is shown in FIG. 3A (human NLRC5 protein is provided in SEQ ID NO:5). (B) Sequence alignment of various NLRC5 homologues in mammals and birds. The protein sequence of mouse NLRC5 is predicted on the basis of full-length cDNA generated in the invention. Other sequences are obtained from the database of NCBI. The protein accession numbers of dog, bull, horse, human and chicken NLRC5 are XM_{—}544394 (SEQ ID NO:2), XP_{—}001250847 (SEQ ID NO:3), XP_{—}001915536 (SEQ ID NO:4), NP_{—}115582 (SEQ ID NO:5) and XP_{—}001232361 (SEQ ID NO:6), respectively. Sequences of exemplary corresponding NLRC5 mRNAs are in SEQ ID NO:7 (dog), SEQ ID NO:8 (bull), SEQ ID NO:9 (horse), SEQ ID NO:10 (human), and SEQ ID NO:11 (chicken).

FIG. 4 demonstrates the specificity of anti-NLRC5 and anti-mNLRC5 antibodies. (A) Specificity of NLRC5 antibody for exogenous and endogenous protein was determined by immunoblot analysis using HEK293T cells expressing NLRC5. (B) Specificity of mNLRC5 antiserum. HA-mNLRC5 was expressed in HEK293T cells and pulled down with anti-HA beads, and then analyzed with anti-HA and anti-mNLRC5 antibodies.

FIG. 5 shows that NLRC5 inhibits NF-κB activation induced by IL-1β, LPS, TNF-α and their downstream signalling molecules. (A) Effects of NLRC5 on NF-κB-luciferase activation (fold induction). 293T cells were transfected with an NF-κB-luc reporter plasmid and TLR4 plasmid (for LPS treatment), together with an empty vector or NLRC5 construct, and analyzed for NF-κB-dependent luciferase activity after treatment with IL-1β (20 ng/ml), LPS (1 μg/ml) or TNF-α (50 ng/ml). (B) NLRC5 inhibits NF-κB-luciferase activation by MyD88, TRAF6, IKKα, or IKKβ, but not by p65. (C) NF-κB activation is not inhibited by the NLR proteins NOD1 and NOD2. (D) Detection of endogenous NF-κB activity in the formation of DNA complexes at NF-κB binding sites in a gel-mobility shift assay. Oct-1/DNA binding complexes served as a loading control for nuclear extracts. (E) Human THP-1 cells and murine embryonic fibroblasts (MEF) were transfected with the NF-κB-luc reporter plasmid, together with (or without) NLRC5 or mNLRC5 plasmid, and then analyzed for NF-κB-dependent luciferase activity after LPS treatment.

FIG. 6 demonstrates that mouse NLRC5 functions as a negative regulator as human NLRC5 in the inhibition of NF-κB activation. mNLRC5 could inhibit MyD88, TRAF6, IKKα, IKKβ but not p65, induced NF-κB activation. The data are presented as means±s.d. in three independent experiments.

FIG. 7 shows that NLRC5 interacts with IKKα and IKKIβ to inhibit their phosphorylation. (A) Communoprecipitation of HA-tagged NLRC5 with Flag-tagged IKKα, IKKβ or NEMO. 293T cells were transfected with Flag-IKKα, IKKβ, Flag-NEMO and HA-NLRC5. HA-tagged NLRC5 protein was immunoprecipitated with anti-HA beads, and blotted with anti-Flag. Whole cell extracts were analyzed by Western blot analysis for expression of various proteins. (B) Interaction between endogenous NLRC5 and IKKα/β. 293T and RAW264.7 cell extracts were immunoprecipitated with IgG, anti-NLRC5 or anti-mNLRC5 antibody respectively, and then analyzed together with whole cell extracts by Western blot with an anti-IKKα/β, anti-NEMO, anti-NLRC5 or anti-mNLRC5 antibody. (C) Competitive binding between NLRC5 and NEMO to IKKIβ. 293T cells were transfected with indicated doses of Flag-NLRC5, HA-IKKβ and HA-NEMO. Whole cell extracts were immunoprecipitated with anti-Flag beads, and blotted with anti-HA. (D) Deletion mutants of IKKβ used in domain-mapping experiments. Numbers in parentheses indicate amino acids included in construct. LZ, leucine zipper; HLH, helix-loop-helix. (E) NLRC5 binds to the kinase domain of IKKIβ. 293T cells were transfected with HA-NLRC5 and Flag-IKKβ or various Flag-IKKβ mutants. Whole cell extracts were immunoprecipitated with anti-Flag beads, and blotted with anti-HA. (F) NLRC5 inhibits the phosphorylation of IKKα and IKKβ. (G) NLRC5 has no effects on the phosphorylation of p38 and JNK. In (F) and (G), 293T cells were transfected with IKKα, JNK1, JNK2, and p38 with or without HA-NLRC5. Whole cell extracts were used for Western blot analysis.

FIG. 8 shows that endogenous NLRC5 interacts with IKKα and IKKβ. HEK293T and RAW264.7 cell extracts were immunoprecipitated with anti-IKKα/β anti-NEMO or control IgG, respectively, and then analyzed by Western blot with an anti-NLRC5 or mNLRC5 antibody. Whole cell extracts were analyzed for IKKα/β, NEMO, NLRC5 or mNLRC5 expression by Western blot.

FIG. 9 shows interaction and functional analysis of NLRC5 deletions in the inhibition of NF-κB activation. (A) Generation and expression of NLRC5 deletion constructs. (B) and (C) The interaction between NLRC5 mutations and IKKβ or kinase domain of IKKβ. 293T cells were transfected with NLRC5 and its mutations constructs, IKKIβ or kinase domain of IKKβ and analyzed by coimmunoprecipitation and Western blot. (D) Functional activity of each NLRC5 deletion mutant. 293T cells were transfected with the NF-κB-luc reporter, together with an empty vector, or with full-length NLRC5 and its deletion constructs, and analyzed for luciferase activity (fold induction). (E) The NLRC5-D3 construct strongly inhibits the phosphorylation of IKKα and IKKβ, while other deletion constructs show only weak inhibition.

FIG. 10 shows knockdown of NLRC5 can significantly enhance NF-κB activation and inflammatory responses. (A) Immunoblot analysis of NLRC5 or mNLRC5 in 293T cells, THP-1 cells and RAW264.7 cells treated with NLRC5/mNLRC5 siRNA or scrambled siRNA. (B) Knockdown of endogenous mNLRC5 markedly enhanced the phosphorylation of IKK and IKB, but has no effects on the phosphorylation of JNK, ERK and p38. RAW264.7 cells were transfected with mNLRC5 siRNA or scrambled siRNA, and then treated with LPS (100 ng/ml). The cell extracts were harvested at different time points and used for Western blot of various kinases and signalling proteins. (C) Specific knockdown of NLRC5 or mNLRC5 enhanced NF-κB-luc activity in 293T/TLR4 and RAW264.7 cells after LPS treatment (100 ng/ml) compared with cells transfected with scrambled siRNA. (D) Knockdown of endogenous mNLRC5 markedly enhanced the DNA binding activity of NF-κB. RAW264.7 cells were transfected with NLRC5 siRNA or scrambled siRNA, and treated with LPS for 1 h; the nuclear proteins were harvested for NF-κB binding activity determined by EMSA. OCT-1 DNA-binding complexes were served as a control. (E) siRNA-mediated knockdown of endogenous NLRC5 enhanced TNF-α and IL-6 production in THP-1 and RAW264.7 cells after LPS treatment. Data in panels (C) and (E) are presented as means±s.d. Asterisks indicate significant differences in cytokine production after LPS treatment between cells transfected with NLRC5 siRNA or scrambled siRNA (** P<0.01, *** P<0.001 as determined by Student's t-test).

FIG. 11 demonstrates specific knockdown of NLRC5 and mNLRC5 by the corresponding NLRC5 siRNA and mNLRC5 siRNA in different cell lines. (A) Specific knockdown of exogenous NLRC5 expression by NLRC5 siRNA. NLRC5 Stealth™ Select RNAi oligonucleotide (Invitrogen) and scramble siRNA as well as HA-NLRC5 plasmid were transfected into 293T cells for 48 h. Knockdown efficiency was analyzed by Western blotting with anti-HA antibody. (B) Transfection efficiency of THP-1 cells with pmaxGFP (Lonza) determined by FACS analysis. (C) Specific knockdown of endogenous NLRC5 expression by NLRC5 and mNLRC5 siRNAs in 293T, THP-1 and RAW264.7 cells. For 293T cells, Lipofectamine 2000 (Invitrogen) was used for transfection. For THP-1 and RAW264.7 cells, Nucleofector Kit V (Lonza) was used for transfection.

FIG. 12 shows knockdown of NLRC5 by siRNA enhances the activation of the NF-κB pathway signaling proteins in THP-1 cells. THP-1 cells were transfected with NLRC5-specific siRNA or scramble siRNA, and then treated with 200 ng/ml LPS. The phosphorylation of IKK was enhanced and the degradation of IκB accelerated in cells transfected with NLRC5 siRNA, compared the cells with scramble siRNA. However, the phosphorylation of JNK, ERK and p38 was essentially unchanged between cells transfected with NLRC5 and scramble siRNAs.

FIG. 13 shows that NLRC5 negatively regulates IFN-β, activation by inhibiting RIG-I and MDA5 function. (A) and (B): 293T cells were transfected with INF-β-luc or ISRE-luc, NLRC5 plus poly(I:C)/Lyovec, RIG-I, MDA5, MAVS, TBK1 or IKKi plasmids and analyzed for INF-13 or ISRE luciferase activity. Values are means±s.d of three independent experiments. (C) 293T cells were transfected with HA-NLRC5 plus RIG-I, MAVS or IKKi. After immunoprecipitation with anti-HA beads, specific proteins were analyzed by Western blot with anti-Flag. (D) Interaction of mNLRC5 and RIG-I after VSV-eGFP infection. RAW264.7 cells were infected with VSV-eGFP, and cell extracts were harvested at different time points, immunoprecipitated with anti-mNLRC5 antibody and analyzed by Western blot with anti-RIG-I. (E) 293T cells were transfected with MDA5 with or without HA-NLRC5. After immunoprecipitation with anti-HA beads, specific proteins were analyzed by Western blot with anti-MDA5. (F) 293T cells were transfected with Flag-RIG-I and Flag-MAVS, with or without HA-NLRC5. Cell extracts were blotted by anti-phospho-IRF3 and IRF3 antibodies. (G) RAW265.7 and THP-1 cells were transfected with NLRC5/mNLRC5-specific siRNA or scrambled siRNA, respectively, and then infected with VSV-eGFP. The cell extracts were harvested for Western blot with anti-phospho-IRF3 and IRF3 antibodies.

FIG. 14 shows functional conservation between human NLRC5 and mNLRC5 proteins in inhibiting IFN-β activation. mNLRC5 showed strong inhibitory effects on RIG-I and MDA5 induced IFN-β activation, and weak effects on MAVS induced IFN-β activation. The data are presented as means±s.d. in three independent experiments.

FIG. 15 demonstrates that there is no interaction between NLRC5 with TRIF, TRAF3, TBK-1 or IRF3. Cell lysates were immunoprecipitated by anti-Flag or anti-HA beads, and then analyzed by Western blotting with anti-Flag or anti-HA antibody. Protein expression of NLRC5, TRIF, TRAF3, TBK-1 or IRF3 in whole cell lysates was confirmed by immunolot with anti-Flag or anti-HA antibodies.

FIG. 16 shows that knockdown of NLRC5 enhances antiviral immunity. (A) and (B) RAW264.7 cells or THP-1 cells were transfected with mNLRC5/NLRC5-specific siRNA or scrambled siRNA, followed by poly(I:C)/Lyovec treatment. ISG-54, ISG-56, IFN-β mRNA and IFN-β protein were evaluated. (C) RAW264.7 cells, murine macrophages and human monocytes were transfected with mNLRC5/NLRC5-specific siRNA or scrambled siRNA, followed by VSV-eGFP infection. IFN-β protein was evaluated. The data are presented as means±s.d of three independent experiments. Asterisks indicate significant differences in mRNA or protein levels after poly(I:C)/Lyovec treatment or viral infection between cells transfected with NLRC5 siRNA and scrambled siRNA (* P<0.05, ** P<0.01, *** P<0.001 determined by Student's t-test). (D) 293T cells, THP-1 cells and RAW264.7 cells were transfected with NLRC5/mNLRC5-specific siRNA or scrambled siRNA, and then infected with VSV-eGFP. Viral infections were analyzed by fluorescence microscopy (with phase contrast as a control) as well as FACS analysis. (E) Proposed model of NLRC5 as a negative regulator that blocks both IKK complexes and RIG-I/MDA5 activation and function.

FIG. 17 demonstrates that knockdown of NLRC5 in THP-1 cells enhances IFN-β response. Knockdown of NLRC5 in THP-1 can enhance IFN-β production by VSV-eGFP infection at both the mRNA and protein levels.

FIG. 18 demonstrates that knockdown of NLRC5 in different type of cells enhances the antiviral immunity. (A) HEK293T cells, THP-1 cells and RAW264.7 cells were transfected with NLRC5-specific siRNA or scramble siRNA, and then infected with VSV-eGFP. Viral infections were analyzed by fluorescence microscopy. (B) MEF and human monocytes were transfected with NLRC5-specific siRNA or scramble siRNA, and then infected with VSV-eGFP. Viral infections were analyzed by fluorescence microscopy (with phase contrast as a control).

FIG. 19 shows that human and mouse NLRC5 are up-regulated by NF-κB. (A) The mRNA expression level of NLRC5 is upregulated by LPS and TNF-α stimulation in 293T/TLR4 cells. (B) mRNA expression change of NLRC5 and NLRX1 by LPS treatment in THP-1 cells and RAW264.7 cells. NLRC5 is up-regulated by LPS and TNF-α while NLRX1 is not. The data are presented as means±s.d. in three independent experiments. (C) NLRC5 protein expression change after LPS treatment in RAW264.7 cells.

FIG. 20 shows that NLRC5 can be disassociated from IKKs after LPS stimulation. RAW264.7 cells were treated with 200 ng/ml LPS and total cell lysate was collected in the indicated time points to immunoprecipitate with anti-mNLRC5 followed by blotting with anti-IKKα or anti-IKKβ antibody.

FIG. 21 demonstrates that NLRC5/IKKα/β complex and IKKα/β/NEMO complex co-exist in resting cells. (A) Cell extracts of HEK293T cells expressing Flag-NLRC5 were immunoprecipitated with anti-IKKα, anti-Flag, anti-NEMO or control IgG antibody, and then immunoblotted with the indicated antibodies. (B). Cell extracts of RAW264.7 cells were fractionated on a size-exclusion column (HiPrep 16/60 Sephacryl S-300 HR). Factions collected were resolved on SDS-polyacrylamide gel electrophoresis and immunoblotted with anti-mNLRC5, anti-IKKα/β or anti-NEMO antibody.

FIG. 22 shows that RAW264.7 cells were transfected with mNLRC5 siRNA or scrambled siRNA for 36 h and then treated with LPS (200 ng/ml), poly (I:C)/LyoVec (1 μg/ml) or VSV-eGFP infection. Total RNA from the treated cells was harvested at different time points and used for realtime PCR analysis for detecting the gene expression of TNF-α, IL-6, IFN-α and IFN-β. (* P<0.05, ** P<0.01, *** P<0.001 determined by Student's t-test).

FIG. 23 demonstrates dynamic interaction between NLRC5 and IKKα/IKKβ over time after LPS stimulation. (A) RAW264.7 cells were treated with 200 ng/ml LPS and total cell lysates were collected at the indicated time for immunoprecipitation with anti-mNLRC5, followed by immunoblotting with anti-IKKα, anti-IKKβ and anti-NEMO antibody. Whole cell lysates were immunobolted with the indicated antibodies. (B) The interaction between NLRC5 and IKKα/IKKβ over period 2-18 h after treatment. (C) Analysis of band density in 23A and 23B for p-IKK and NLRC/IKK pulled down by anti-NLRC5 (i.e. NLRC5/IKK complex).

FIG. 24 shows that RAW264.7 cells were treated with LPS (200 ng/ml), poly (I:C)/LyoVec and VSV-eGFP. Cell extracts and RNA were collected for further analysis. (A) Realtime PCR was performed to determine the mRNA expression level of mNLRC5 at indicated time points. (B) mNLRC5 and beta-actin were immunoblotted by anti-mNLRC5 and anti-β-actin after treatment. The density of bands was scanned and analyzed by B and Scan software. The band density of β-actin was used to normalize the data. (C). MyD88−/− or wild type mouse peritoneal macrophages were treated with LPS (200 ng/ml) and RNA were collected for real-time PCR analysis.

FIG. 25 shows that endogenous mNLRC5 interacts with IKKα/β. RAW264.7 cell extracts were immunoprecipitated with anti-IKKα/β, anti-NEMO or control IgG, respectively, and then analyzed by Western blot with an anti-NLRC5 or mNLRC5 antibody. Whole cell extracts were analyzed for IKKα/β, NEMO, or mNLRC5 expression by Western blot.

FIG. 26. (A). Inhibition of NF-κB-luc, IFN-β-luc and ISRE-luc activity by LPS treatment in 293T/TLR4 in the presence of increasing doses (0, 100 and 200 ng) of NLRC5 or NLRC5 siRNAs. (B) NLRC5 can not inhibit ISRE-luc activity induced by overexpression of TRIF in 293T cells. (C). Inhibition of NF-κB-luc, IFN-β-luc and ISRE-luc activity by R848 treatment in 293T/TLR7 of NLRC5 (0 or 200 ng). (* P<0.05, ** P<0.01, *** P<0.001 determined by Student's t-test).

FIG. 27 shows that NLRC5 inhibits NF-κB-luc and IFN-β-luc activity induced by poly (I:C)/LyoVec treatment or overexpression of MDA5, RIG-I or MAVS in 293T cells. (* P<0.05, ** P<0.01, *** P<0.001 determined by Student's t-test).

FIG. 28 shows that RAW264.7 cells were transfected with scrambled siRNA or mNLRC5 siRNA for 36 h and then infected with VSV-eGFP. The images of fluorescence and phase-contrast were taken at 0, 6, 8, 10, 12, 14, 16, 18 h (A), 15 h (B), 20 h (C) to monitor the propagations of VSV-eGFP.

FIG. 29 demonstrates that 293T/TLR4 cells were transfected with NLRC5-GFP for 24 h and treated with 1 μg/ml LPS. Mito-tracker was used to stain mitochondria (A) and GFP-NLRC5 distribution was monitored within 1 h (B). (C) 293T/TLR4 cells were treated with 1 μg/ml LPS. NLRC5 polyclone antibody was used to detect endogenous NLRC5. Pre-immune serum was used as a negative control.

FIG. 30 shows that RAW264.7 cells were transfected with scrambled siRNA or mNLRC5 siRNA for 36 h and treated with LPS (200 ng/ml) or VSV-eGFP. Cell supernatants were collected at 10 h to measure cytokine production with ELISA kits. (* P<0.05, ** P<0.01, *** P<0.001 determined by Student's t-test).

FIG. 31 demonstrates competitive binding between NLRC5-D3 or NLRC5-D2 and NEMO to IKKβ. 293T cells were transfected with indicated doses of Flag-NLRC5-D3 or Flag-NLRC5-D2, HA-IKKβ and HA-NEMO. Whole cell extracts were immunoprecipitated with anti-Flag beads, and blotted with anti-HA.

FIG. 32 shows analysis of band density in FIG. 10B. (A) p-IKK, (B) IκB, (C) p-JNK. All the data were normalized by β-actin and p38.

FIG. 33 (A) RAW264.7 were infected with VSV-eGFP and cell extracts were collected at different time points, immunoprecipitated with anti-mNLRC5 antibody and analyzed by Western blot with the indicated antibodies. (B) Analysis of band density of RIG-I in FIG. 13D and FIG. 33A. All the data were normalized by β-actin. (C) 293T cells were transfected with HA-NLRC5 plus Flag-RIG-I, Flag-RIG-I CARD domain and Helicase domain (HD). After immunoprecipitation with anti-Flag beads, specific proteins were analyzed by Western blot with anti-HA. (D) HEK293T cells were transfected with Flag-MAVS-HA plus Flag-RIG-1, or Flag-NLRC5. After immunoprecipitation with anti-HA beads, specific proteins were analyzed by western blot with anti-Flag. (E) Proposed model of the negative regulation of RIG-I activation by NLRC5.

FIG. 34 (A). Anti-NLRC5 can recognize endogenous NLRC5 and HA-NLRC5. (B). Anti-NLRC5 can specifically recognize Flag-NLRC5 but not Flag-NLRX1. (C). Anti-NLRC5 can not recognize mNLRC5 in RAW264.7 cells. (D). anti-mNLRC5 can recognize HA-mNLRC5. (E). Anti-mNLRC5 can specifically recognize endogenous mNLRC5 in RAW264.7 cells.

FIG. 35 shows that 293T cells were transfected with 1 ng p65 and increasing doses of NLRC5 (0, 50, 100, 200 ng), NF-κB-luc and TK-Renilla-luc for 24 h, and then analyzed by luciferase assay.

FIG. 36 shows that RAW264.7 or THP-1 cells were transfected with the indicated siRNA for 36 h. Total RNA were collected for RT-PCR and real-time-PCR analysis (A). 36 h post transfection of siRNAs, RAW264.7 and THP-1 cells were treated with 200 ng/ml LPS for 6 h and medium was collected for ELISA analysis (B). (* P<0.05, ** P<0.01, *** P<0.001 determined by Student's t-test).

### DETAILED DESCRIPTION OF THE INVENTION

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

Applicants incorporate by reference herein in their entirety U.S. patent application Ser. No. 11/757,235, filed Jun. 1, 2007, and U.S. patent application Ser. No. 11/816,902, filed Mar. 9, 2006.

### I. DEFINITIONS

As used herein, the term “immune response” is defined as the mechanisms, including the synthesis of antibodies, that are employed in the body to respond to the presence of a foreign antigen or cancer.

As used herein, the term “immunity” is defined as the host being protected from challenge of invading pathogens or cancer. In particular embodiments, this includes innate immunity as well humoral and cellular adaptive immunity.

Stringent control of the NF-κB and type I interferon signaling pathways is critical to effective host immune responses. In the present invention, NLRC5, a member of the highly conserved NOD-like protein family, inhibits the IKK complex and RIG-I/MDA5 function. NLRC5 strongly inhibited NF-κB-dependent responses by interacting with IKKcL and IKKβ and blocking their phosphorylation. It also interacted with RIG-I and MDA5, but not with MAVS, to potently inhibit RLR-mediated type I interferon responses. Consistent with these observations, NLRC5-specific siRNA knockdown not only enhanced the activation of NF-κB and its responsive genes, TNF-α and IL-6, but also promoted type I interferon signaling and antiviral immunity. The invention identifies NLRC5 as a key negative regulator that blocks two central components of the NF-κB and type I interferon signaling pathways, and hence a pivotal element in the homeostatic control of the immune system.

In certain embodiments of the invention, RNA interference (in particular siRNA) is employed to target NLRC5 to enhance an immune response. RNA interference (also referred to as “RNA-mediated interference”; RNAi) is a mechanism by which gene expression can be reduced or eliminated. Double stranded RNA (dsRNA) or single stranded RNA has been observed to mediate the reduction, which is a multi-step process (for details of single stranded RNA methods and compositions see Martinez et al., 2002, for example). dsRNA activates post-transcriptional gene expression surveillance mechanisms that appear to function to defend cells from virus infection and transposon activity (Fire et al., 1998; Grishok et al., 2000; Ketting et al., 1999; Lin et al., 1999; Montgomery et al., 1998; Sharp et al., 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene. (Fire et al., 1998; Grishok et al., 2000; Ketting et al., 1999; Lin et al., 1999; Montgomery et al., 1998; Sharp, 1999; Sharp et al., 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp, 1999; Sharp et al., 2000; Elbashir et al., 2001).

SiRNAs are small RNAs that do not significantly induce the antiviral response common among vertebrate cells but that do induce target mRNA degradation via the RNAi pathway. The term siRNA refers to RNA molecules that have either at least one double stranded region or at least one single stranded region and possess the ability to effect RNAi. It is specifically contemplated that siRNA refers to RNA molecules that have at least one double stranded region and possess the ability to effect RNAi, in specific embodiments. In certain embodiments, mixtures or pools of dsRNAs (siRNAs) may be generated by various methods including chemical synthesis, enzymatic synthesis of multiple templates, digestion of long dsRNAs by a nuclease with RNAse III domains, and the like. A “pool” or “cocktail” refers to a composition that contains at least two siRNA molecules that have different selectivity with respect to each other, but are directed to the same target gene. Two or more siRNA molecules that have different selectivity with respect to each other, but are directed to the same or different target gene(s) are defined as different siRNAs. Different siRNAs may overlap in sequence and may contain two sequences that are contiguous or non-contiguous in the target gene. In some embodiments, a pool contains at least or at most 3, 4, 5, 6, 7, 8, 9, 10 or more siRNA molecules. An “siRNA directed to” a particular region or target gene means that a particular siRNA includes sequences that results in the reduction or elimination of expression of the target gene, i.e., the siRNA is targeted to the region or gene. The pool in some embodiments includes one or more control siRNA molecules. In other embodiments a control siRNA molecule is not included in the pool. A pool of siRNA molecules may also contain various candidate siRNA molecules that do not reduce or eliminate expression of a target gene.

### II. Short Interfering RNAs (siRNAs) of the Invention

In one embodiment, the siRNA are provided that are useful in the methods of treatment and prevention of microbial infection or inflammation-associated disease. Other siRNAs useful in prevention or treatment according to the methods of the present invention may be readily designed and tested. Accordingly, the present invention in some cases relates to siRNA molecules of about 15 to about 30 or about 15 to about 28 nucleotides in length, which are homologous to a NLRC5 gene. In one embodiment, the siRNA molecules have a length of about 19 to about 25 nucleotides. In another embodiment, the siRNA molecules have a length of about 19, 20, 21, or 22 nucleotides. The siRNA molecules of the present invention can also comprise a 3′ hydroxyl group. The siRNA molecules can be single-stranded or double stranded; such molecules can be blunt ended or comprise overhanging ends (e.g., 5′, 3′). In specific embodiments, the RNA molecule is double stranded and either blunt ended or comprises overhanging ends.

In one embodiment, at least one strand of the RNA molecule has a 3′ overhang from about 0 to about 6 nucleotides (e.g., pyrimidine nucleotides, purine nucleotides) in length. In other embodiments, the 3′ overhang is from about 1 to about 5 nucleotides, from about 1 to about 3 nucleotides and from about 2 to about 4 nucleotides in length. In one embodiment the RNA molecule is double stranded, one strand has a 3′ overhang and the other strand can be blunt-ended or have an overhang. In the embodiment in which the RNA molecule is double stranded and both strands comprise an overhang, the length of the overhangs may be the same or different for each strand. In a particular embodiment, the RNA of the present invention comprises about 19, 20, 21, or 22 nucleotides which are paired and which have overhangs of from about 1 to about 3, particularly about 2, nucleotides on both 3′ ends of the RNA. In one embodiment, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

In one embodiment, the siRNA of the present invention comprises two molecules where the sense RNA strand comprises one RNA molecule, and the antisense RNA strand comprises one RNA molecule, or the sense and antisense RNA strands forming the RNA duplex may be covalently linked by a single-stranded hairpin. In another embodiment, the siRNA is comprised of non-nucleotide material. In yet another embodiment, the sense and antisense RNA strands of the siRNA may be stabilized against nuclease degradation. The siRNA may contain one or two 3′ overhangs comprising from 1 to about 6 nucleotides each. Alternatively, the 3′ overhang is comprised of a dinucleotide of dithymidylic acid (TT) or diuridylic acid (uu). In yet another embodiment, the 3′ overhang is stabilized against nuclease degradation.

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety).

### III. Design and Preparation of siRNA Molecules

Synthetic siRNA molecules, including shRNA molecules, of the present invention can be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell. 9:1327-1333; Rubinson, D. A., et al. (2003) Nat. Genet. 33:401-406; Stewart, S. A., et al. (2003) RNA 9:493-501). These vectors generally have a polIII promoter upstream of the dsRNA and can express sense and antisense RNA strands separately and/or as a hairpin structures. Within cells, Dicer processes the short hairpin RNA (shRNA) into effective siRNA.

The targeted region of the siRNA molecule of the present invention can be selected from a given target gene sequence; in specific embodiments, the target sequence begins from about 25 to 50 nucleotides, from about 50 to 75 nucleotides, or from about 75 to 100 nucleotides downstream of the start codon. Nucleotide sequences may contain 5′ or 3′ UTRs and regions nearby the start codon. One method of designing a siRNA molecule of the present invention involves identifying the 23 nucleotide sequence motif AA(N19)TT (SEQ ID NO:1) (where N can be any nucleotide) and selecting hits with at least 25%, 30%, 35%, 40%, 45%, 50%, 55%, 60%, 65%, 70% or 75% G/C content. The “TT” portion of the sequence is optional, in certain cases. Alternatively, if no such sequence is found, the search may be extended using the motif NA(N21), where N can be any nucleotide. In this situation, the 3′ end of the sense siRNA may be converted to TT to allow for the generation of a symmetric duplex with respect to the sequence composition of the sense and antisense 3′ overhangs. The antisense siRNA molecule may then be synthesized as the complement to nucleotide positions 1 to 21 of the 23 nucleotide sequence motif. The use of symmetric 3′ TT overhangs may be advantageous to ensure that the small interfering ribonucleoprotein particles (siRNPs) are formed with approximately equal ratios of sense and antisense target RNA-cleaving siRNPs (Elbashir et al. (2001) supra and Elbashir et al. 2001 supra). Analysis of sequence databases, including but not limited to the NCBI, BLAST, Derwent and GenSeq as well as commercially available oligosynthesis companies such as Oligoengine®, may also be used to select siRNA sequences against EST libraries to ensure that only one gene is targeted.

The siRNAs as described herein including the NLRC5 targeting siRNA can be administered to individuals to treat microbial disease or inflammation-associated disease. In conjunction with such treatment, pharmacogenomics (i.e., the study of the relationship between an individual's genotype and that individual's response to a foreign compound or drug) may be considered. Differences in metabolism of therapeutics, including siRNAs, can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer one or more therapeutic siRNAs as described herein as well as tailoring the dosage and/or therapeutic regimen of treatment with an siRNA targeting a NLRC5 gene.

For example, in one embodiment, before administering the siRNA to an individual, the target sequence of the NLRC5 harbored by the individual may be analyzed for any potential gene variations, such as polymorphisms or mutations, in the region against which the siRNA is targeted. For example, one may sequence the NLRC5 gene from a sample from the individual. If one or more mutations or a polymorphisms is detected, the siRNA may be modified to target the specific mutant or polymorphic form of the target.

### IV. Delivery of siRNA Agents

Methods of delivering siRNA of the present invention, or vectors containing siRNA of the present invention, to the target cells, such as dendritic cells, for example, or all other body cells, include administration of a composition containing the siRNA, or directly contacting the target cell, with a composition comprising an siRNA. In another embodiment, an siRNA may be injected directly into any blood vessel, such as vein, artery, venule or arteriole, via methods including but not limited to hydrodynamic injection or catheterization. In certain cases, administration may be oral, intravenous, or by subcutaneous or intramuscular injection (by a single injection or by two or more injections, for example). The siRNA is delivered in a pharmaceutically acceptable carrier, in particular embodiments. One or more siRNAs targeting NLCR5 may be used simultaneously.

In one embodiment, only one siRNA that targets NLRC5 is used. The delivery or administration of the siRNA is in one embodiment performed in free form, i.e. without the use of vectors. In another embodiment, a mixture of siRNAs targeting either the same viral gene or at least 2, 3, 4, 5 or up to at least 10 different NLRC5 genes or gene variants are used.

In one embodiment, the compositions of the invention are provided as a surface component of a lipid aggregate, such as a liposome, or are encapsulated by a liposome. Liposomes, which can be unilamellar or multilamellar, can introduce encapsulated material into a cell by different mechanisms. For example, the liposome can directly introduce its encapsulated material into the cell cytoplasm by fusing with the cell membrane. Alternatively, the liposome can be compartmentalized into an acidic vacuole (i.e., an endosome) and its contents released from the liposome and out of the acidic vacuole into the cellular cytoplasm. In one embodiment the invention features a lipid aggregate formulation of the compounds described herein, including phosphatidylcholine (of varying chain length; e.g., egg yolk phosphatidylcholine), cholesterol, a cationic lipid, and 1,2-distearoyl-sn-glycero3-phosphoethanolamine-polyethyleneglycol-2000 (DSPE-PEG2000). The cationic lipid component of this lipid aggregate can be any cat ionic lipid known in the art such as dioleoyl 1,2-diacyl trimethylammonium-propane (DOTAP). In another embodiment, polyethylene glycol (PEG) is covalently attached to the compositions of the present invention. The attached PEG can be any molecular weight but is typically between 2000-50,000 daltons. In one embodiment for targeting macrophages for delivery of siRNA, liposomes containing of phosphatidyl serine may be used since macrophage engulfment via the phosphatidyl serine receptor promotes an anti-inflammatory response by increasing TGF-beta1 secretion (Huynh, M. L. et al. (2002) J. Cell Biol. 155, 649). Therefore, when the macrophages are successfully transfected, not only will the target genes be silenced, but the macrophage will also be induced to secrete anti-inflammatory cytokines.

In another embodiment, a polyG tail, e.g., a 5-10 nucleotide tail, may be added to the 5′ end of the sense strand of the siRNA, which in certain cases may enhance uptake via a cell receptor.

In another embodiment of the invention, the siRNA of the invention may be transported or conducted across biological membranes using carrier polymers which comprise, for example, contiguous, basic subunits, at a rate higher than the rate of transport of siRNA molecules which are not associated with carrier polymers. Combining a carrier polymer with siRNA, with or without a cationic transfection agent, results in the association of the carrier polymer and the siRNA. The carrier polymer may efficiently deliver the siRNA, across biological membranes both in vitro and in vivo. Accordingly, the invention provides methods for delivery of an siRNA, across a biological membrane, e.g., a cellular membrane including, for example, a nuclear membrane, using a carrier polymer. The invention also provides compositions comprising an siRNA in association with a carrier polymer.

The term “association” or “interaction” as used herein in reference to the association or interaction of an siRNA and a carrier polymer, refers to any association or interaction between an siRNA with a carrier polymer, e.g., a peptide carrier, either by a direct linkage or an indirect linkage. An indirect linkage includes an association between an siRNA and a carrier polymer wherein said siRNA and said carrier polymer are attached via a linker moiety, e.g., they are not directly linked. Linker moieties include, but are not limited to, e.g., nucleic acid linker molecules, e.g., biodegradable nucleic acid linker molecules. A nucleic acid linker molecule may be, for example, a dimer, trimer, tetramer, or longer nucleic acid molecule, for example an oligonucleotide of about 2, 3, 4, 5, 6, 7, 8, 9, 10, 11, 12, 13, 14, 15, 16, 17, 18, 19, 20, 21, 22, 23, 24, 25, or more nucleotides in length.

A direct linkage includes any linkage wherein a linker moiety is not required. In one embodiment, a direct linkage includes a chemical or a physical interaction wherein the two moieties, the siRNA and the carrier polymer, interact such that they are attracted to each other. Examples of direct interactions include non-covalent interactions, hydrophobic/hydrophilic, ionic (e.g., electrostatic, coulombic attraction, ion-dipole, charge-transfer), Van der Waals, or hydrogen bonding, and chemical bonding, including the formation of a covalent bond. Accordingly, in one embodiment, the siRNA and the carrier polymer are not linked via a linker, e.g., they are directly linked. In a further embodiment, the siRNA and the carrier polymer are electrostatically associated with each other.

A viral-mediated delivery mechanism may also be employed to deliver siRNAs to cells in vitro and in vivo as described in Xia, H. et al. (2002) Nat Biotechnol 20(10): 1006). Plasmid- or viral-mediated delivery mechanisms of shRNA may also be employed to deliver shRNAs to cells in vitro and in vivo as described in Rubinson, D. A., et al. ((2003) Nat. Genet. 33:401-406) and Stewart, S. A., et al. ((2003) RNA 9:493-501). Other methods of introducing siRNA molecules of the present invention to target cells, such as dendritic cells, include a variety of art-recognized techniques including, but not limited to, calcium phosphate or calcium chloride co-precipitation, DEAE-dextran-mediated transfection, lipofection, or electroporation as well as a number of commercially available transfection kits (e.g., OLIGOFECTAMINE®. Reagent from Invitrogen, Carlsbad, Calif.; LIPOFECTAMINE®. 2000 from Invitrogen, Carlsbad, Calif.; I-FECT™. from Neuromics, Bloomington, Minn.; JetSI/DOPE (Avanti Polar Lipids, Alabaster, Ala.) (see, e.g. Sui, G. et al. (2002) Proc. Natl. Acad. Sci. USA 99:5515-5520; Calegari, F. et al. (2002) Proc. Natl. Acad. Sci. 99:14236-40; J-M Jacque, K. Triques and M. Stevenson (2002) Nature 418:435-437; and Elbashir, S. M et al. (2001) supra). Suitable methods for transfecting a target cell, e.g., a neuronal cell, a macrophage, an epithelial cell, can also be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals. The efficiency of transfection may depend on a number of factors, including the cell type, the passage number, the confluency of the cells as well as the time and the manner of formation of siRNA- or shRNA-liposome complexes (e.g., inversion versus vortexing). These factors can be assessed and adjusted without undue experimentation by one with ordinary skill in the art.

The siRNAs or shRNAs of the invention, may be introduced along with components that perform one or more of the following activities: enhance uptake of the siRNA, by the target cell, inhibit annealing of single strands, stabilize single strands, or otherwise facilitate delivery to the target cell and increase inhibition of NLRC5 gene expression.

The siRNA may also be directly introduced into the target cell, or introduced extracellularly into a cavity, interstitial space, into the circulation of an organism, introduced orally, introduced nasally, introduced intracranially or may be introduced by bathing a cell or organism in a solution containing the siRNA. The siRNA may also be introduced into cells via topical application to a mucosal membrane or dermally. Vascular or extravascular circulation, the blood or lymph system, and the cerebrospinal fluid are also sites where the agents may be introduced.

As used herein, the term “vector” refers to a nucleic acid molecule capable of transporting another nucleic acid to which it has been linked. One type of vector is a “plasmid”, which refers to a circular double stranded DNA loop into which additional nucleic acid segments can be ligated. Another type of vector is a viral vector, wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors”, or more simply “expression vectors.” In general, expression vectors of utility in recombinant DNA techniques are often in the form of plasmids. In some embodiments, “plasmid” and “vector” are used interchangeably as the plasmid is the most commonly used form of vector. However, the invention is intended to include all other forms of expression vectors, such as viral vectors (e.g., replication defective retroviruses, lentiviruses, adenoviruses and adeno-associated viruses), which serve equivalent functions. In a embodiment, lentiviruses are used to deliver one or more siRNA molecule of the present invention to a cell.

Within an expression vector, “operably linked” is intended to mean that the nucleotide sequence of interest is linked to the regulatory sequence(s) in a manner which allows for expression of the nucleotide sequence (e.g., in an in vitro transcription/translation system or in a target cell when the vector is introduced into the target cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). Furthermore, the siRNAs may be delivered by way of a vector comprising a regulatory sequence to direct synthesis of the siRNAs of the invention at specific intervals, or over a specific time period. It will be appreciated by those skilled in the art that the design of the expression vector can depend on such factors as the choice of the target cell, the level of expression of siRNA desired, and the like.

The expression vectors of the invention can be introduced into target cells to thereby produce siRNA molecules of the present invention. In one embodiment, a DNA template, for example, a DNA template encoding NLRC5 gene may be ligated into an expression vector under the control of RNA polymerase III (Pol III), and delivered to a target cell. Pol III directs the synthesis of small, noncoding transcripts which 3′ ends are defined by termination within a stretch of 4-5 thymidines. Accordingly, DNA templates may be used to synthesize, in vivo, both sense and antisense strands of siRNAs which effect RNAi (Sui, et al. (2002) PNAS 99(8):5515).

The expression vectors of the invention may also be used to introduce shRNA into target cells. The useful expression vectors also be inducible vectors, such as tetracycline (see, e.g., Wang et al. Proc Natl Acad Sci U.S.A. 100: 5103-5106, 2003) or ecdysone inducible vectors (e.g., from Invitrogen) known to one skilled in the art.

As used herein, the term “target cell” is intended to refer to any cell in the body into which an siRNA molecule of the invention, including a recombinant expression vector encoding an siRNA of the invention, is being or has been introduced. The terms “target cell” and “host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. In one embodiment, a target cell is a mammalian cell, e.g., a human cell.

It is known that depending upon the expression vector and transfection technique used, only a small fraction of cells may effectively uptake the siRNA molecule. In order to identify and select these cells, antibodies against a cellular target can be used to determine transfection efficiency through immunofluorescence. Typical cellular targets include those which are present in the host cell type and whose expression is relatively constant, such as Lamin A/C. Alternatively, co-transfection with a plasmid containing a cellular marker, such as a CMV-driven EGFP-expression plasmid, luciferase, metalloprotease, BirA, B-galactosidase and the like may also be used to assess transfection efficiency. Cells that have been transfected with the siRNA molecules can then be identified by routine techniques such as immunofluorescence, phase contrast microscopy or fluorescence microscopy, for example.

In certain cases, depending on the abundance and the life-time (or turnover) of the targeted protein, a knock-down phenotype, e.g., a phenotype associated with siRNA inhibition of the target NLRC5 gene expression may become apparent after 1 to 3 days, or even later. In cases where no phenotype is observed, depletion of the protein may be observed by immunofluorescence or Western blotting. If the protein is still abundant after 3 days, cells can be split and transferred to a fresh 24-well plate for re-transfection. In one embodiment the depletion of the expression of the allele is monitored using RNA quantification techniques capable of easily distinguishing the expression of the disease allele from the expression of healthy allele.

If no knock-down of the targeted protein is observed, it may be desirable to analyze whether the target mRNA was effectively destroyed by the transfected siRNA duplex. Two days after transfection, total RNA can be prepared, reverse transcribed using a target-specific primer, and PCR-amplified with a primer pair covering at least one exon-exon junction in order to control for amplification of pre-mRNAs. RT-PCR of a non-targeted mRNA is also needed as control. Effective depletion of the mRNA yet undetectable reduction of target protein may indicate that a large reservoir of stable protein may exist in the cell. Multiple transfection in sufficiently long intervals may be necessary until the target protein is finally depleted to a point where a phenotype may become apparent.

The dose of the siRNA will be in an amount necessary to effect RNA interference, e.g., post translational gene silencing (PTGS), of the particular target gene, thereby leading to inhibition of target gene expression or inhibition of activity or level of the protein encoded by the target gene. Assays to determine expression of the target allele, are known in the art. For example, reduced levels of target gene mRNA may be measured by in situ hybridization (Montgomery et al., (1998) Proc. Natl. Acad. Sci., USA 95:15502-15507) or Northern blot analysis (Ngo, et al. (1998)) Proc. Natl. Acad. Sci., USA 95:14687-14692). In one embodiment, target gene transcription is measured using quantitative real-time PCR (Gibson et al., Genome Research 6:995-1001, 1996; Heid et al., Genome Research 6:986-994, 1996).

### V. Methods of Enhancing Immune Response Against Microbial Infection or Inflammation-Associated Disease

The active siRNAs of the present invention are administered in prophylactically or therapeutically effective amounts. A prophylactically or therapeutically effective amount means that amount necessary, at least partly, to attain the desired effect, or to delay the onset of, inhibit the progression of, or halt altogether, the onset or progression of the particular microbial infection or inflammation-associated disease being treated or prevented. Such amounts will depend, of course, on the particular condition being treated, the severity of the condition and individual patient parameters including age, physical condition, size, weight and concurrent treatment. These factors are well known to those of ordinary skill in the art and can be addressed with no more than routine experimentation. It is usual generally that a maximum dose be used, that is, the highest safe dose according to sound medical judgment. It will be understood by those of ordinary skill in the art, however, that a lower dose or tolerable dose may be administered for medical reasons, psychological reasons or for virtually any other reasons.

In one aspect, the invention provides a method for preventing in a subject, an infectious disease or inflammation-associated disorder, by administering to the subject one or more therapeutic agents, e.g., the siRNAs as described herein. For example, the siRNAs described herein may be used as antimicrobial agents for an individual at risk for contracting an antimicrobial infection or at risk for having an inflammation-associated disorder. Subjects at risk for an infectious disease or inflammation-associated disorder, can be identified by, for example, travel history, travel plans, lifestyle, immune state, pregnancy, old age or any known risk factors for an infectious disease or inflammation-associated disorder.

Administration of a prophylactic agent can occur prior to the manifestation of symptoms characteristic of a microbial infection or inflammation-associated disorder, such that the infection or disorder is prevented or, alternatively, delayed in its progression. Any mode of administration of the therapeutic agents of the invention, as described herein or as known in the art, including parenteral, intranasal or intracranial administration of the siRNAs of the instant invention, for example, may be utilized for the prophylactic treatment of an infection or disorder.

Formulations of the active compounds as described herein (e.g., an siRNA) may be administered to a subject at risk for a microbial infection or inflammation-associated disease.

In one embodiment, the compositions comprising the siRNA (and in some cases a carrier polymer) may be administered prior to exposure to the infectious agent. In vitro experiments illustrate that the antiviral state induced by introduced duplex siRNAs can last for three weeks. Therefore, in one embodiment, an siRNA-based antiviral need not be applied before encounter with an infectious agent. Accordingly, in another embodiment, the prophylactic effect of the siRNA is prolonged, e.g., lasts for at least one week, in one embodiment two or more weeks. In another embodiment, the compositions comprising the siRNA may be administered, e.g., parenterally or intranasally, at intervals, e.g., one or more times per week, or one or more times per month, rather than directly prior to exposure to an infectious agent.

In another aspect, the invention provides a method for treating in a subject, an antimicrobial infection or inflammation-associated disorder, by administering to the subject one of more therapeutic agents, e.g., the siRNAs as described herein. For example, the siRNAs described herein may be administered to a subject infected by a microbe (for example a pathogenic microbe) or inflammation-associated disorder or at risk for being infected by a pathogenic microbe or at risk for developing an inflammation-associated disorder.

The term “therapeutically effective amount” refers to an amount that is sufficient to effect a therapeutically or prophylactically significant reduction in an antimicrobial infection or inflammation-associated disorder when administered to a typical subject who is either infected with the microbe or who is at risk of being infected with the microbe, or a typical subject who has an inflammation-associated disorder or who is at risk for having an inflammation-associated disorder. In aspects involving administration of an antimicrobial or immune response-enhancing siRNA to a subject, typically the siRNA, formulation, or composition should be administered in a therapeutically effective amount.

Generally, at intervals to be determined by the prophylaxis or treatment of pathogenic states, doses of active component will be from about 0.01 mg/kg per day to 1000 mg/kg per day. Small doses (0.01-1 mg) may be administered initially, followed by increasing doses up to about 1000 mg/kg per day. In the event that the response in a subject is insufficient at such doses, even higher doses (or effective higher doses by a different, more localized delivery route) may be employed to the extent patient tolerance permits. Multiple doses per day can be contemplated to achieve appropriate systemic levels of compounds.

Another aspect of the invention pertains to methods of modulating gene expression or protein activity, for example, cellular gene expression or activity and/or expression or activity of a gene or sequence of NLRC5 gene expression or protein activity in order to treat a microbial infection or inflammation-associated disorder. Accordingly, in an exemplary embodiment, the modulatory method of the invention involves contacting a cell with a therapeutic agent (e.g., one or more siRNAs, e.g., one or more siRNAs targeting a cellular gene or sequence), such that expression of the target gene or genes is prohibited. These methods can be performed in vitro, for example by culturing the cell, or in vivo, for example by administering the siRNA to a subject infected with antimicrobial infection or at risk of infection with a microbe, or a subject having or at risk for an inflammation-associated disorder.

The prophylactic or therapeutic pharmaceutical compositions of the invention can contain other pharmaceuticals, in conjunction with a vector according to the invention, when used to therapeutically treat microbial-mediated or inflammation associated disease, and can also be administered in combination with other pharmaceuticals used to treat microbial infection or inflammation-associated disease. For example, the prophylactic or therapeutic pharmaceutical compositions of the invention can also be used in combination with other pharmaceuticals that treat or alleviate symptoms of microbial infection. The prophylactic or therapeutic pharmaceutical compositions of the invention can be used in combination with practices used in supportive care of microbial infection, such as airway management, respiratory support, intravenous fluids, or any combination thereof.

In certain embodiments, there is a method of inhibiting expression of NLRC5 mRNA, or mutant or variant thereof, comprising administering to a subject an effective amount of siRNA comprising a sense RNA strand and an antisense RNA strand, or a single RNA strand, wherein the sense and the antisense RNA strands, or the single RNA strand, form an RNA duplex, and wherein the RNA strand comprises a nucleotide sequence identical to a target sequence of about 15 to about 30 (for example of about 19 to about 25) contiguous nucleotides in NLRC5 mRNA, or an alternative splice form, mutant or cognate thereof, is degraded.

In some embodiments, there is a method of enhancing immune response against microbial infection or inflammation-associated disease in a subject, comprising administering to a subject an effective amount of an siRNA comprising a sense RNA strand and an antisense RNA strand, or a single RNA strand, wherein the sense and the antisense RNA strands, or the single RNA strand, form an RNA duplex, and wherein the RNA strand comprises a nucleotide sequence identical to a target sequence of about 15 to about 30 contiguous nucleotides in NLRC5 mRNA, or mutant or variant thereof.

In some embodiments, there is a method of preventing microbial infection or inflammation-associated disease in a subject, comprising administering to a subject an effective amount of an siRNA comprising a sense RNA strand and an antisense RNA strand, or a single RNA strand, wherein the sense and the antisense RNA strands, or the single RNA strand, form an RNA duplex, and wherein the RNA strand comprises a nucleotide sequence identical to a target sequence of about 15 to about 30 contiguous nucleotides in NLRC5 mRNA, or mutant or variant thereof.

The term “preventing” as used herein refers to preventing microbial infection in an individual susceptible for infection or preventing one or more symptoms of an inflammation-associated disorder. Whether effective prevention is achieved can be tested using routine detection methods including, but not limited to, IgM ELISA, IgG ELISA, IgA ELISA, blocking ELISA, IgG by indirect fluorescent antibody (IFA), microsphere immunoassay, Plaque Reduction Neutralization Test (PRNT), RT-PCR, Real Time RT-PCR, quantitative RT-PCR, TAQMAN®. (Roche) assay, Nucleic Acid Sequence Based Amplification (NASBA; BioMerieux, Marcy l'Etoile, France) or any combination thereof, using blood, serum, cerebral spinal fluid or any combination thereof.

The invention further provides a method of treating microbial disease or inflammation-associated disease in a subject comprising administering to the subject, such as a mammal, for example a canine, such as a dog, or a primate, such as a human, an effective amount of the siRNAs of the present invention comprising a sense RNA strand and an antisense RNA strand, or a single RNA strand, wherein the sense and an antisense RNA strands, or the single RNA strand, form an RNA duplex, and wherein the RNA strand comprises a nucleotide sequence identical to a target sequence of about 19 to about 25 contiguous nucleotides in NLRC5 mRNA, or mutant or variant thereof.

In one embodiment, the siRNA used in the methods of the invention, are actively taken up by cells in vivo following administration, illustrating efficient in vivo delivery of the siRNAs used in the methods of the invention.

Other strategies for delivery of the siRNAs used in the methods of the invention, can also be employed, such as, for example, delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the siRNAs or shRNAs of the invention, using a basic peptide by conjugating or mixing the siRNA with a basic peptide, e.g., a fragment of a TAT peptide, mixing with cationic lipids or formulating into particles.

In one embodiment, the dsRNA, such as siRNA or shRNA, is delivered using an inducible vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif.) can be used.

In one embodiment, the siRNAs used in the methods of the invention, can be introduced into cells, e.g., cultured cells, which are subsequently transplanted into the subject by, e.g., transplanting or grafting, or alternatively, can be obtained from a donor (i.e., a source other than the ultimate recipient), and applied to a recipient by, e.g., transplanting or grafting, subsequent to administration of the siRNAs of the invention to the cells. Alternatively, the siRNAs of the invention can be introduced directly into the subject in such a manner that they are directed to and taken up by the target cells and regulate or promote RNA interference of the target NLRC5 gene. The siRNAs of the invention may be delivered singly, or in combination with other siRNAs. The siRNAs of the invention may also be administered in combination with other pharmaceutical agents that are used to treat NLRC5 infection.

In specific embodiments, the NLRC5 targeting siRNAs are designed so as to maximize the uptake of the antisense (guide) strand of the siRNA into RNA-induced silencing complex (RISC) and thereby maximize the ability of RISC to target NLRC5 mRNA for degradation. This can be accomplished by looking for sequences that have the lowest free energy of binding at the 5′-terminus of the antisense strand. The lower free energy would lead to an enhancement of the unwinding of the 5′-end of the antisense strand of the siRNA duplex, thereby ensuring that the antisense strand will be taken up by RISC and direct the sequence-specific cleavage of the NLRC5 mRNA.

“RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double stranded fragments termed siRNAs. siRNAs are incorporated into a protein complex that recognizes and cleaves target mRNAs. RNAi can also be initiated by introducing nucleic acid molecules, e.g., synthetic siRNAs, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease may be of at least 30%, 40%, 50%, 60%, 70%, 80%, 90%, 95% or 99% or more as compared to the expression of a target gene or the activity or level of the protein encoded by a target gene which has not been targeted by an siRNA.

The target gene or sequence of the siRNA is designed to be substantially homologous to the target sequence, or a fragment thereof. As used herein, the term “homologous” is defined as being substantially identical, sufficiently complementary, or similar to the target NLRC5 mRNA, or a fragment thereof, to effect RNA interference of the target. In addition to native RNA molecules, RNA suitable for inhibiting or interfering with the expression of a target sequence include RNA derivatives and analogs. In one embodiment, the siRNA is identical to its target allele so as to prevent its interaction with the normal allele.

The siRNAs used in the methods of the invention typically target only one sequence. In one embodiment, a mixture of siRNAs designed to inhibit expression of one or more NLRC5 sequences are used in combination. Each of the siRNAs, can be screened for potential off-target effects may be analyzed using, for example, expression profiling. Such methods are known to one skilled in the art and are described, for example, in Jackson et al. Nature Biotechnology 6:635-637, 2003. In addition to expression profiling, one may also screen the potential target sequences for similar sequences in the sequence databases to identify potential sequences which may have off-target effects. For example, according to Jackson et al. (Id.) 15, or perhaps as few as 11 contiguous nucleotides, of sequence identity are sufficient to direct silencing of non-targeted transcripts. Therefore, one may initially screen the proposed siRNAs to avoid potential off-target silencing using the sequence identity analysis by any known sequence comparison methods, such as Basic Local Alignment Search Tool (BLAST) from NCBI (U.S. National Institutes of Health information database).

siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues may be used, such as a phosphorothioate linkage. The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatives with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases may also be modified. Any modified base useful for inhibiting or interfering with the expression of a target sequence may be used. For example, halogenated bases, such as 5-bromouracil and 5-iodouracil can be incorporated. The bases may also be alkylated, for example, 7-methylguanosine can be incorporated in place of a guanosine residue. Non-natural bases that yield successful inhibition can also be incorporated.

In one embodiment, siRNA modifications include 2′-deoxy-2′-fluorouridine or locked nucleic acid (LAN) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology.

In certain embodiments of the invention, NLRC5 is targeted with one or more siRNA molecules. In specific embodiments, an individual with or suspected of having a microbial infection or inflammation-associated disease is provided with one or more siRNAs directed against NLRC5. In particular embodiments, an individual with cancer, suspected of having cancer, at a high risk for cancer, or susceptible to cancer is administered one or more siRNA molecules directed against NLRC5. In specific embodiments a cancer cell or a cell suspected of being or becoming cancerous is provided with one or more siRNAs directed against NLRC5. In particular embodiments, an individual with cancer, suspected of having cancer, at a high risk for cancer, or susceptible to cancer is administered one or more siRNA molecules directed against NLRC5.

The present invention includes methods and compositions for introducing multiple siRNAs targeting different regions of a gene that typically can greatly improve the likelihood that the expression of the target gene will be reduced. In certain embodiments, different candidate siRNAs or siRNAs do not interfere with the activities of others in the mixture and, in some cases there may be some synergy between the siRNAs. This is applicable not only to siRNAs but to DNA constructs designed to express siRNAs (Brummelkamp, 2002). Certain embodiments of the invention alleviate the need to screen or optimize candidate siRNAs. To determine the functionality of a candidate siRNA, it may be screened, verified, and/or optimized, in certain aspects. As used herein, a “candidate siRNA” is an siRNA that has not been tested for its functionality as an siRNA. It is also contemplated that siRNAs may be single or double stranded RNA molecules.

In some embodiments of the invention, methods are employed wherein multiple therapeutic RNAs are employed, each of which reduce the expression of a target gene to some degree, as well as the presence of some dsRNAs, which do not effect target gene expression, may be administered as a pool without interference between members of the pool and may result in an additive or synergistic reduction in target gene expression. Thus, the present invention is directed to compositions and methods involving generation and utilization of pools or mixtures of small, double-stranded RNA molecules that effect, trigger, or induce RNAi more effectively. RNAi is mediated by an RNA-induced silencing complex (RISC), which associates (specifically binds one or more RISC components) with dsRNA pools of the invention and guides the dsRNA to its target mRNA through base-pairing interactions. Once the dsRNA is base-paired with its mRNA target, nucleases cleave the mRNA.

In certain embodiments of the invention, one or more siRNAs or dsRNAs can be introduced into a cell to activate the RNAi pathway. In other embodiments, various individual siRNAs or dsRNAs with different sequences may be co-transfected simultaneously to effectively produce a pool or mixture of dsRNAs within a transfected cell(s). The effects of multiple siRNAs are typically additive and may be synergistic in some cases.

In some embodiments, the invention concerns a siRNA or dsRNA that is capable of triggering RNA interference, a process by which a particular RNA sequence is destroyed (also referred to as gene silencing). In certain embodiments, siRNA are dsRNA molecules that are 100 bases or fewer in length (or have 100 basepairs or fewer in its complementarity region). A dsRNA may be 15, 16, 17, 18, 19, 20, 21, 22, 23, 24, 25, 26, 27, 28, 29, 30, 40, 50, 60, 70, 80, 90, 100 110, 120, 130, 140, 150, 160, 170, 180, 190, 200, 225, 250, 275, 300, 325, 350. 375, 400, 450, 500, 550, 600, 650, 700, 750, 800, 850, 900, 950, or 1000 nucleotides or more in length. In certain embodiments, siRNA may be approximately 21 to 25 nucleotides in length. In some cases, it has about a two nucleotide 3′ overhang and a 5′ phosphate. The particular RNA sequence is targeted as a result of the complementarity between the dsRNA and the particular RNA sequence. It will be understood that siRNA or dsRNA of the invention can effect at least about a 20, 30, 40, 50, 60, 70, 80, 90 percent or more reduction of expression of a targeted RNA in a cell. dsRNA of the invention (the term “dsRNA” will be understood to include “siRNA” and/or “candidate siRNA”) is distinct and distinguishable from antisense and ribozyme molecules by virtue of the ability to trigger RNAi. Structurally, dsRNA molecules for RNAi comprise at least one region of complementarity within the RNA molecule. The complementary (also referred to as “complementarity”) region comprises at least or at most about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous bases. In some embodiments, long dsRNA are employed in which “long” refers to dsRNA that are 1000 bases or longer (or 1000 basepairs or longer in complementarity region). The term “dsRNA” includes “long dsRNA”, “intermediate dsRNA” or “small dsRNA” (lengths of 2 to 100 bases or basepairs in complementarity region) unless otherwise indicated.

It is specifically contemplated that a dsRNA may be a molecule comprising two separate RNA strands in which one strand has at least one region complementary to a region on the other strand. Alternatively, a dsRNA includes a molecule that is single stranded yet has at least one complementarity region as described above (see Sui et al., 2002 and Brummelkamp et al., 2002 in which a single strand with a hairpin loop is used as a dsRNA for RNAi). For convenience, lengths of dsRNA may be referred to in terms of bases, which simply refers to the length of a single strand or in terms of basepairs, which refers to the length of the complementarity region. It is specifically contemplated that embodiments discussed herein with respect to a dsRNA comprised of two strands are contemplated for use with respect to a dsRNA comprising a single strand, and vice versa. In a two-stranded dsRNA molecule, the strand that has a sequence that is complementary to the targeted mRNA is referred to as the “antisense strand” and the strand with a sequence identical to the targeted mRNA is referred to as the “sense strand.” Similarly, with a dsRNA comprising only a single strand, it is contemplated that the “antisense region” has the sequence complementary to the targeted mRNA, while the “sense region” has the sequence identical to the targeted mRNA. Furthermore, it will be understood that sense and antisense region, like sense and antisense strands, are complementary (i.e., can specifically hybridize) to each other.

Strands or regions that are complementary may or may not be 100% complementary (“completely or fully complementary”). It is contemplated that sequences that are “complementary” include sequences that are at least about 50% complementary, and may be at least about 50%, 60%, 70%, 80%, or 90% complementary. In the range of about 50% to 70% complementarity, such sequences may be referred to as “very complementary,” while the range of greater than about 70% to less than complete complementarity can be referred to as “highly complementary.” Unless otherwise specified, sequences that are “complementary” include sequences that are “very complementary,” “highly complementary,” and “fully complementary.”It is also contemplated that any embodiment discussed herein with respect to “complementary” strands or region can be employed with specifically “fully complementary,” “highly complementary,” and/or “very complementary” strands or regions, and vice versa. Thus, it is contemplated that in some instances, as demonstrated in the Examples, that siRNA generated from sequence based on one organism may be used in a different organism to achieve RNAi of the cognate target gene. In other words, siRNA generated from a dsRNA that corresponds to a human gene may be used in a mouse cell if there is the requisite complementarity, as described above. Ultimately, the requisite threshold level of complementarity to achieve RNAi is dictated by functional capability.

It is specifically contemplated that there may be mismatches in the complementary strands or regions. Mismatches may number at most or at least 1, 2, 3, 4, 5, 6, 7, 8, 9, 10, 11, 12, 13, 14, 15, 16, 17, 18, 19, 20, 21, 22, 23, 24, 25 residues or more, depending on the length of the complementarity region.

The single RNA strand or each of two complementary double strands of a dsRNA molecule may be of at least or at most the following lengths: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 31, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 or more (including the full-length of a particular's gene's mRNA without the poly-A tail) bases or basepairs. If the dsRNA is comprised of two separate strands, the two strands may be the same length or different lengths. If the dsRNA is a single strand, in addition to the complementarity region, the strand may have 1, 2, 3, 4, 5, 6, 7, 8, 9, 10, 11, 12, 13, 14, 15, 16, 17, 18, 19, 20, 21, 22, 23, 24, 25, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more bases on either or both ends (5′ and/or 3′) or as forming a hairpin loop between the complementarity regions.

In certain embodiments of the invention, there is an siRNA comprising a sense region and an antisense region, wherein said sense region and said antisense region together form a duplex region, said antisense region and said sense region are each 15-30 nucleotides in length and said antisense region comprises a sequence that is at least 75%, 80%, 85%, 90%, or 95% complementary to a sequence selected from the group consisting of SEQ ID NO:2, SEQ ID NO:4, SEQ ID NO:5, SEQ ID NO:6, SEQ ID NO:7, and SEQ ID NO:12. In particular cases of the invention, there is an siRNA comprising a sense region and an antisense region, wherein said sense region and said antisense region together form a duplex region, said antisense region and said sense region are each 15-30, 19-30, 19-25, or 20-25 nucleotides, for example, in length and said antisense region comprises a sequence that is at least 90%, at least 95%, at least 97%, at least 99%, or 100% complementary to a sequence selected from the group consisting of SEQ ID NO:2, SEQ ID NO:4, SEQ ID NO:5, SEQ ID NO:6, SEQ ID NO:7, and SEQ ID NO:12. In certain embodiments of the invention, there is an siRNA comprising a sense region and an antisense region, wherein said sense region and said antisense region together form a duplex region, said antisense region and said sense region are each 15-30 nucleotides in length and said antisense region comprises a sequence that is between 75% to 85%, 80%-90%, 85%-95%, 90%-95%, or 90%-97% complementary to a sequenced selected from the group consisting of SEQ ID NO:2, SEQ ID NO:4, SEQ ID NO:5, SEQ ID NO:6, SEQ ID NO:7, and SEQ ID NO:12.

Furthermore, it is contemplated that siRNA or the longer dsRNA template may be labeled. The label may be fluorescent, radioactive, enzymatic, or colorimetric, for example. It is contemplated that a dsRNA may have one label attached to it or it may have more than one label attached to it. When more than one label is attached to a dsRNA, the labels may be the same or be different. If the labels are different, they may appear as different colors when visualized. The label may be on at least one end and/or it may be internal. Furthermore, there may be a label on each end of a single stranded molecule or on each end of a dsRNA made of two separate strands. The end may be the 3′ and/or the 5′ end of the nucleic acid. A label may be on the sense strand or the sense end of a single strand (end that is closer to sense region as opposed to antisense region), or it may be on the antisense strand or antisense end of a single strand (end that is closer to antisense region as opposed to sense region). In some cases, a strand is labeled on a particular nucleotide (G, A, U, or C). When two or more differentially colored labels are employed, fluorescent resonance energy transfer (FRET) techniques may be employed to characterize the dsRNA.

Labels contemplated for use in several embodiments may be non-radioactive. In many embodiments of the invention, the labels are fluorescent, though they may be enzymatic, radioactive, or positron emitters. Fluorescent labels that may be used include, but are not limited to, BODIPY, Alexa Fluor, fluorescein, Oregon Green, tetramethylrhodamine, Texas Red, rhodamine, cyanine dye, or derivatives thereof, for example. The labels may also more specifically be Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5, DAPI, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, TET, Tetramethylrhodamine, and/or Texas Red, for example. A labeling reagent is a composition that comprises a label and that can be incubated with the nucleic acid to effect labeling of the nucleic acid under appropriate conditions. In some embodiments, the labeling reagent comprises an alkylating agent and a dye, such as a fluorescent dye. In some embodiments, a labeling reagent comprises an alkylating agent and a fluorescent dye such as Cy3, Cy5, or fluorescein (FAM). In still further embodiments, the labeling reagent is also incubated with a labeling buffer, which may be any buffer compatible with physiological function (i.e., buffers that is not toxic or harmful to a cell or cell component) (termed “physiological buffer”).

In some embodiments of the invention, a dsRNA has one or more non-natural nucleotides, such as a modified residue or a derivative or analog of a natural nucleotide. Any modified residue, derivative or analog may be used to the extent that it does not eliminate or substantially reduce (by at least 50%) RNAi activity of the dsRNA.

A person of ordinary skill in the art is well aware of achieving hybridization of complementary regions or molecules. Such methods typically involve heat and slow cooling of temperature during incubation.

Any cell that undergoes RNAi can be employed in methods of the invention. The cell may be a eukaryotic cell, mammalian cell such as a primate, rodent, rabbit, canine, feline, equine, or human cell, for example.

In some embodiments of the invention, there are methods of reducing the expression of a target gene in a cell. Such methods involve the compositions described above, including the embodiments described for RNase III, dsRNA, and siRNA. In various embodiments of the invention, reduction or elimination of expression of one or more target genes may be accomplished by the a) obtaining one or more siRNA or dsRNA molecules corresponding one or more target genes and b) transfecting the respective siRNA or dsRNA molecules corresponding to the one or more target genes into a cell.

In some methods of the invention, siRNA and/or candidate siRNA molecules or template nucleic acids may be isolated or purified prior to their being used in a subsequent step. siRNA and/or candidate siRNA molecules may be isolated or purified prior to introduction into a cell. “Introduction” into a cell includes known methods of transfection, transduction, infection and other methods for introducing an expression vector or a heterologous nucleic acid into a cell. A template nucleic acid or amplification primer may be isolated or purified prior to it being transcribed or amplified. Isolation or purification can be performed by a number of methods known to those of skill in the art with respect to nucleic acids. In some embodiments, a gel, such as an agarose or acrylamide gel, is employed to isolate the siRNA and/or candidate siRNA.

In some methods of the invention dsRNA is obtained by transcribing each strand of the dsRNA from one or more cDNA (or DNA or RNA) encoding the strands in vitro. It is contemplated that a single template nucleic acid molecule may be used to transcribe a single RNA strand that has at least one region of complementarity (and is thus double-stranded under conditions of hybridization) or it may be used to transcribe two separate complementary RNA molecules. Alternatively, more than one template nucleic acid molecule may be transcribed to generate two separate RNA strands that are complementary to one another and capable of forming a dsRNA.

Additional methods involve isolating the transcribed strand(s) and/or incubating the strand(s) under conditions that allow the strand(s) to hybridize to their complementary strands (or regions if a single strand is employed).

Nucleic acid templates may be generated by a number of methods well known to those of skill in the art. In some embodiments the template, such as a cDNA, is synthesized through amplification or it may be a nucleic acid segment in or from a plasmid that harbors the template.

In various embodiments, siRNAs are encoded by expression constructs. The expression constructs may be obtained and introduced into a cell. Once introduced into the cell the expression construct is transcribed to produce various siRNAs. Expression constructs include nucleic acids that provide for the transcription of a particular nucleic acid. Expression constructs include plasmid DNA, linear expression elements, circular expression elements, viral expression constructs, and the like, all of which are contemplated as being used in the compositions and methods of the present invention. In certain embodiments at least about 2, 3, 4, 5, 6, 7, 8, 9, 10 or more siRNA molecules are encoded by a single expression construct. Expression of the siRNA molecules may be independently controlled by at least about 2, 3, 4, 5, 6, 7, 8, 9, 10 or more promoter elements. In certain embodiments, at least about 2, 3, 4, 5, 6, 7, 8, 9, 10 or more expression constructs may introduced into the cell. Each expression construct may encode 1, 2, 3, 4, 5, 6, 7, 8, 9, 10 or more siRNA molecules. In certain embodiments siRNA molecules may be encoded as expression domains. Expression domains include a transcription control element, which may or may not be independent of other control or promoter elements; a nucleic acid encoding an siRNA; and optionally a transcriptional termination element. In other words, an siRNA cocktail or pool may be encoded by a single or multiple expression constructs. In particular embodiments the expression construct is a plasmid expression construct.

Other methods of the invention also concern transcribing a strand or strands of a dsRNA using a promoter that can be employed in vitro or outside a cell, such as a prokaryotic promoter. In some embodiments, the prokaryotic promoter is a bacterial promoter or a bacteriophage promoter. It is specifically contemplated that dsRNA strands are transcribed with SP6, T3, or T7 polymerase.

### VI. Pharmaceutical Compositions

In certain embodiments, the invention provides a pharmaceutical composition comprising at least one siRNA and a pharmaceutically acceptable carrier, wherein the siRNA comprises a sense RNA strand and an antisense RNA strand, or a single RNA strand, wherein the sense and the antisense RNA strands, or the single RNA strand, form an RNA duplex, and wherein the RNA strand comprises a nucleotide sequence identical to a target sequence of about 19 to about 25 contiguous nucleotides in NLRC5 mRNA, or an alternative splice form, mutant or cognate thereof.

A pharmaceutically acceptable carrier refers to generally available and known pharmaceutical carriers and diluents. The formulation of such compositions is well known to persons skilled in this field. Suitable pharmaceutically acceptable carriers and/or diluents include any and all solvents, dispersion media, fillers, solid carriers, aqueous solutions, coatings, antibacterial and anti fungal agents, isotonic, and absorption enhancing or delaying agents, activity enhancing or delaying agents and the like. The use of such media and agents for pharmaceutically active substances is well known in the art, and it is described, by way of example, in Remington's Pharmaceutical Sciences, 18th Edition, Mack Publishing Company, Pennsylvania, USA. Except insofar as any conventional carrier and/or diluent is incompatible with the active ingredient, use thereof in the pharmaceutical compositions of the present invention is contemplated. Supplementary active ingredients including agents having antiviral or antimicrobial activity can also be incorporated into the compositions of this invention.

The active compositions of the present invention may include classic pharmaceutical preparations. Administration of these compositions according to the present invention will be via any common route so long as the target tissue is available via that route. The routes of administration will vary, naturally, with the location and nature of the infection, and the dosage required for prophylactic or therapeutic efficacy. The routes of administration include, e.g., intradermal, transdermal, transmucosal, parenteral, intracranial, intravenous, intramuscular, intranasal, intracerebrospinal, subcutaneous, percutaneous, intratracheal, intraperitoneal, intratumoral, perfusion, lavage, direct injection, and oral administration and formulation. In the present invention, intracranial, intranasal or intravenous administration are exemplary embodiments. Administration may be by injection or infusion. The methods of this invention, generally speaking, may be practiced using any mode of administration that is medically acceptable, meaning any mode that produces prophylactic or therapeutic levels of the active component of the invention without causing clinically unacceptable adverse effects.

For methods of performing intracranial administration, please see Kruse et al. (J. Neuro-Oncol., 19:161-168, 1994), specifically incorporated by reference. Such compositions would normally be administered as pharmaceutically acceptable compositions.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Parenteral modes of administration for the present invention include intravenous, intracranial, intramuscular, intradermal, subcutaneous, and oral (e.g., inhalation) administrations. Solutions or suspensions used for parenteral, intracranial, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

In certain embodiments, the pharmaceutical compositions may be delivered by intranasal sprays, inhalation, and/or other aerosol delivery vehicles. For aerosol delivery vehicles, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch. Methods for delivering genes, nucleic acids, and peptide compositions directly to the lungs via nasal aerosol sprays has been described e.g., in U.S. Pat. No. 5,756,353 and U.S. Pat. No. 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroethylene support matrix is described in U.S. Pat. No. 5,780,045 (specifically incorporated herein by reference in its entirety). As previously shown, intranasal administration (Mathison et al, J. Drug Target, 5 (6):415-441 (1998); Chou et al, Biopharm Drug Dispos. 18 (4):335-46 (1997); Draghia et al, Gene Therapy 2:418-423 (1995)) may enable the direct entry of viruses and macromolecules into the CSF or CNS.

Administration can also be by transmucosal or transdermal means, in certain cases. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or vaginal suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery. Suitable formulations for transdermal and transmucosal administration include solutions, suspensions, gels, lotions and creams as well as discrete units such as suppositories and microencapsulated suspensions. Other delivery systems can include sustained release delivery systems which can provide for slow release of the active component of the invention, including sustained release gels, creams, suppositories, or capsules. Many types of sustained release delivery systems are available. These include, but are not limited to: (a) erosional systems in which the active component is contained within a matrix, and (b) diffusional systems in which the active component permeates at a controlled rate through a polymer. In another embodiment, pharmaceutical compositions may be delivered by ocularly via eyedrops.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, cherry, grape or orange flavoring.

The siRNA of the invention can be incorporated into pharmaceutical or antimicrobial compositions suitable for administration. Such compositions typically comprise the siRNA targeting an NLRC5 gene, and a pharmaceutically acceptable carrier as defined herein. Supplementary active compounds can also be incorporated into the compositions.

A pharmaceutical composition of the invention is formulated to be compatible with its intended route of administration. Generally, the compositions of the instant invention are introduced by any standard means, with or without stabilizers, buffers, and the like, to form a pharmaceutical composition. For use of a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the like.

In one embodiment, the invention features the use of the compounds of the invention in a composition comprising surface-modified liposomes containing poly (ethylene glycol) lipids (PEG-modified, or long-circulating liposomes or stealth liposomes). In another embodiment, the invention features the use of compounds of the invention covalently attached to polyethylene glycol. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). The long-circulating compositions enhance the pharmacokinetics and pharmacodynamics of therapeutic compounds, such as DNA and RNA, particularly compared to conventional cationic liposomes which are known to accumulate in tissues of the MPS (Liu et al., J. Biol. Chem. 1995, 42, 2486424870; Choi et al., International PCT Publication No. WO 96/10391; Ansell et al., International PCT Publication No. WO 96/10390; Holland et al., International PCT Publication No. WO 96/10392). Long-circulating compositions are also likely to protect drugs from nuclease degradation to a greater extent compared to cationic liposomes, based on their ability to avoid accumulation in metabolically aggressive MPS tissues such as the liver and spleen.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to hepatocytes) can also be used as pharmaceutically acceptable carriers. These can be prepared according to methods known to those skilled in the art, for example, as described in U.S. Pat. No. 4,522,811 U.S. Pat. No. 5,643,599, the entire contents of which are incorporated herein.

Liposomal suspensions (including liposomes targeted to macrophages containing, for example, phosphatidylserine) can also be used as pharmaceutically acceptable carriers. These can be prepared according to methods known to those skilled in the art, for example, as described in U.S. Pat. No. 4,522,811 U.S. Pat. No. 5,643,599, the entire contents of which are incorporated herein. Alternatively, the therapeutic agents of the invention may be prepared by adding a poly-G tail to one or more ends of the siRNA for uptake into target cells. Moreover, siRNA may be fluoro-derivatized and delivered to the target cell as described by Capodici, et al. (2002) J. Immuno. 169(9):5196.

Sterile injectable solutions can be prepared by incorporating the siRNA in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, in one embodiment, methods of preparation are vacuum drying and freeze-drying which yields a powder of the active ingredient plus any additional desired ingredient from a previously sterile-filtered solution thereof.

The siRNAs of the invention can be inserted into vectors. These constructs can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the vector can include the siRNA vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies typically within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of an siRNA (i.e., an effective dosage) ranges from about 0.001 to 3000 mg/kg body weight, in one embodiment, about 0.01 to 2500 mg/kg body weight, in one embodiment, the amount is about 0.1 to 2000 mg/kg body weight, and in another embodiment, the amount is about 1 to 1000 mg/kg, 2 to 900 mg/kg, 3 to 800 mg/kg, 4 to 700 mg/kg, or 5 to 600 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an siRNA can include a single treatment or, in one embodiment, can include a series of treatments.

For example, a subject is treated with an siRNA in the range of between about 0.1 to 20 mg/kg body weight, one time per week for between about 1 to 10 weeks, in one embodiment, between 2 to 8 weeks, in another embodiment, between about 3 to 7 weeks, and yet in another embodiment, for about 4, 5, or 6 weeks. It will also be appreciated that the effective dosage of siRNA used for treatment may increase or decrease over the course of a particular treatment. Changes in dosage may result and become apparent from the results of diagnostic assays as described herein.

It is understood that appropriate doses of the siRNAs or shRNAs, depend upon a number of factors within the skill of the ordinary physician, veterinarian, or researcher. The dose(s) of the agent will vary, for example, depending upon the identity, size, and condition of the subject or sample being treated, further depending upon the route by which the composition is to be administered, if applicable, and the effect which the practitioner desires the siRNA to have upon NLRC5.

The pharmaceutical compositions can be included in a container, pack, or dispenser together with instructions for administration, in certain cases.

### VII. Microbial Infections

In certain embodiments of the invention, an immune response is enhanced for a microbial infection. The enhancement may be preceded by the infection or subsequent to the infection. The microbial infection may be of any kind, but in particular embodiments it is a viral infection, fungal infection, or a bacterial infection. In specific embodiments, the microbe is pathogenic, causing disease or illness to its host.

Examples of viral microbes include those from Adenoviridae, Picornaviridae, Herpesviridae, Hepadnaviridae, Flaviviridae, Retroviridae, Orthomyxoviridae, Paramyxoviridae, Papovaviridae, Rhabdoviridae, or Togaviridae. Exemplary viral diseases include at least HIV, smallpox, influenza, mumps, measles, chickenpox, ebola, meningitis, and rubella.

The skilled artisan recognizes that many bacteria are harmless or even beneficial, although some pathogenic bacteria can cause infectious diseases. Examples of bacteria genera that can include pathogenic bacteria include Mycobacterium, Streptococcus, Pseudomonas, Campylobacter and Salmonella. Exemplary bacterial diseases include tuberculosis, pneumonia, foodborne illnesses, tetanus, typhoid fever, diphtheria, syphilis and leprosy.

Exemplary fungal infections include Tinea pedis, Candidiasis, Moniliasis, Aspergillus, Blastomycosis, Coccidioidomycosis, Cryptococcus, Fungal Sinusitis, Hypersensitivity Pneumonitis, Mucormycosis, Paracoccidioidomycosis, jock itch, and Sporotrichosis.

### VIII. Inflammation-Associated Disease

In certain aspects of the invention, an immune response is enhanced for an inflammation-associated disease. The skilled artisan recognizes that inflammation can result from pathogens, damaged cells, or irritants and can be either acute or chronic. The immune system is often associated with inflammatory disorders, for example in both allergic reactions and some myopathies; many immune system disorders result in abnormal inflammation. Non-immune diseases with a etiological origins in inflammatory processes are thought to include cancer, atherosclerosis, and ischaemic heart disease, for example. Examples of disorders associated with inflammation include at least the following: Asthma; Autoimmune diseases; Chronic inflammation; Chronic prostatitis; Glomerulonephritis; Hypersensitivities; Inflammatory bowel diseases; Pelvic inflammatory disease; Reperfusion injury; Rheumatoid arthritis; Transplant rejection; and Vasculitis.

### IX. Kits of the Invention

Any of the compositions described herein may be comprised in a kit. In a non-limiting example, an agent that inhibits NLRC5 is comprised in a kit. In specific embodiments, an additional agent, including an antimicrobial agent, an anti-inflammatory agent, and/or an anti-cancer agent may be comprised in a kit. The kits will thus comprise its contents in suitable container means.

The kits may comprise a suitably aliquoted agent that inhibits NLRC5, a pharmaceutical carrier, such as a lipid, and including a liposome, and, in some cases, an additional agent. Compositions of the present invention, whether labeled or unlabeled, as may be used to prepare a standard curve for a detection assay. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the NLRC5 inhibitor, lipid, additional agent, and any other reagent containers in close confinement for commercial sale. Such containers may include injection or blow molded plastic containers into which the desired vials are retained.

Therapeutic kits of the present invention are kits that may comprise an agent that inhibits NLRC5, such as siRNA molecules that are directed to NLRC5. Additional agents may include chemical compounds or pharmaceutically acceptable salts thereof, a protein, polypeptide, peptide, inhibitor, gene, polynucleotide, vector and/or other effector. Such kits may generally contain the compositions in a pharmaceutically acceptable formulation. The kit may have a single container means, and/or it may have distinct container means for each compound.

When the components of the kit are provided in one and/or more liquid solutions, the liquid solution is an aqueous solution, with a sterile aqueous solution being particularly preferred. The agent may also be formulated into a syringeable composition. In this case, the container means may itself be a syringe, pipette, and/or other such like apparatus, from which the formulation may be applied to an affected area of the body, injected into an animal, and/or even applied to and/or mixed with the other components of the kit. The formulation may be suitable for systemic or local delivery.

In some embodiments, the components of the kit may be provided as dried powder(s). When reagents and/or components are provided as a dry powder, the powder can be reconstituted by the addition of a suitable solvent. It is envisioned that the solvent may also be provided in another container means and may be sterile.

The container means will generally include at least one vial, test tube, flask, bottle, syringe and/or other container means, into which the inhibitory formulation is placed, and preferably is suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Irrespective of the number and/or type of containers, the kits of the invention may also comprise, and/or be packaged with, an instrument for assisting with the injection/administration and/or placement of the ultimate composition within or to the body of an animal. Such an instrument may be a syringe, pipette, forceps, and/or any such medically approved delivery vehicle.

In specific embodiments, the kit comprises an additional composition for treatment of a microbial infection (such as an antibiotic or antiviral agent), an inflammatory-associated disease (such as a steroid), or cancer (such as a chemotherapeutic, a hormonal drug, an immunotherapeutic, and so forth).

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Molecular Cloning and Characterization of NLRC5

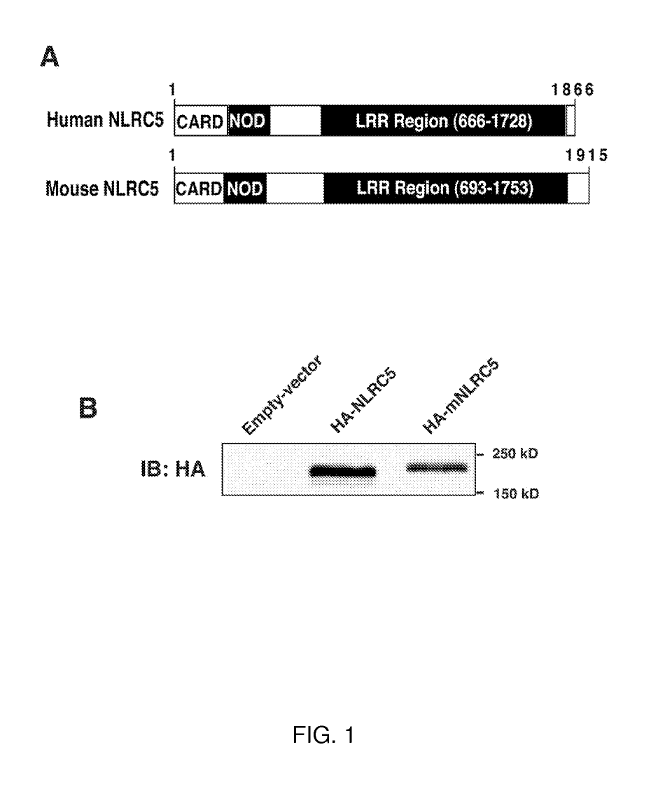

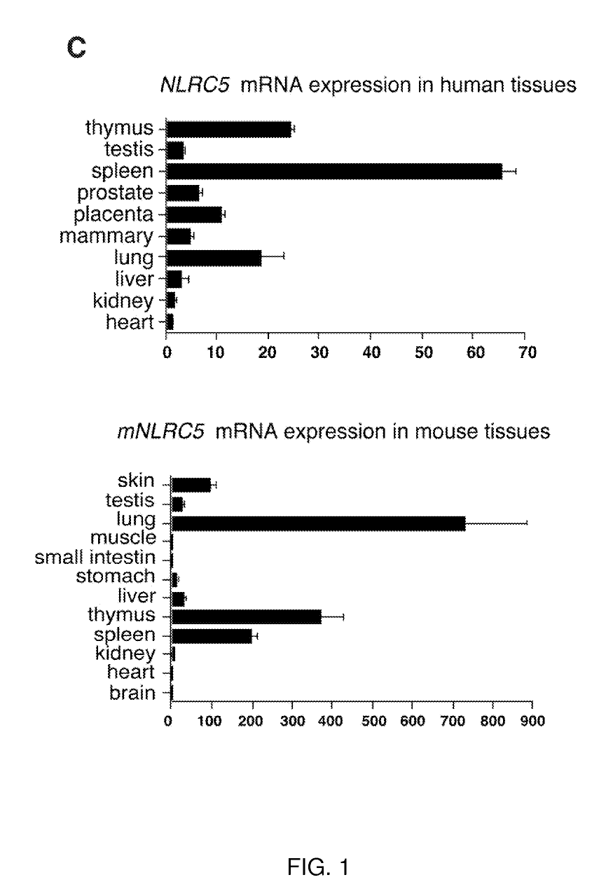

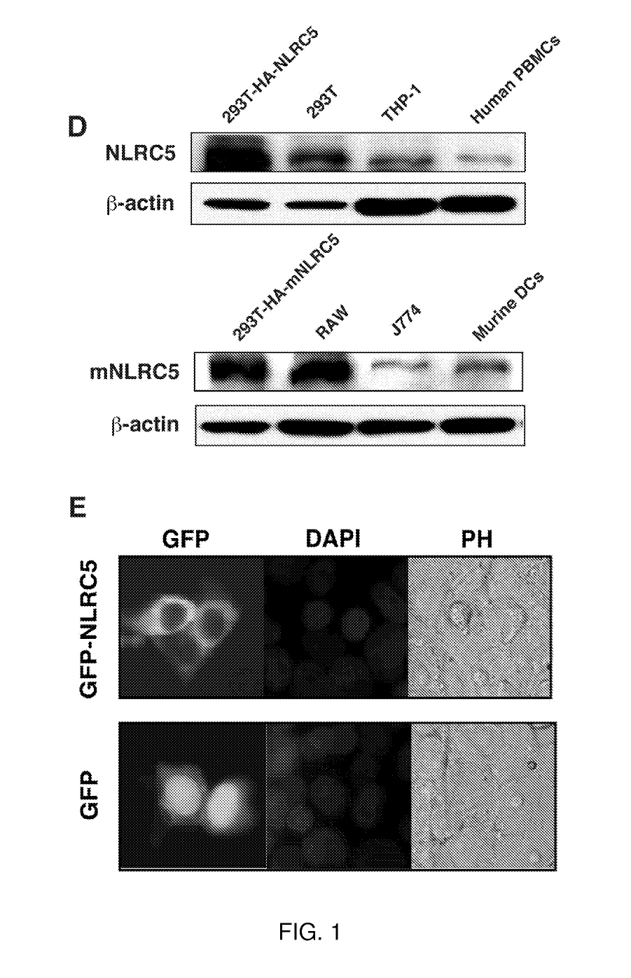

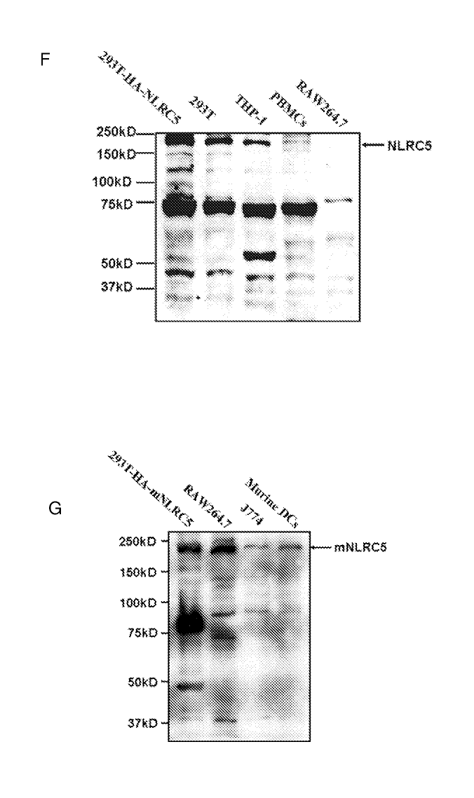

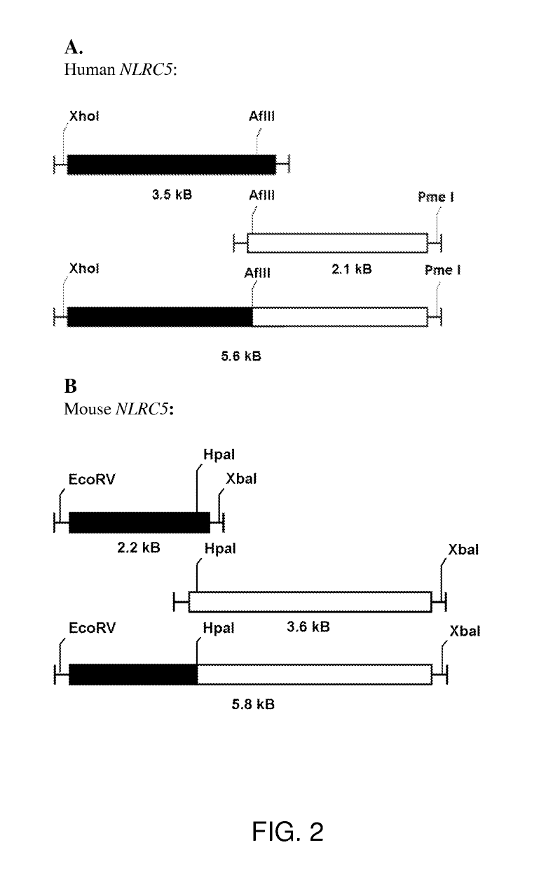

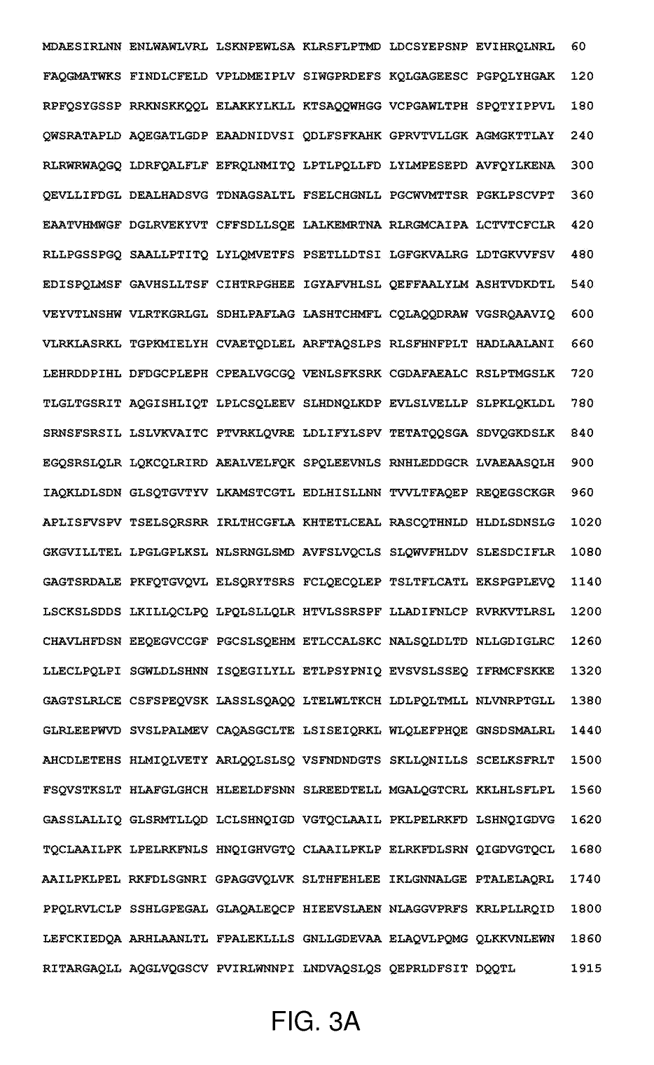

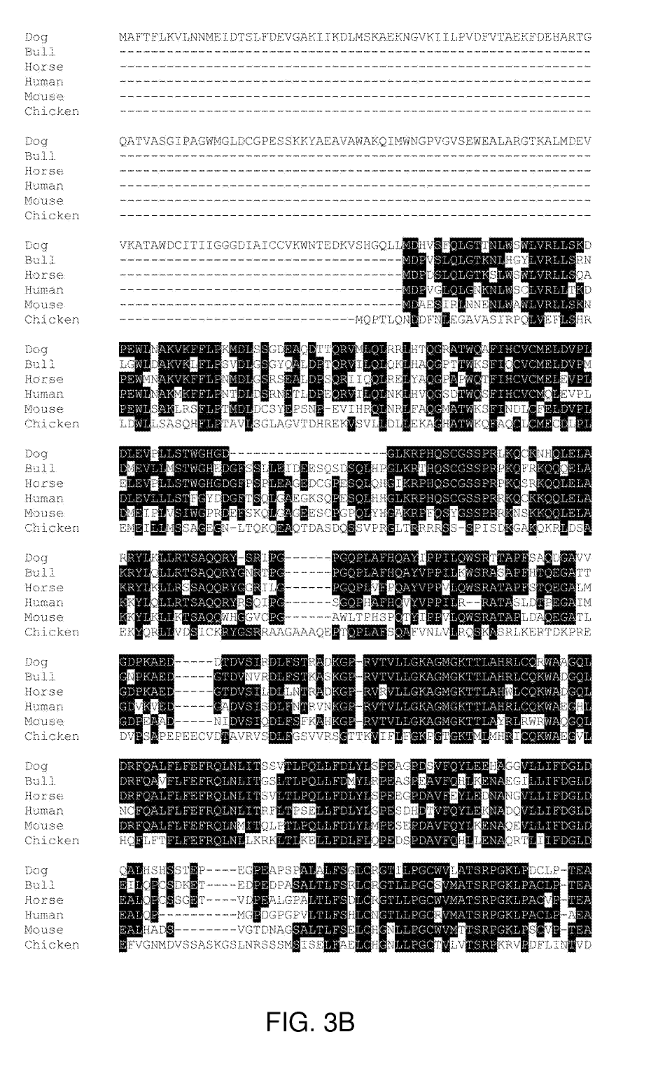

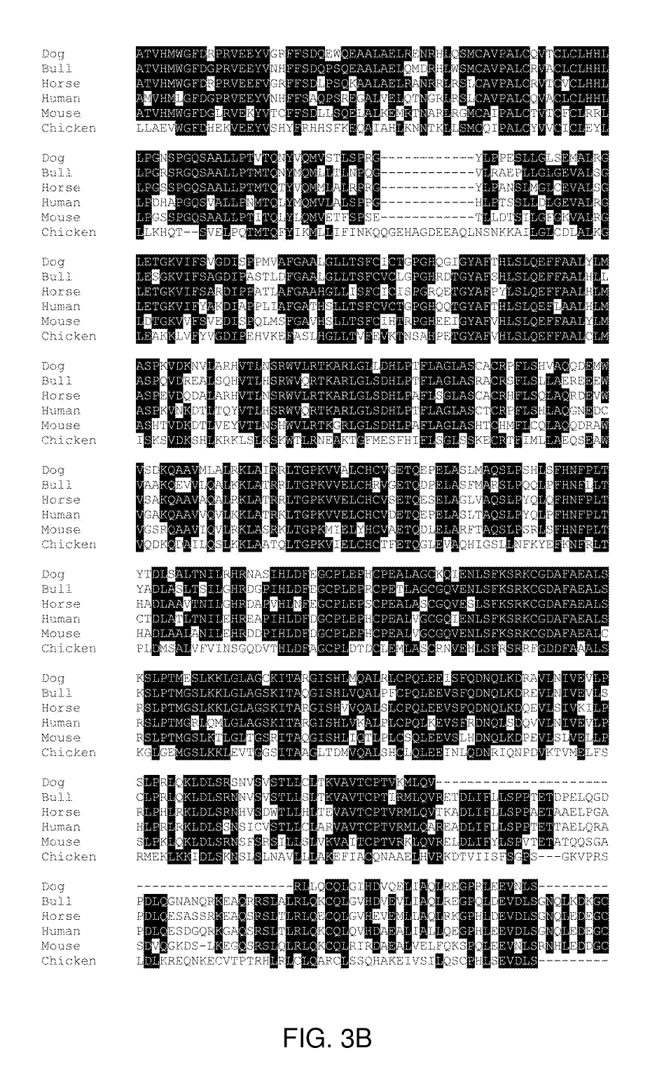

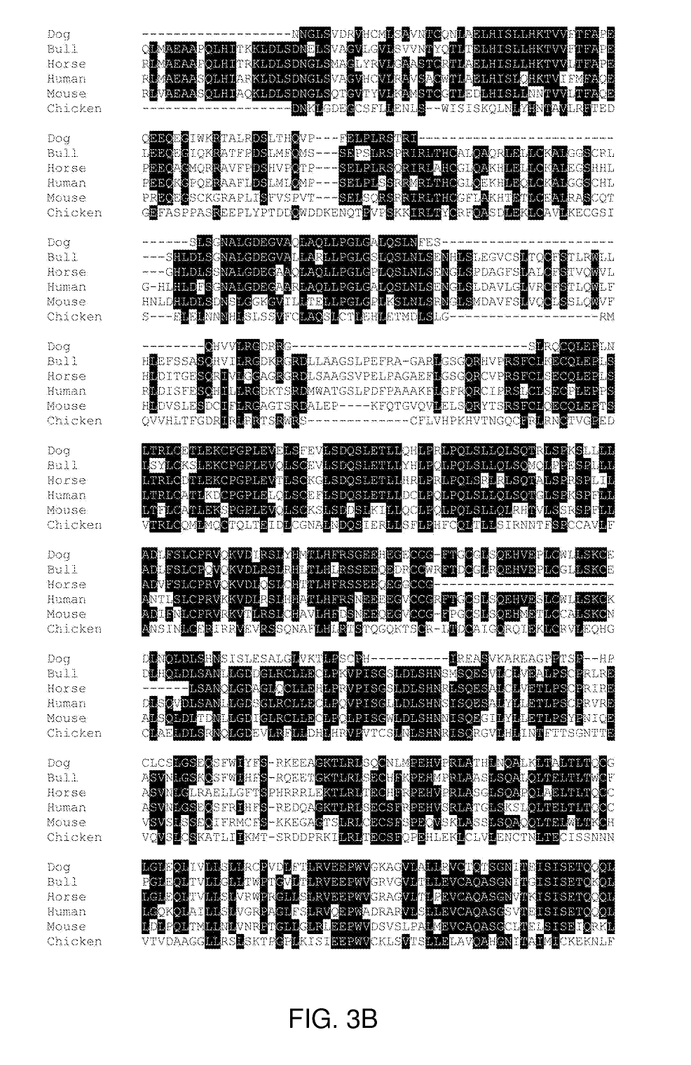

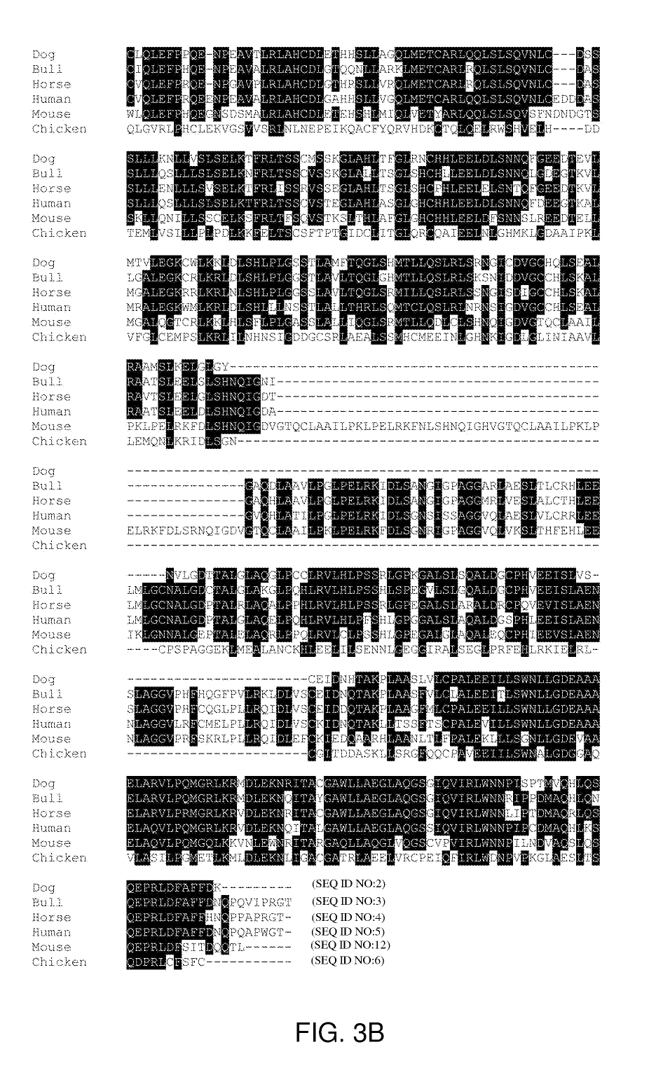

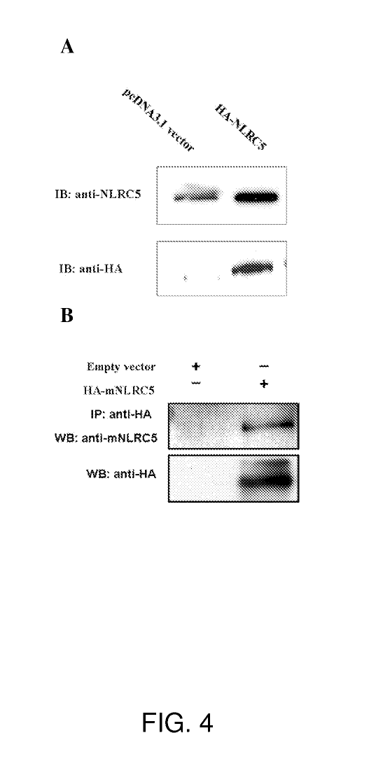

As a member of the NLR protein family, NLRC5 contains a CARD-like domain, a central NOD domain and a large LRR region (FIG. 1A), but its biological function remains unknown. Although database searches indicate that human NLRC5 encodes a protein of 1866 amino acids, the NLRC5 gene has not been cloned for functional studies, with the exception of a small DNA fragment encoding the N-terminal 90 amino acids (Dowds et al., 2003). To determine the function of NLRC5, a full-length (5.6 kb) human NLRC5 complementary DNA (cDNA) was cloned as well as a full-length (5.8 kb) murine NLRC5 (mNLRC5) by rapid amplification of cDNA ends (RACE) (FIG. 2). DNA sequence analysis revealed that the cloned human NLRC5 cDNA was identical to the one in the GenBank® database. By contrast, the cloned mNLRC5 cDNA encoded an ORF of 1915 amino acids and showed some differences in amino acid sequence compared with the sequences in the GenBank® databases (FIGS. 1A, 3A and 5B). There was a 64% amino acid sequence identity between the human and murine NLRC5 proteins. Expression of both HA-tagged NLRC5 and mNLRC5 was demonstrated by Western blotting with an anti-HA antibody. The apparent molecular weights of NLRC5 and mNLRC5 were about 205 and 215 kDa, respectively (FIG. 1B). NLRC5 homologs were identified in dog, bull, horse and chicken (FIG. 3C). Both human and murine NLRC5 mRNAs were strongly expressed in spleen, thymus, and lung, but weakly expressed in other tissues (FIG. 1C), indicating that this molecule is biologically conserved in these types of tissues, in certain aspect of the invention. To further demonstrate the protein expression level of NLRC5 in different cell types, polyclonal antibodies were generated against endogenous NLRC5 and mNLRC5, respectively (FIG. 4). Immunoblot analysis indicated that NLRC5 or its murine homolog was highly expressed in human HEK293T (293T) cells and mouse RAW264.7 cells (mouse macrophage cell line), but weakly expressed in human THP1 cells, peripheral blood mononuclear cells (PBMCs), mouse J774 cells and dendritic cells (FIG. 1D). To determine the cellular localization of NLRC5, 293T cells were transfected with NLRC5-GFP fusion DNA, and NLRC5-GFP was expressed in the cytoplasm, but not in the nucleus or mitochondria (FIG. 1E), indicating that NLRC5 is a cytosolic protein.

### Example 2

### NLRC5 is a potent negative regulator of NF-κB

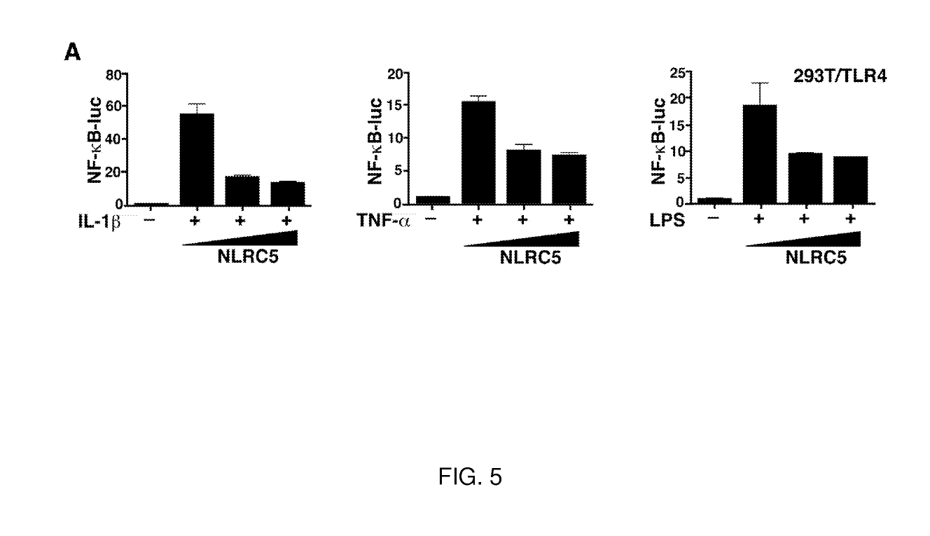

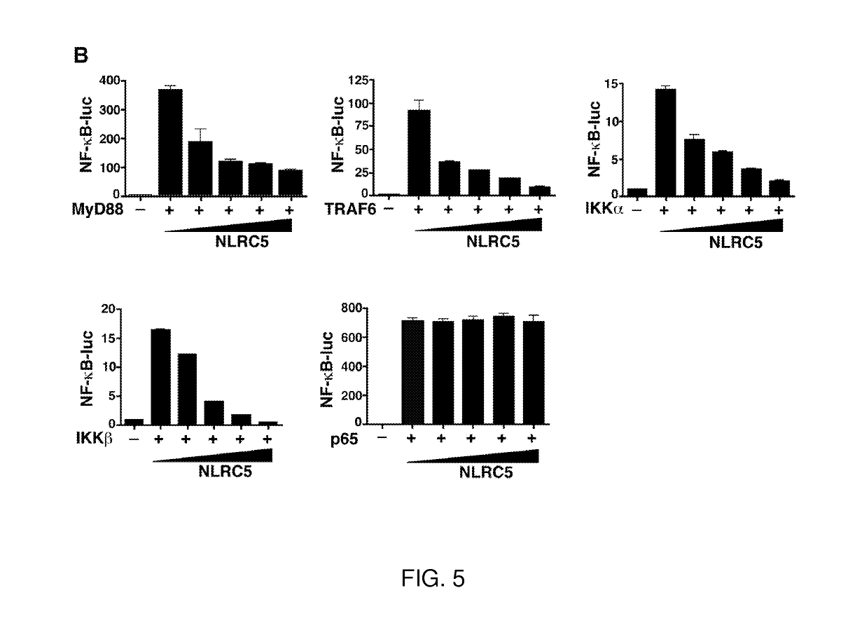

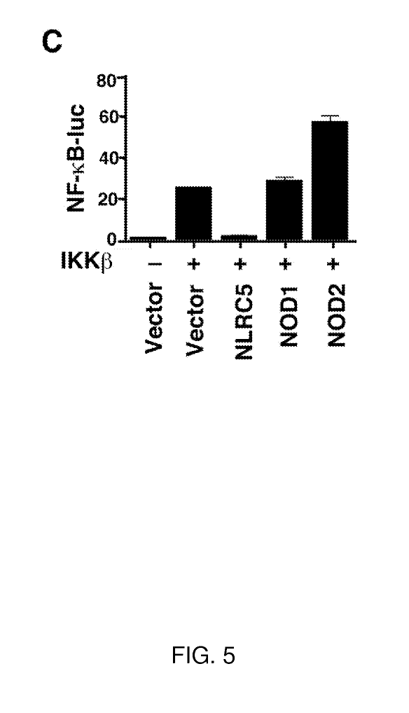

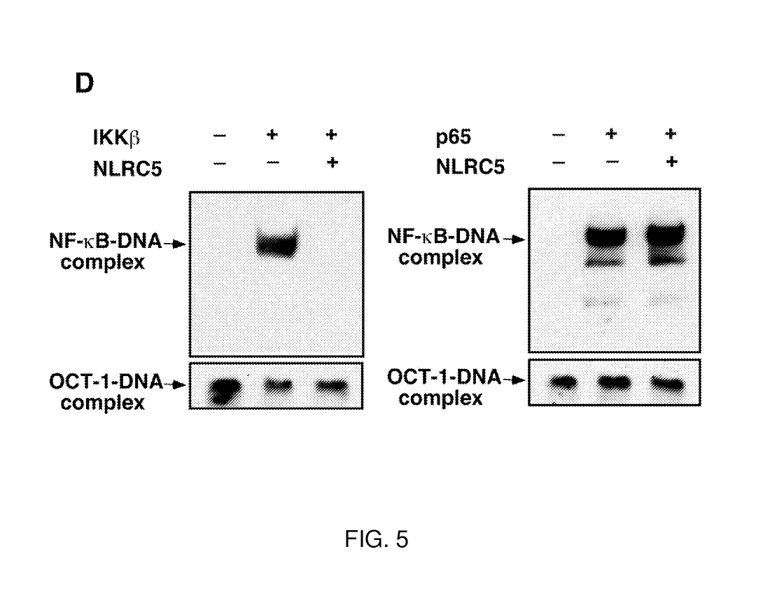

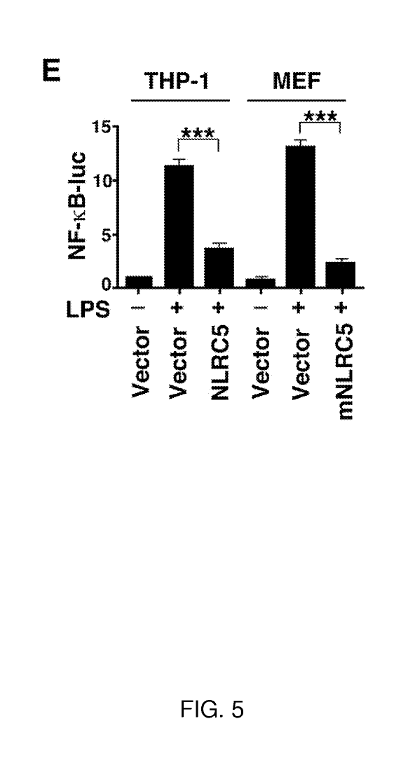

To determine whether NLRC5 is involved in TLR- and/or cytokine-mediated NF-κB activation, 293T or 293T/TLR4 cells were transfected with NF-κB-luc reporter DNA, with or without the NLRC5 plasmid, and then treated them with interleukin (IL)-1β, TNF-α or LPS. NF-κB-luciferase assays revealed that NLRC5 potently inhibited NF-κB activation induced by cytokines or LPS (FIG. 5A). Potential signaling molecules that activated the NF-κB-luc reporter were then sought. 293T cells were transfected with NF-κB-luc DNA together with MyD88, TRAF6, IKKα, IKKβ, or NF-κB (p65) plasmids. There was strong NF-κB activity in cells expressing MyD88, TRAF6, IKKα or IKKβ together with NF-κB-luc DNA, but such activity was inhibited when NLRC5 was cotransfected at increasing concentrations (FIG. 5B). By contrast, NLRC5 did not inhibit p65-mediated NF-κB-luc activity, indicating that in certain aspects of the invention it interacts with IKK signaling molecules upstream of p65 to block NF-κB activation (FIG. 1B). Neither control vector DNA nor NOD1 or NOD2 inhibited NF-κB activity (FIG. 5C). To determine whether the observed NLRC5-mediated inhibition of NF-κB-luc activity was associated with endogenous NF-κB activity, gel-mobility shift assays were performed. IKKβ, expression allowed endogenous NF-κB to bind to the biotin-HRP labeled DNA with NF-κB binding sites, but this activity was completely inhibited when NLRC5 was co-transfected. However, NLRC5 failed to inhibit p65-mediated NF-κB activation (FIG. 5D), consistent with results obtained with the NF-κB-luc reporter assay.

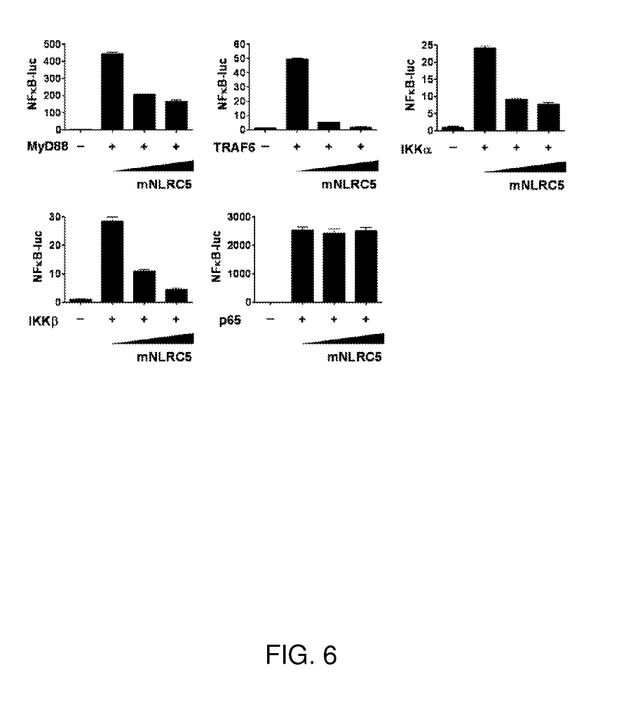

To substantiate these findings, it was tested whether the function of NLRC5 is conserved between humans and mice. Like its human homolog, mNLRC5 strongly inhibited NF-κB activation by MyD88, TRAF6, IKKα and IKKβ but not by p65 (FIG. 6). Moreover, it was tested whether NLRC5- or mNLRC5-mediated NF-κB inhibition in 293T cells could be extended to other cell types. Human THP-1 (monocytes) and murine embryonic fibroblasts (MEFs) were transfected with the NF-κB-luc reporter plasmid. LPS treatment of the transfected cells led to strong activation of NF-κB-luc, but this activity was markedly inhibited when either NLRC5 or mNLRC5 was cotransfected (FIG. 5E). Taken together, these results indicate that NLRC5 inhibits endogenous NF-κB activation and thus functions as a negative regulator of NF-κB activation induced by TNF-α, IL-1β, LPS or by their downstream signaling molecules, and that its biological function is conserved between humans and mice as well as among multiple cell types.

### Example 3

### NLRC5 Interacts with IKKα and IKKβ to Inhibit Their Phosphorylation

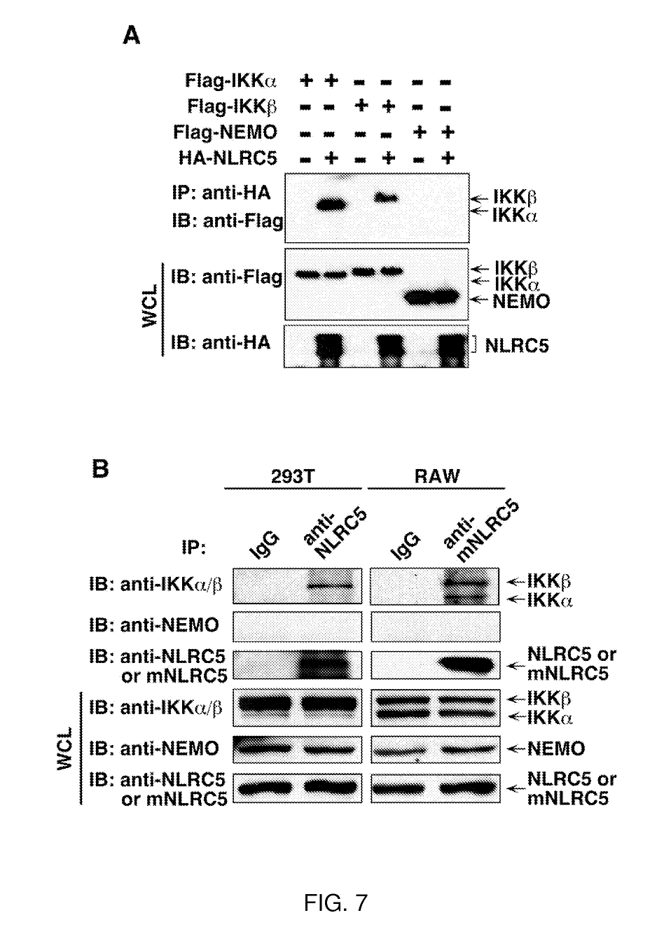

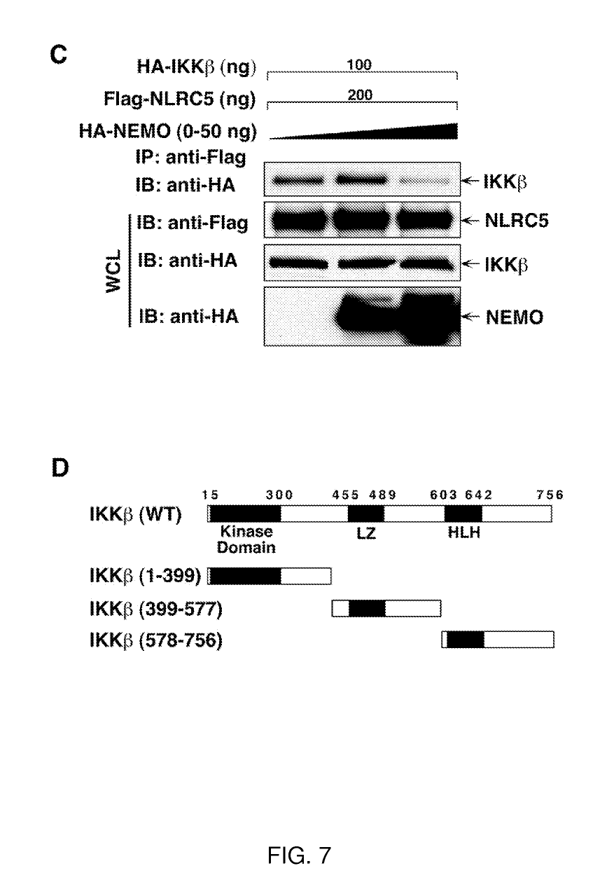

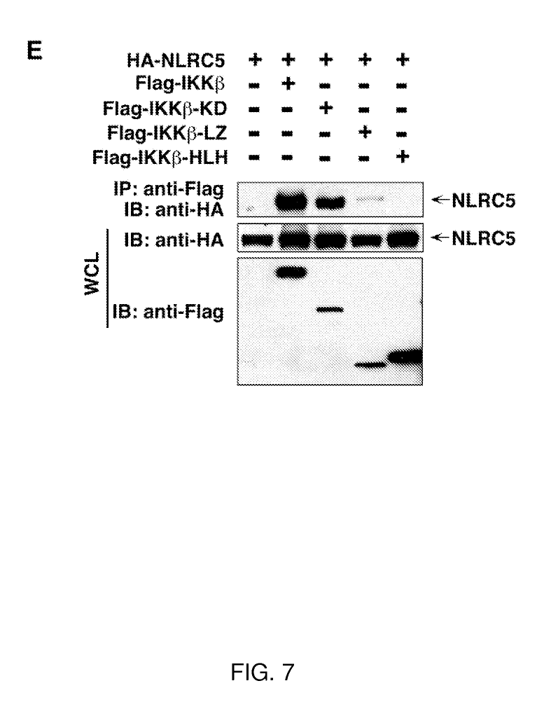

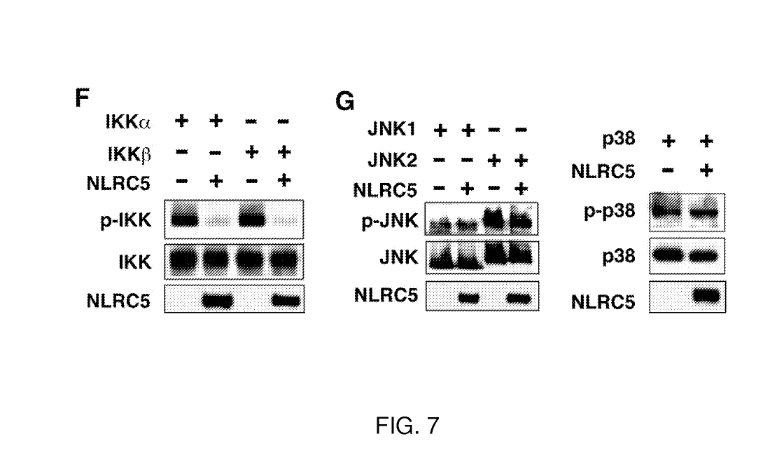

Results presented in FIGS. 5B and 5D indicate that NLRC5 directly interact with IKKα, IKKIβ or NEMO to inhibit NF-kB activation, in certain aspects of the invention. To characterize this, 293T cells were transfected with HA-tagged NLRC5 together with Flag-tagged IKKα, Flag-tagged IKKIβ or Flag-tagged NEMO expression plasmids. Communoprecipitation and Western blot analyses revealed that NLRC5 interacted with IKKα and IKKβ but not with NEMO, although the corresponding proteins were readily detected in whole cell lysates (FIG. 7A), indicating a specific interaction between NLRC5 and the IKKα/β complex.

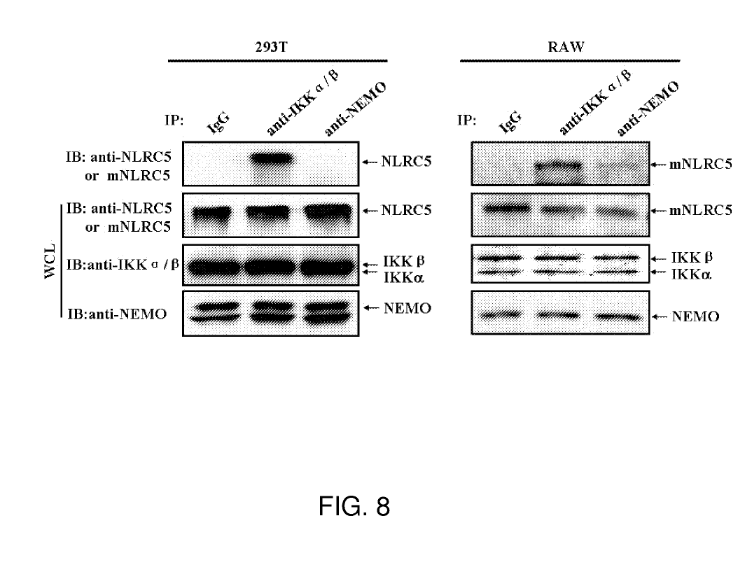

To further demonstrate that NLRC5 can interact with endogenous IKKα and IKKβ, 293T cell lysates were immunoprecipitated with an isotype IgG or anti-NLRC5 antibody, followed by Western blot analysis with the anti-IKKα/β, anti-NEMO or anti-NLRC5 antibody. As shown in FIG. 7B, the IKKα/β but not NEMO could be detected in the NLRC5-immunoprecipitated materials, even though all proteins were readily detected in the whole cell lysates. Similar studies were performed in mouse RAW264.7 cells, using an anti-mNLRC5 antibody for immunoprecipitation: IKKα/β was clearly apparent in the anti-mNLRC5 immunoprecipitates, but no NEMO signal was detected (FIG. 7B). A similar interaction between NLRC5 and IKK complexes was also observed when immunoprecipitation experiments were performed with a control IgG, anti-IKKα/β and anti-NEMO antibodies (FIG. 8). Taken together, these results indicate that NLRC5 can interact with IKKα and IKKβ, but not with NEMO, under physiological conditions. Since IKKα/β generally forms a complex with NEMO, it was next tested whether NLRC5 can compete with NEMO for binding to IKKα/β. 293T cells were transfected with a fixed concentration of Flag-NLRC5 and HA-IKKβ DNA together with increasing concentrations of HA-NEMO DNA. Cell lysates were coimmunoprecipitated by anti-Flag beads and immunoblotted by anti-HA antibody. The binding between NLRC5 and IKKβ was markedly decreased with increasing concentrations of NEMO (FIG. 7C). Collectively, these results indicate that NLRC5 competes with NEMO for binding to IKKα/β, in certain aspects of the invention.

Studies were then performed to determine which domain of IKKI3 interacts with NLRC5. Because IKKI3 contains an amino-terminal kinase domain (KD), a leucine zipper domain (LZ) and a carboxyterminal helix-loop-helix (HLH) domain, 3 deletion mutants of IKKβ were generated and tested for their ability to interact with NLRC5 in an immunoprecipitation assay. (FIG. 7D) Like the full-length IKKβ, the IKKβ-KD domain construct strongly interacted with NLRC5. By contrast, neither the IKKβ-LZ nor the IKKβ-HLH construct showed appreciable binding activity with NLRC5 (FIG. 7E). These results indicate that NLRC5 specifically binds to the IKKβ-KD domain, an interaction that may inhibit the phosphorylation of IKK complexes. To test this possibility, 293T cells were transfected with IKKα, NLRC5 or control vector, and the phosphorylation of IKK was measured. As shown in FIG. 7F, NLRC5 markedly inhibited the phosphorylation of IKKα and IKKβ. By contrast, the phosphorylation of p38 and JNK was not inhibited when either p38 or JNK was transfected with NLRC5 (FIG. 7G), indicating that NLRC5 specifically inhibits the phosphorylation of IKKα/β, without affecting other kinases such as p38 and JNK.

### Example 4

### LRR Region is Responsible for NLRC5-Mediated Inhibition of IKK Phosphorylation

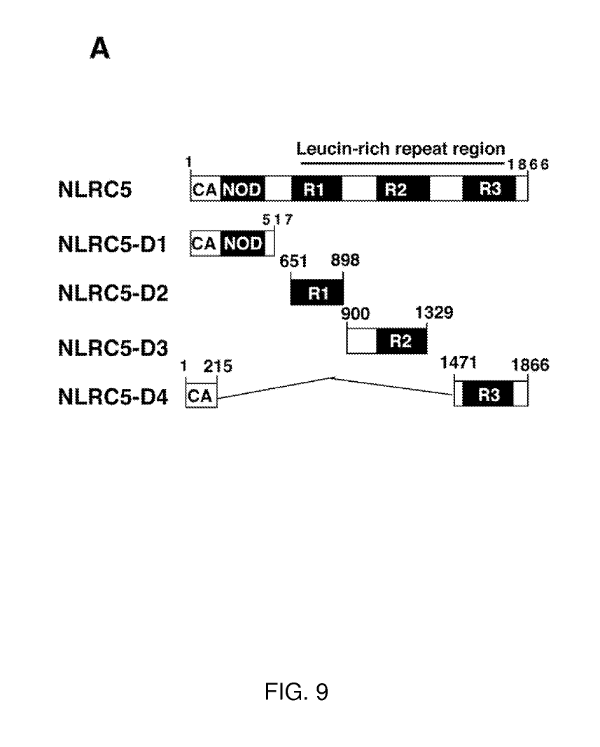

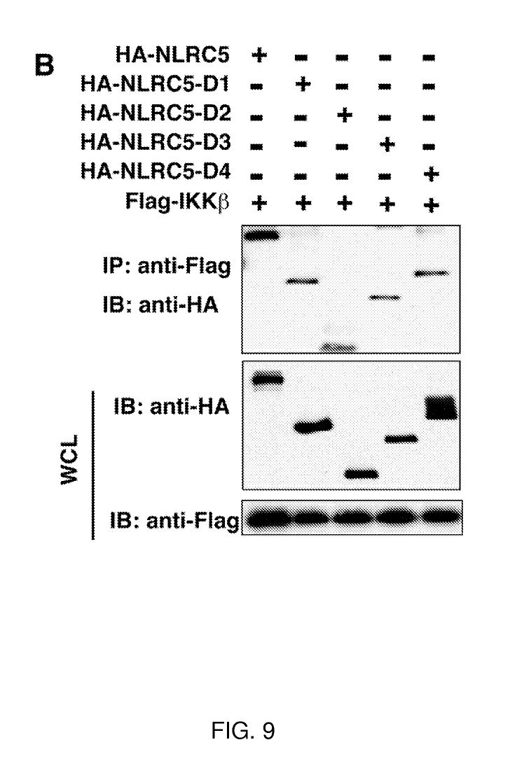

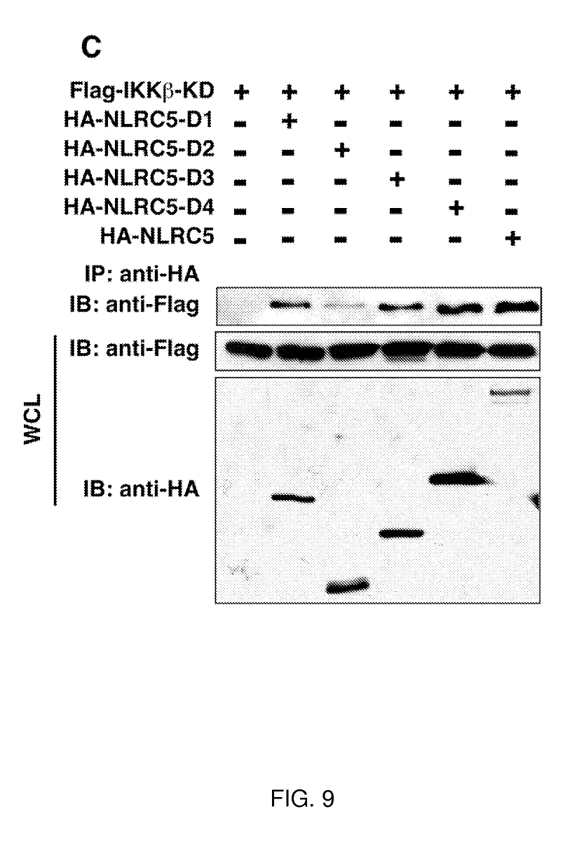

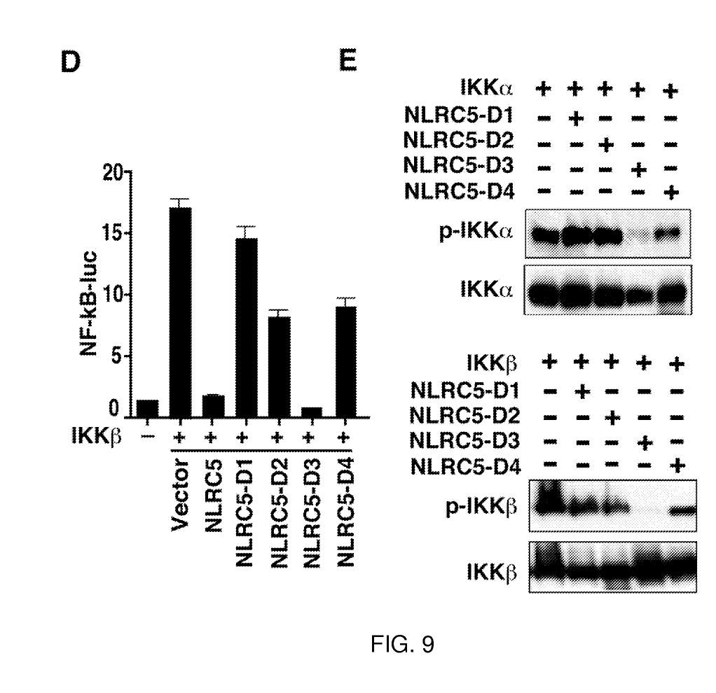

To identify the functional domains responsible for the observed inhibitory effects and interaction of NLRC5 with IKKα/β, four deletion constructs were generated: NLRC5-D1, containing the CARD and NOD domains (aa 1-517); NLRC5-D2, containing the LRR-R1 (aa 651-898); NLRC5-D3, containing the link region and LRR-R2 (aa 900-1329); and NLRC5-D4, containing the CARD domain and LRR-R3 (aa 1-215 plus 1471-1866) (FIG. 9A). Immunoprecipitation and western blot analyses revealed that all four NLRC5 deletion constructs could interact with the full-length IKKβ protein as well as IKKβ-KD (FIGS. 9B and 9C). These deletion constructs for their ability to inhibit NF-κB activation induced by IKKβ. Functional analysis showed that NLRC5-D1 did not inhibit NF-κB activation by IKKβ, while NLRC5-D2 and NLRC5-D4 partially inhibited NF-κB-luc activity (FIG. 9D). By contrast, NLRC5-D3, like the full-length NLRC5, could strongly inhibit NF-κB-luc activity. These results indicate that although all of the NLRC5 deletions can bind to IKKβ and its kinase domain, NLRC5-D3 is responsible for the observed inhibition of NF-κB activity by NLRC5.

To further investigate the molecular mechanism by which NLRC5-D3 inhibits NF-κB activity, it was tested whether these deletions can inhibit the phosphorylation of IKKα and IKKβ. NLRC5-D3 strongly inhibited IKKα and IKKβ phosphorylation, while NLRC5-D1, -D2 and -D4 produced little or only slight inhibition (FIG. 9E). Notably, the inhibitory activity of these NLRC5 deletion mutants on the phosphorylation of IKKα and IKKIβ correlated with their ability to inhibit NF-κB activation (FIG. 9D). Thus, in particular embodiments of the invention, NLRC5-D3 is critical for interacting with IKKα and IKKIβ as well as functionally inhibiting NF-κB activation by blocking the phosphorylation of IKK complexes, while NLRC5-D1, -D2 and -D4 can bind to IKKα and IKKβ molecules, but only weakly inhibit NF-κB activation.

### Example 5

### Knockdown of NLRC5 Enhances NF-κB Activation as Well as the Inflammatory Response

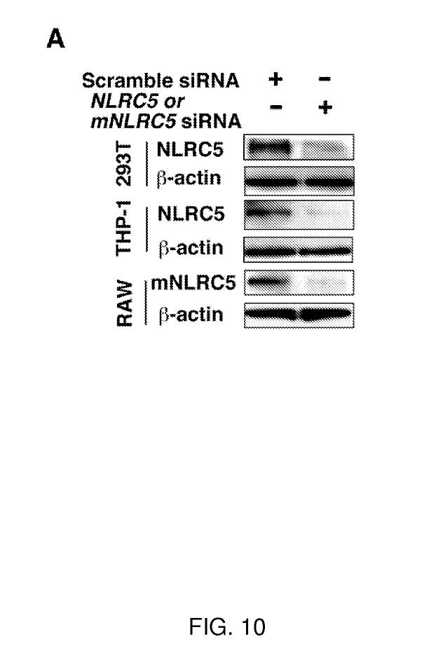

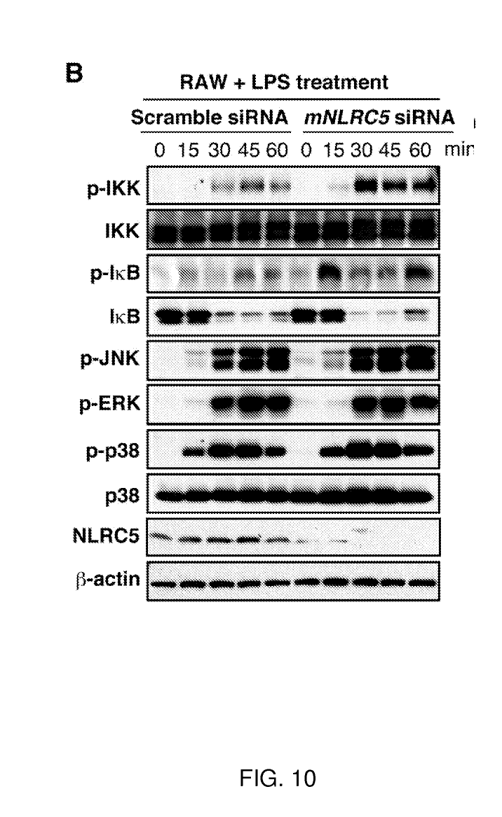

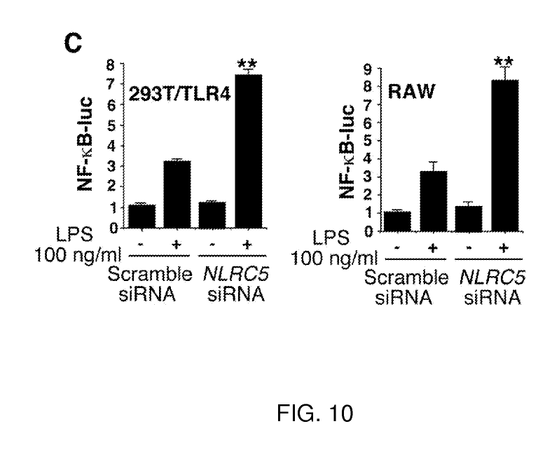

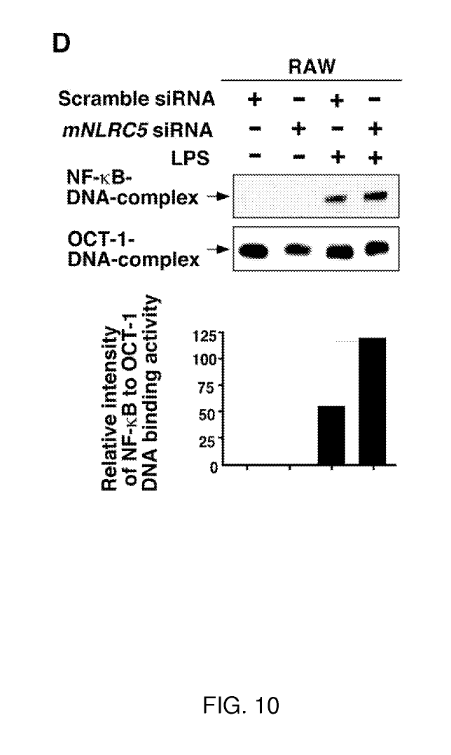

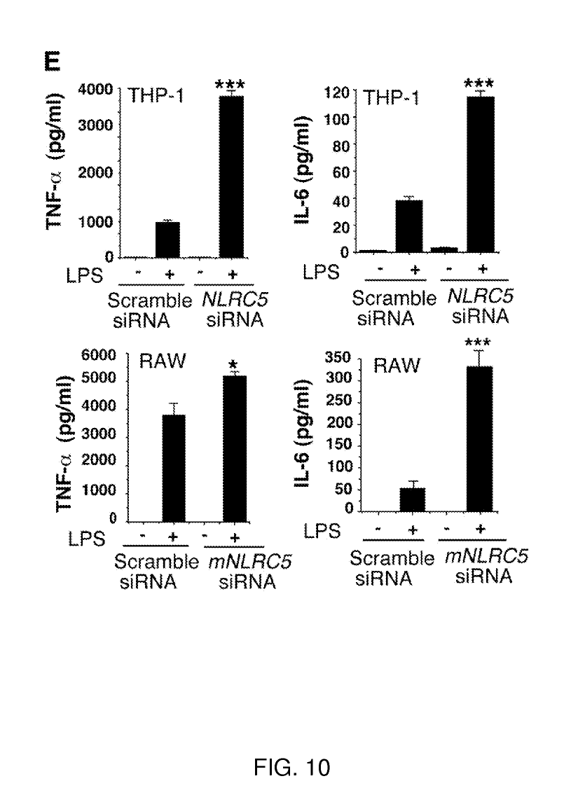

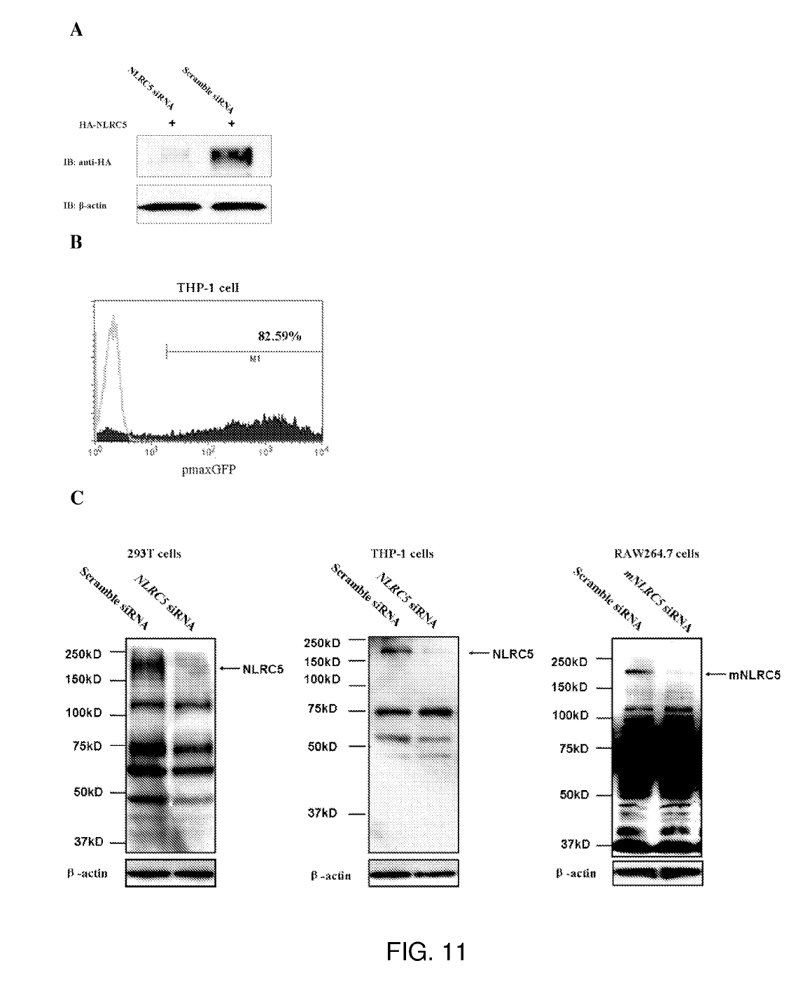

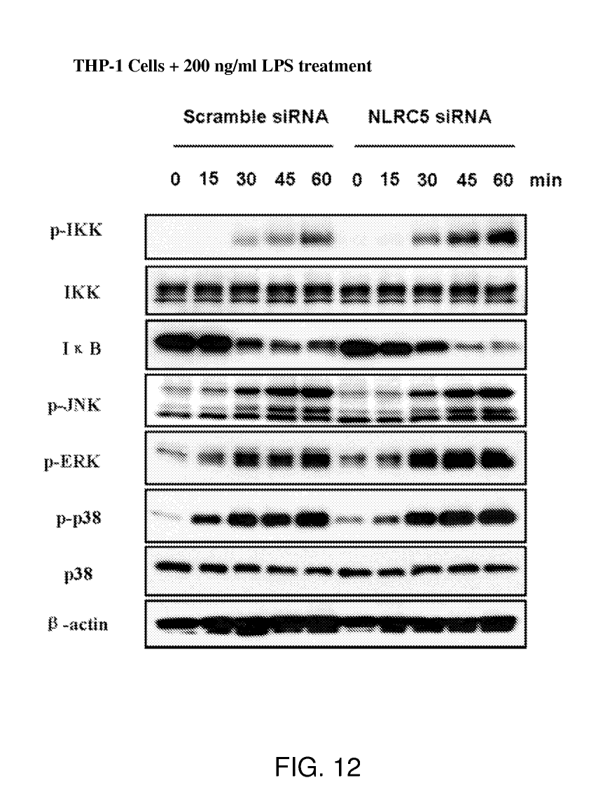

Since the endogenous and exogenous NLRC5 proteins specifically interact with IKKα/β and inhibit NF-κB activation, in certain aspects of the invention knockdown of NLRC5 would release IKKα and IKKIβ for increased NF-κB activation under physiological conditions. To characterize this, specific knockdown of NLRC5 in THP-1 cells and 293T cells or of mNLRC5 in RAW264.7 cells with their corresponding siRNAs, but not by scramble siRNAs, was demonstrated (FIG. 10A and FIG. 11). RAW264.7 cells were transfected with mNLRC5 or scramble siRNA and then treated them with LPS to evaluate the endogenous phosphorylation of IKK, IκB, and other kinases, including JNK, ERK and p38. As shown in FIG. 10B, the phosphorylation of IKK (p-IKK) and IκB (p-IκB) in the mNLRC5-siRNA transfected cells was at least 5-fold higher than that in the scramble siRNA-transfected cells at 15 and 30 min after LPS treatment, although the total amounts of IKK and IKB proteins were comparable between mNLRC5 siRNA- and scramble siRNA-transfected cells. More importantly, no appreciable differences were observed in p-JNK, p-ERK and p-p38 between scramble siRNA- and mNLRC5 siRNA-transfected cells, clearly indicating the specific inhibitory effect of mNLRC5 on the endogenous phosphorylation of IKK, but not on JNK, ERK and p38 phosphorylation. Similar results were obtained with human THP-1 cells (FIG. 12). Collectively, these results indicate that specific knockdown of NLRC5 or mNLRC5 enhances the phosphorylation of the IKK complexes, but does not affect the phosphorylation of JNK, ERK and p38.

It was next determined whether the enhanced endogenous phosphorylation of IKK seen with NLRC5 knockdown promotes NF-κB activation and NF-κB-dependent gene expression. Using the NF-κB-luc reporter assay, knockdown of endogenous NLRC5 by specific siRNA markedly increased NF-κB-luc activity in 293T/TLR4 and RAW264.7 cells after LPS treatment, compared with that in cells transfected with scramble siRNA (FIG. 10C). To directly demonstrate that specific knockdown of mNLRC5 enhances endogenous NF-κB activity, a gel-mobility shift assay was used to examine the endogenous NF-κB DNA binding activity in RAW264.7 cells transfected with mNLRC5-specific or scramble siRNAs. Results in FIG. 10D show that endogenous NF-κB activity in cells transfected with mNLRC5-specific siRNA was at least 2-fold higher than that in cells transfected with scrambled siRNA. Consistent with this observation, knockdown of NLRC5 or mNLRC5 resulted in markedly increased secretions of the pro-inflammatory cytokines encoded by NF-κB-responsive genes, such as TNFα and IL-6, in both THP-1 and RAW264.7 cells treated with LPS, compared with cells treated with scramble siRNA (FIG. 10E). Thus, in certain aspects of the invention, knockdown of NLRC5 or mNLRC5 not only enhances the phosphorylation of IKKα and IKKβ but also stimulates endogenous NF-κB activity, thus increasing NF-κB-dependent proinflammatory cytokine responses under normal physiological conditions.

### Example 6

### NLRC5 Negatively Regulates Type I Interferon-β Signaling by Interacting with RIG-I AND MDA5

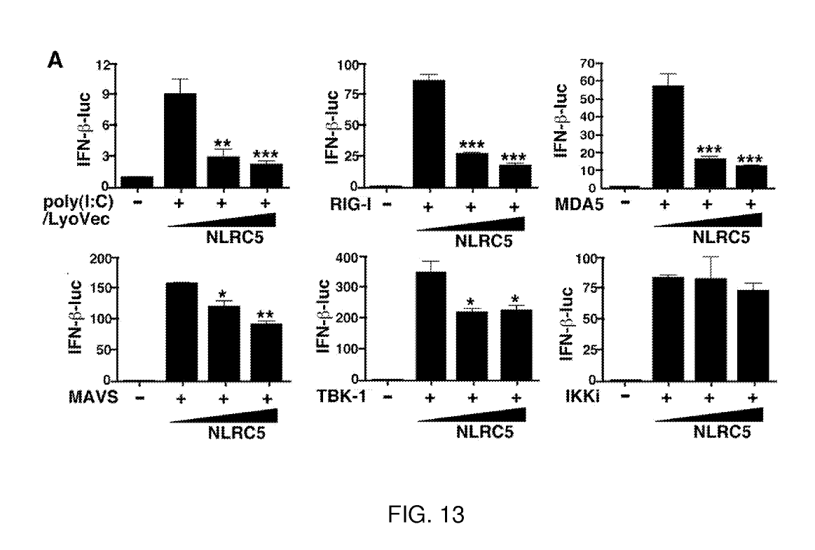

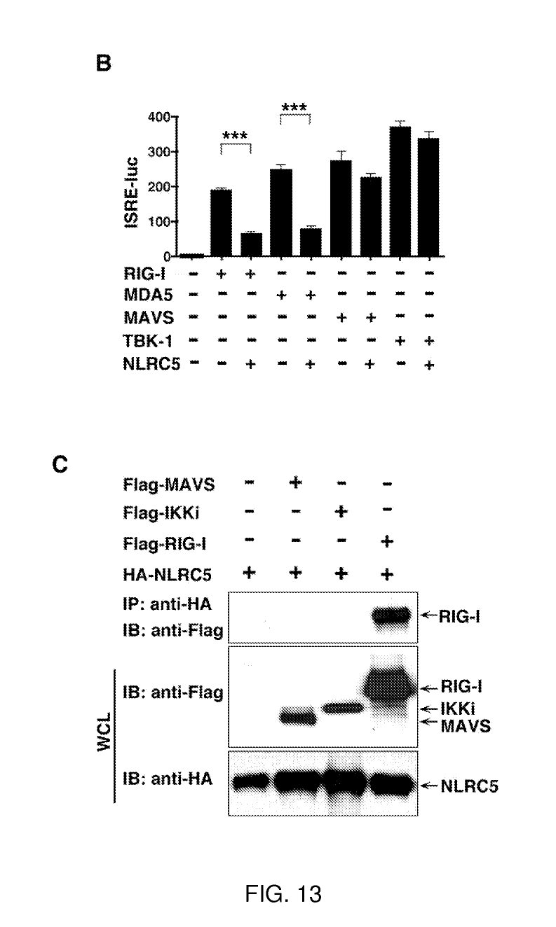

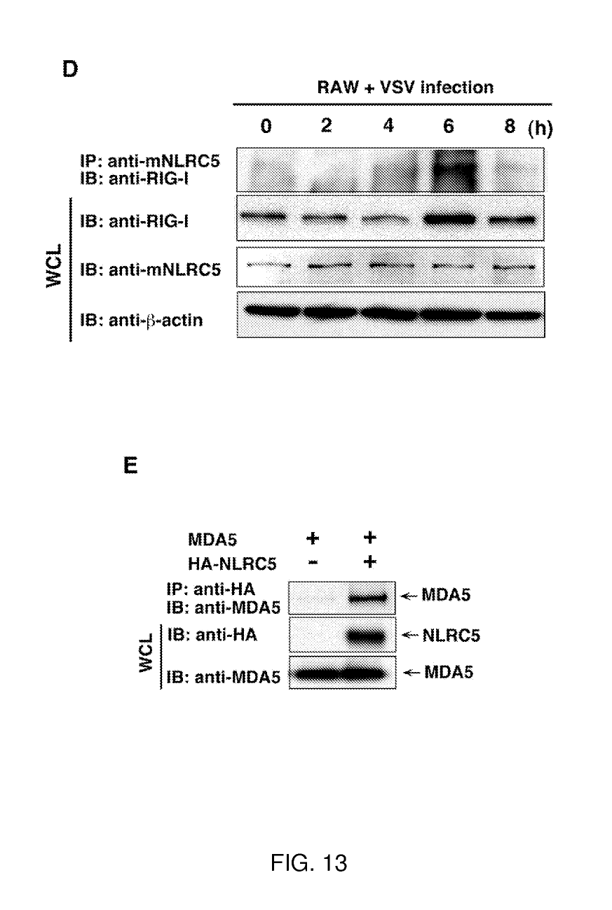

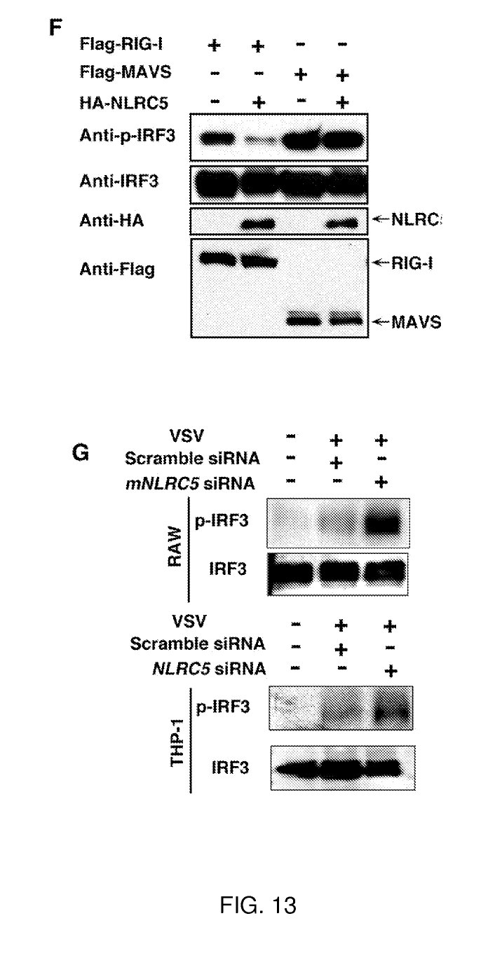

Recent studies show that NLRX1, a member of the NLR protein family, functions as a mitochondrial protein that interacts with the mitochondrial adaptor MAVS to inhibit the RIG-1-mediated signalling pathway and triggers the generation of reactive oxygen species (Moore et al., 2008; Tattoli et al., 2008). To determine whether NLRC5 might also be involved in the regulation of type I interferon signaling mediated by RLRs (RIG-1- and MDA-5), TLR3-deficient 293T cells were transfected with luciferase reporter DNA and intracellular poly(I:C) (a ligand for MDA5), with or without NLRC5, and the cells for IFN-β-dependent luciferase activity were then evaluated. As expected, intracellular poly(I:C) activated IFN-signaling by comparison with results in cells lacking this ligand; however, the poly(I:C)-induced IFN-β responses were strongly inhibited by NLRC5 (FIG. 13A), indicating that NLRC5 functions as a negative regulator of this antiviral pathway.

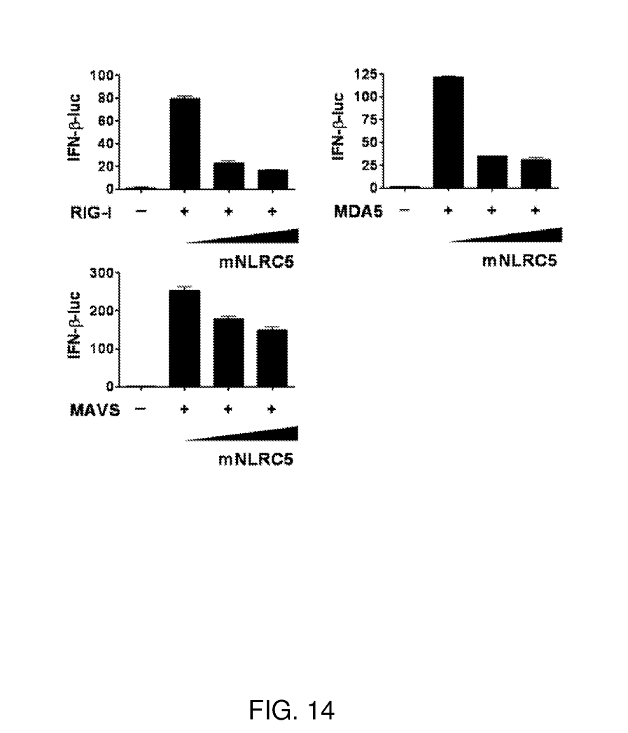

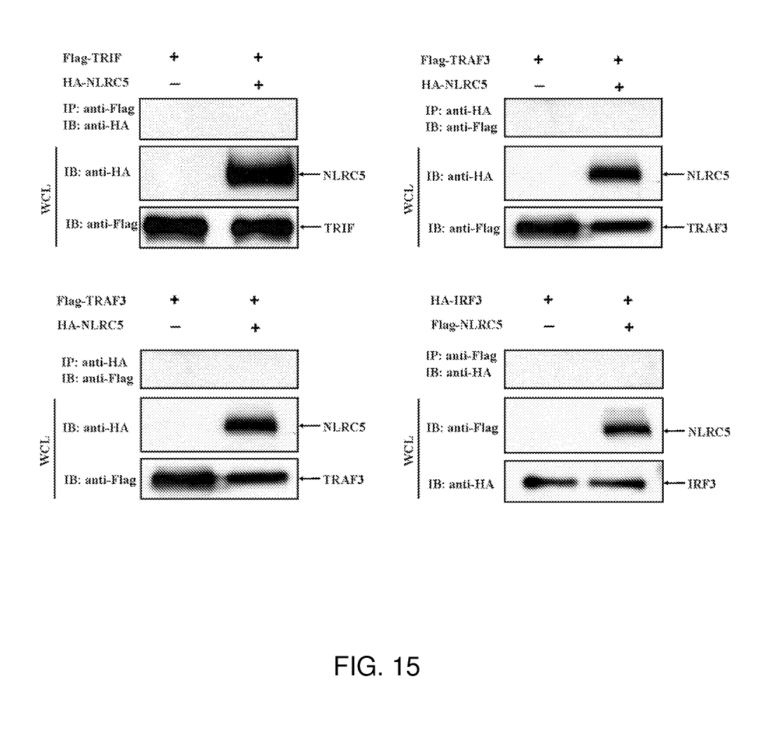

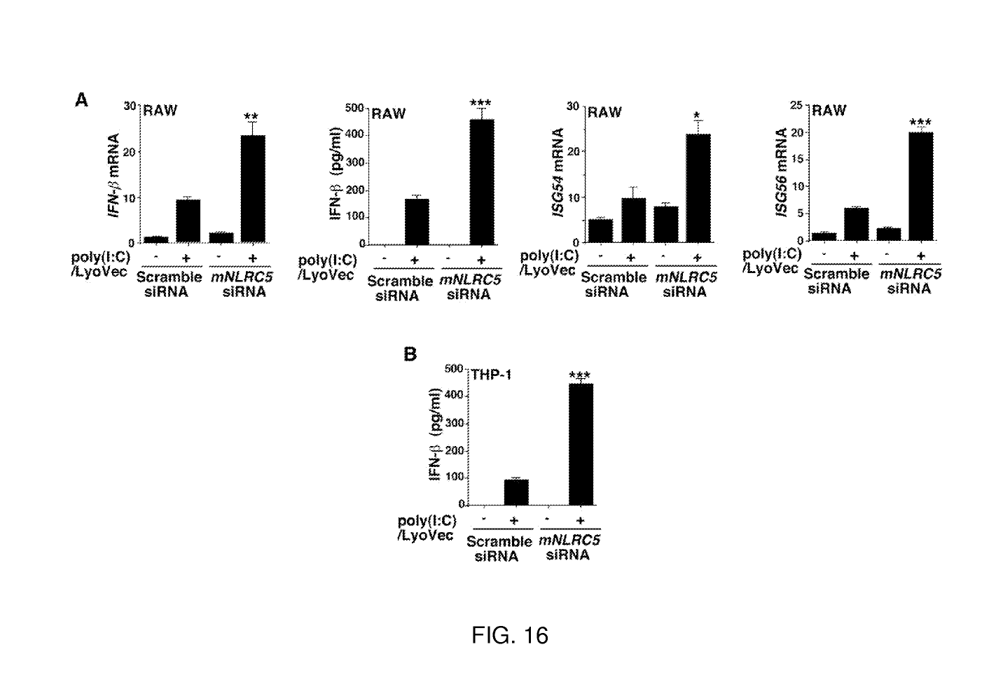

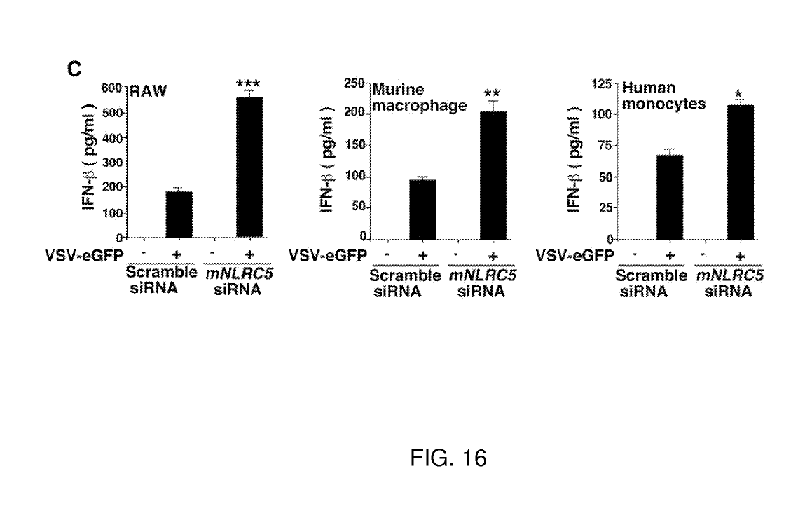

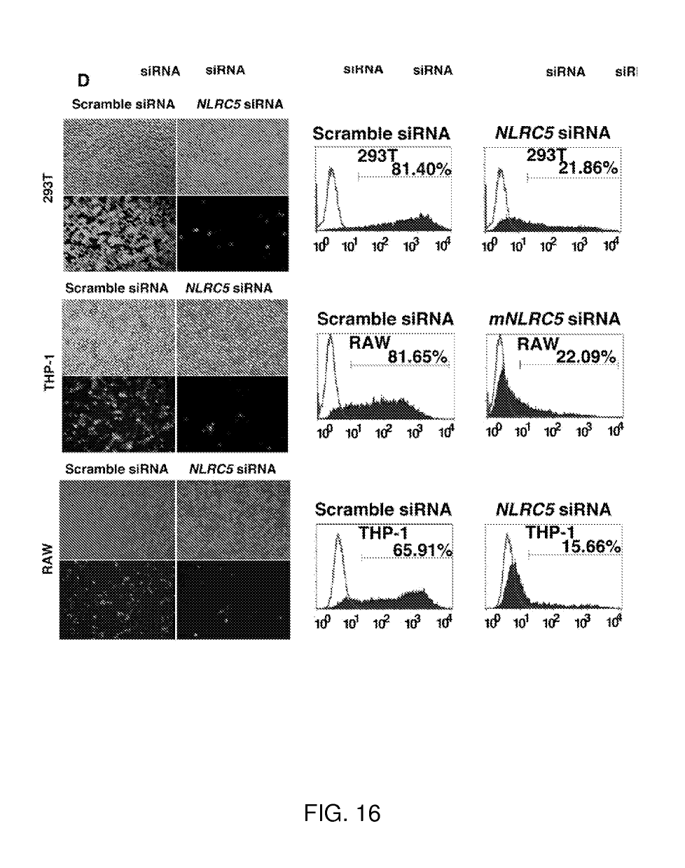

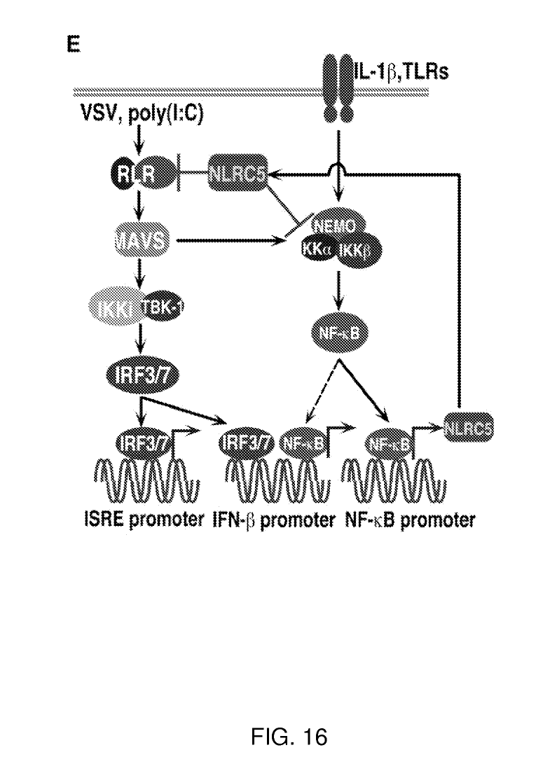

The molecular mechanisms by which NLRC5 could inhibit the IFN-β response was investigated. In 293T cells transfected with RIG-I, MDA5, MAVS, TBK1 and IKKi, each of which strongly activates the IFNβ response, both RIG-1- and MDA5-induced IFN-β-luc activities were markedly inhibited by increasing concentrations of NLRC5. By contrast, MAVS- and TBK1-induced IFN-β-luc activity was weakly inhibited, while IKKi-induced IFN-β luciferase activity remained unchanged (FIG. 13A). Similar results were obtained with mNLRC5 (FIG. 14). Weak inhibition of MAVS- and TBK1-induced IFN-β-luc activity by NLRC5 may be due to the inhibitory effect of NLRC5 on the IKK complexes, but not on any direct effect of MAVS or TBK1 itself, as optimal IFN-β promoter activity requires cooperation between IRF3 and NF-κB activation (Zhong et al., 2008)(FIG. 16E). To characterize this, 293T cells were transfected with ISRE-luc (which could be activated by IRF3), together with RIG-I, MDA5, MAVS or TBK1 in the presence or absence of NLRC5. Both RIG-I- and MDA5-induced ISRE-luc activity were potently inhibited, while MAVS and TBK1-induced ISRE-luc activity were not (FIG. 13B). These results clearly indicate that NLRC5 directly inhibits RIG-I and MDA5, but not MAVS or TBK1. To confirm this finding, immunoprecipitation studies were performed, and NLRC5 was strongly associated with RIG-I and MDA5, but did not interact with MAVS, IKKi, TBK1, TRIF, TRAF3, or IRF3 (FIG. 13C, 13E and FIG. 15). Thus, NLRC5 specifically binds to the RIG-I and MDA5 proteins to inhibit the IFN-β response.

To further characterize the interaction between RIG-I and NLRC5 under physiological conditions, RAW264.7 cells were infected with vesticular stomatitis viruses expressing enhanced green fluorescence protein (VSV-eGFP), which is known to activate the RIG-I signalling pathway, and immunoprecipitation was performed with anti-mNLRC5 at different time points after VSV-eGFP infection. Immunoblot analysis with anti-RIG-I antibody failed to detect RIG-I protein (or only slight faint band) in immunoprecipitated materials over the first 0, 2 and 4 h of infection, although both NLRC5 and RIG-I were readily found in the whole cell lysates (FIG. 13D). By contrast, a strong RIG-I protein band was observed in the immunoprecipitated materials collected at 6 h postinfection, which became weak at 8 h postinfection. Notably, RIG-I protein expression was increased at 6 h postinfection, consistent with the observation that RIG-I expression can be induced by VSV infection (Honda and Taniguchi, 2006). These results indicate that NLRC5 binds to RIG-I after VSV-eGFP infection, in particular aspects of the invention.

To explain the ability of NLRC5 to inhibit the type I interferon pathway, it was considered that it prevents the phosphorylation of IRF3, a transcription factor critical for the type I interferon response. Thus, in certain aspects of the invention, overexpression of NLRC5 would inhibit IRF3 phosphorylation, while its knockdown would enhance IRF3 phosphorylation as well as the expression of IFN-responsive genes. Indeed, NLRC5 overexpression potently blocked the phosphorylation of endogenous IRF3 induced by RIG-I, but not by MAVS (FIG. 13F). Conversely, siRNA knockdown of NLRC5 or mNLRC5 in RAW264.7 and THP-1 cells markedly increased the phosphorylation of IRF3, compared with the result in scramble siRNA-transfected cells (FIG. 13G). Thus, NLRC5 can downregulate IFN-β signalling by inhibiting IRF3 phosphorylation via interaction with RLRs.

### Example 7

### Knockdown of NLRC5 Enhances Antiviral Immunity

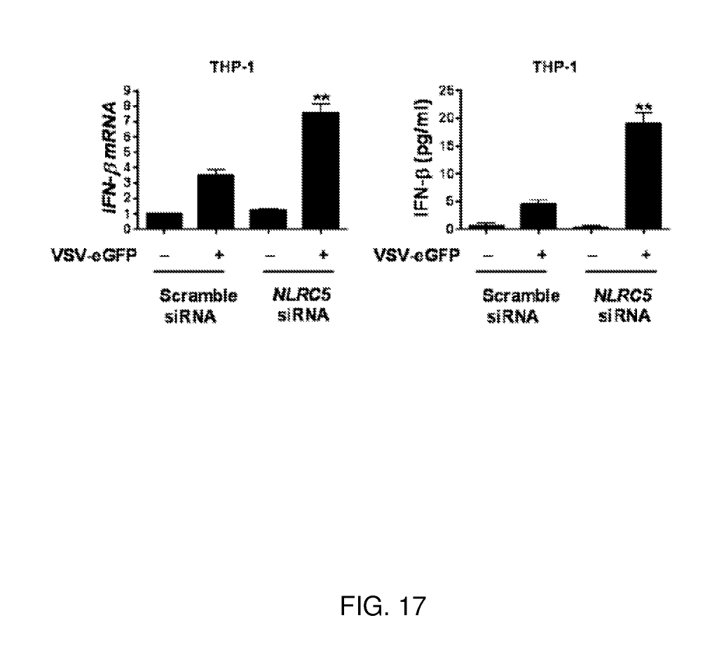

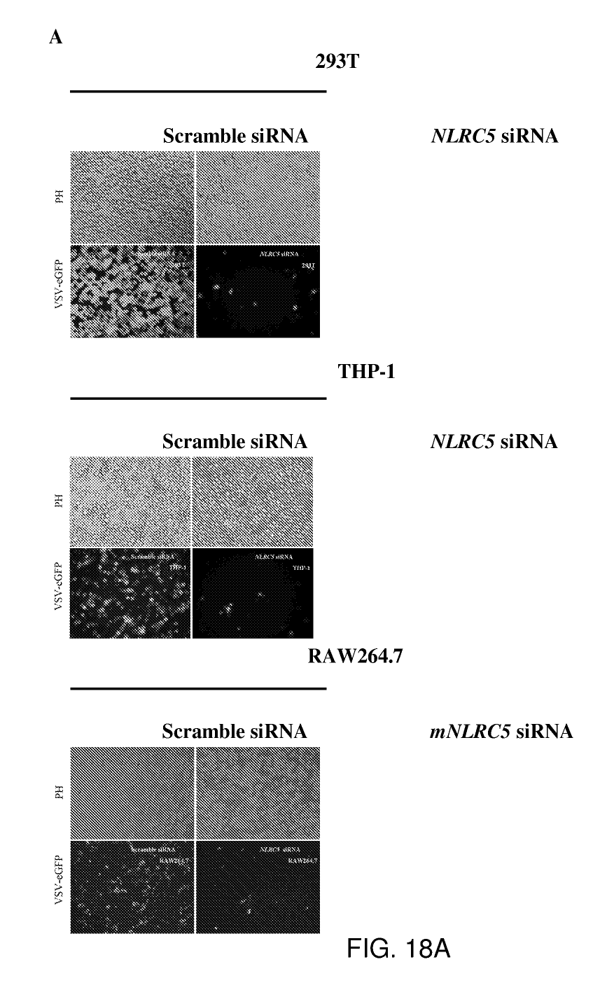

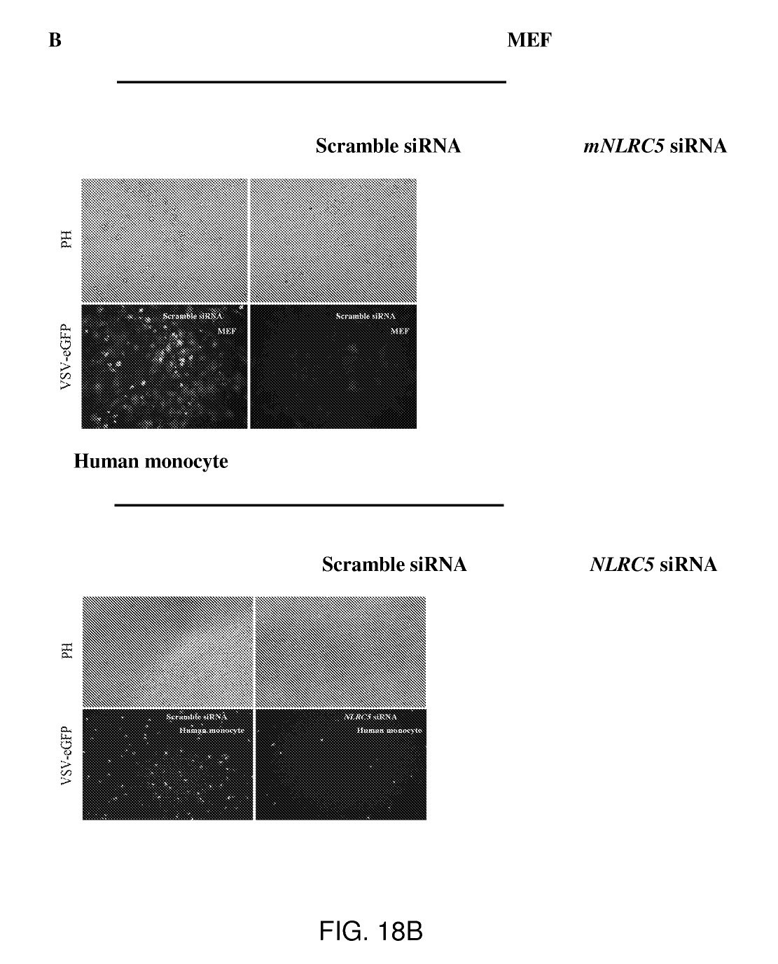

To further demonstrate the effects of NLRC5 knockdown on expression of IFN-responsive genes, endogenous mNLRC5 was knocked down in RAW264.7 or THP-1 cells with mNLRC5 or NLRC5 specific siRNA and then the cells were treated with poly(I:C)/Lyovec (a ligand for MDA5) or infect them with VSV-eGFP. Real-time PCR analysis revealed that poly(I:C)/Lyovec treatment strongly increases mRNA levels of IFN-β, and the interferon-stimulating genes ISG54 and ISG56 as well as IFN-β protein production in cells transfected with mNLRC5-specific siRNA (FIGS. 16A and 16B). These findings are consistent with a previous study showing that the mRNA level of IFN-β is upregulated when NLRC5 is knocked down in A549 cells (Opitz et al., 2006). More importantly, VSV-eGFP infection also led to a large increase in the production of IFN-β protein in RAW264.7 cells, primary murine macrophages and human monocytes transfected with mNLRC5 or NLRC5-specific siRNA, compared to cells transfected with scramble siRNA (FIG. 16C), although the secretion of IFN-β in THP-1 cells is quite low compared to that in other cell types (FIG. 17). In addition siRNA-mediated specific knockdown rendered cells remarkably resistant to viral infection and reduced the levels of VSV-eGFP-positive cells among 293T, THP-1, and RAW264.7 cells, as well as MEFs and human monocytes, compared with the same cells transfected with scramble siRNAs (FIG. 16D and FIG. 18). Knockdown of endogenous NLRC5 expression markedly enhances the expression of the type I IFN-dependent genes ISG54 and ISG56, thus increasing both innate and antiviral immune responses.

### Example 8

### Significance of Certain Embodiments of the Invention

Innate immunity is the first line of defense against invading microorganisms (Medzhitov and Janeway, 1997). Activation of innate immune receptors TLRs, NLRs and RLRs by their corresponding ligands initiates several key signaling pathways and produces proinflammatory cytokines, such as IL-6 and TNF-α, which in turn induce profound positive feedback on adaptive immune responses (Akira et al., 2006; Honda and Taniguchi, 2006). If left unchecked, innate immune responses can be highly detrimental, even fatal, to the host (Liew et al., 2005). Increasing evidence indicates that many inflammation-associated diseases, including Crohn's and inflammatory bowel diseases, may result from dysregulated innate immunity (Kanneganti et al., 2007; Kobayashi et al., 2002; Ting et al., 2006). More recently, IL-1, IL-6 and TNF-α produced by innate immune cells in chronic inflammation conditions have been shown to promote cancer development and progression (Karin et al., 2006; Kim et al., 2009). Thus, an understanding of the molecular mechanisms by which innate immunity is held in check through negative regulators appears critical for developing novel and more effective treatments for inflammation-induced autoimmune diseases and cancer (Wang et al., 2008).

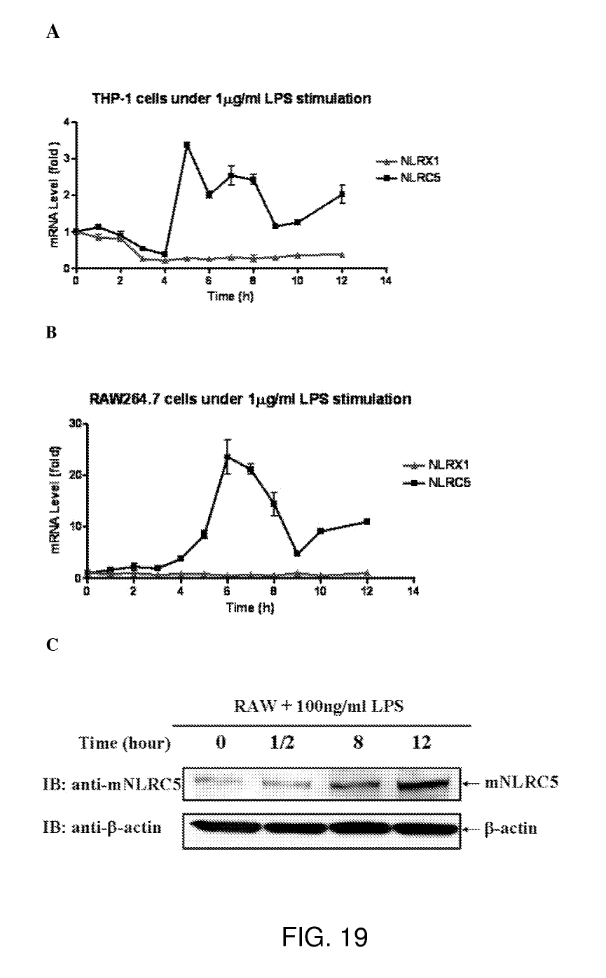

Since the NF-κB and type I interferon signaling pathways control genes involved in apoptosis, inflammation and immunity, their tight regulation is crucial (Liew et al., 2005). Indeed, both NF-κB and type I interferon signaling are controlled at multiple levels by distinct mechanisms, and many such regulatory proteins are themselves direct transcriptional targets of NF-κB and type I interferon signaling, contributing to negative feedback regulation (Komuro et al., 2008). For example, expression of the A20, CYLD and DUBA negative regulators is controlled by NF-κB activity, while the RIG-I and MDA5 genes are transcriptionally regulated by type I interferon signaling. The upregulation of the mRNA level of NLRC5 or mNLRC5 was observed in both human and mouse cells after 6 h of treatment with LPS (FIG. 19).

It has been demonstrated that the deubiquitinating enzyme A20 inhibits NF-κB signaling by targeting TRAF6 upstream of IKK (Kovalenko et al., 2003; Liew et al., 2005; Trompouki et al., 2003; Wertz et al., 2004), while the deubiquitinating protein DUBA inhibits type I interferon activity by targeting TRAF3 (Kayagaki et al., 2007), a critical adaptor molecule required for the type I interferon response (Hacker et al., 2005; Oganesyan et al., 2005). The present invention supports a dual regulatory role for NLRC5 that encompasses both NF-κB and type I interferon signaling. By interacting with IKKα and IKKβ but not with the regulatory subunit NEMO, NLRC5 blocks the phosphorylation of these molecules and thus NF-κB activation. It also, and perhaps more importantly, specifically interacts with RIG-I and MDA5, key cytosolic receptors for many RNA viruses such as VSV, but not with MAVS, TBK1, KKI, TRIF or IRF3 molecules, thus blocking the RLR-mediated type I interferon signaling pathway. This dual and specific inhibitory activity of NLRC5 clearly differs from that of NLRX1, which blocks the type I interferon signaling pathway via interaction with MAVS (Moore et al., 2008).

Among the potential regulatory targets of NLRC5, IKK acts as a central transducer of signaling from cytokines, TLRs, and RLRs to stimulate NF-κB activation. A recent study shows that CUEDC2 and PP1c are involved in the deactivation of the IKK complex by dephosphorylating IKKα and IKKIβ (Li et al., 2008), whereas the results herein show that NLRC5 specifically binds to the kinase domain of IKKβ and inhibits its phosphorylation. To understand how NLRC5 exerts its inhibitory effect on the kinase domain of IKKβ, a series of deletion experiments were conducted which indicated that the LRR-R2 domain (NLRC5-D3) not only binds to the kinase domain of IKKβ, but also inhibits the phosphorylation of IKK complexes. By contrast, deletion of several other domains resulted in little or no inhibitory activity against IKK phosphorylation, even though each of the deletion mutants was able to bind to the IKKIβ kinase domain. Because RIG-I and MDA-5 are key receptors for triggering type I interferon signaling pathways, and are controlled by positive and negative regulators, such as TRIM25 and RNF125, through ubiquitination (Arimoto et al., 2007; Gack et al., 2007), in certain aspects other regulatory proteins are likely involved in the regulation of RLRs. Results presented here show that NLRC5 is such a protein, capable of binding to both RIG-I and MDA5 but not to MAVS.

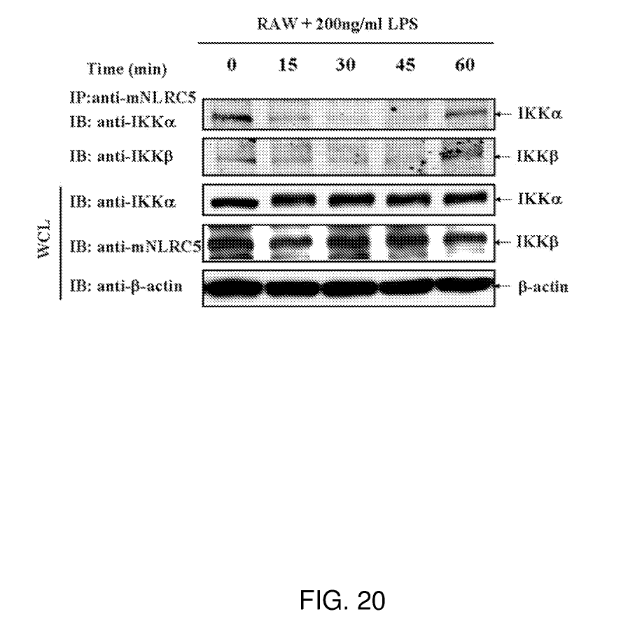

To further understand how NLRC5 controls the activation of NF-κB and type I interferon signaling under physiological conditions, studies were performed before and after stimulation with LPS or viral infection. The results indicate that NLRC5 is associated with IKKα and IKKIβ before stimulation, but disassociates from these kinases immediately after stimulation, reassociating with both molecules within 60 min (FIG. 20). By contrast, NLRC5 does not bind to RIG-1 until 6 h after VSV-eGFP infection, indicating that it relies on an entirely different mechanism to negatively regulate these critical signaling pathways.

Based on the findings, in specific embodiments of the invention there is a model by which NLRC5 negatively controls both NF-κB and type I interferon signaling, as shown in FIG. 16E. NLRC5 constitutively inactivates IKKα and IKKβ by blocking their phosphorylation, but upon immune stimulation, the kinases are released from the influence of NLRC5, permitting their phosphorylation by upstream signaling molecules. Importantly, NLRC5 reassociates with IKKα and IKKβ in 1 h (after they phosphorylate IκB), restoring their inactive state. Because the NLRC5 gene itself is a direct target of NF-κB signaling, its expression increases as immune stimulation progresses (FIG. 19) allowing the protein to effectively compete with NEMO to inhibit the phosphorylation of IKKα and IKKβ. Similarly, viral activation of RLRs triggers both type I interferon and NF-κB signaling, which upregulates NLRC5 expression, resulting in turn in negative feedback inhibition of both pathways.

Consistent with this model, NLRC5 knockdown remarkably amplified NF-κB and type I interferon responses, and enhanced the secretion of proinflammatory cytokines, such as IL-6 and TNF-α, by numerous human and mouse cell types. More importantly, specific knockdown of NLRC5 led to enhanced antiviral immunity in multiple cell lines, similar to NLRX1 knockdown (Moore et al., 2008). Taken together, these results indicate that the biological function of NLRC5 is conserved in humans and mice, as well as in specific cell types, indicating a physiologically important role of this molecule in maintenance of the innate immune system. Precise elucidation of the in vivo function of NLRC5 in inflammation and antiviral immunity requires further study in NLRC5 knockout mice.

In summary, NLRC5 is a negative regulator of both the NF-κB and type I interferon signaling pathways, acting through inhibition of IKKα/β phosphorylation as well as interaction with RIG-I and MDA5. Knockdown of NLRC5 enhances NF-κB and type I interferon activity and produces large amounts of inflammatory cytokines, such as IL-6, TNF-α and IFN-β, leading to enhanced antiviral immunity. NLRC5 therefore provides a useful therapeutic target for enhancing immunity against microbial infections and inflammation-associated diseases.

### Example 9

### Exemplary NLRC5 Studies

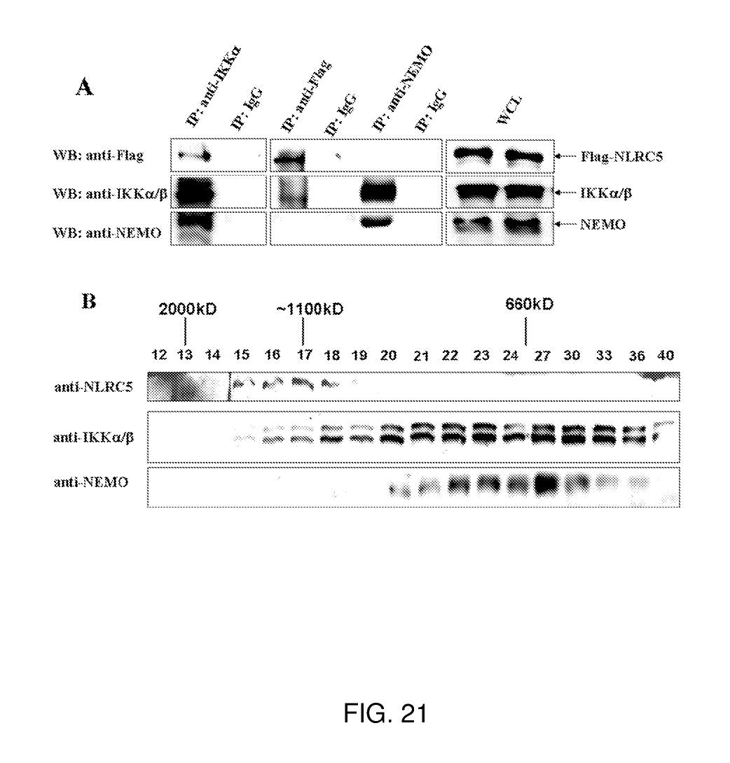

FIG. 21 demonstrates that NLRC5/IKKα/β complex and IKKα/β/NEMO complex co-exist in resting cells. (A) Cell extracts of HEK293T cells expressing Flag-NLRC5 were immunoprecipitated with anti-IKKα, anti-Flag, anti-NEMO or control IgG antibody, and then immunoblotted with the indicated antibodies. (B). Cell extracts of RAW264.7 cells were fractionated on a size-exclusion column (HiPrep 16/60 Sephacryl S-300 HR). Factions collected were resolved on SDS-polyacrylamide gel electrophoresis and immunoblotted with anti-mNLRC5, anti-IKKα/β or anti-NEMO antibody.

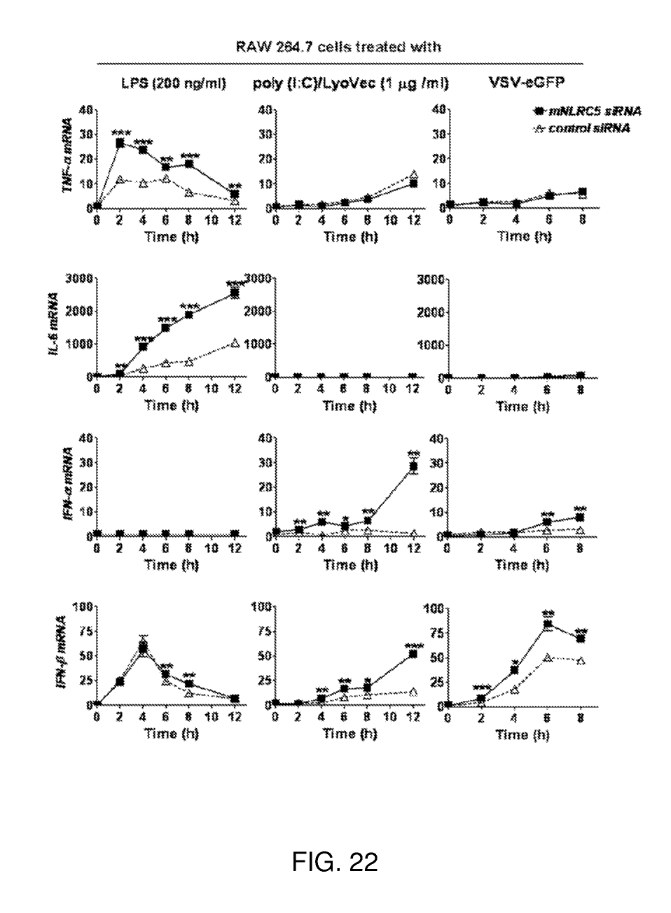

FIG. 22 shows that RAW264.7 cells were transfected with mNLRC5 siRNA or scrambled siRNA for 36 h and then treated with LPS (200 ng/ml), poly (I:C)/LyoVec (1 μg/ml) or VSV-eGFP infection. Total RNA from the treated cells was harvested at different time points and used for realtime PCR analysis for detecting the gene expression of TNF-α, IL-6, IFN-α and IFN-β.

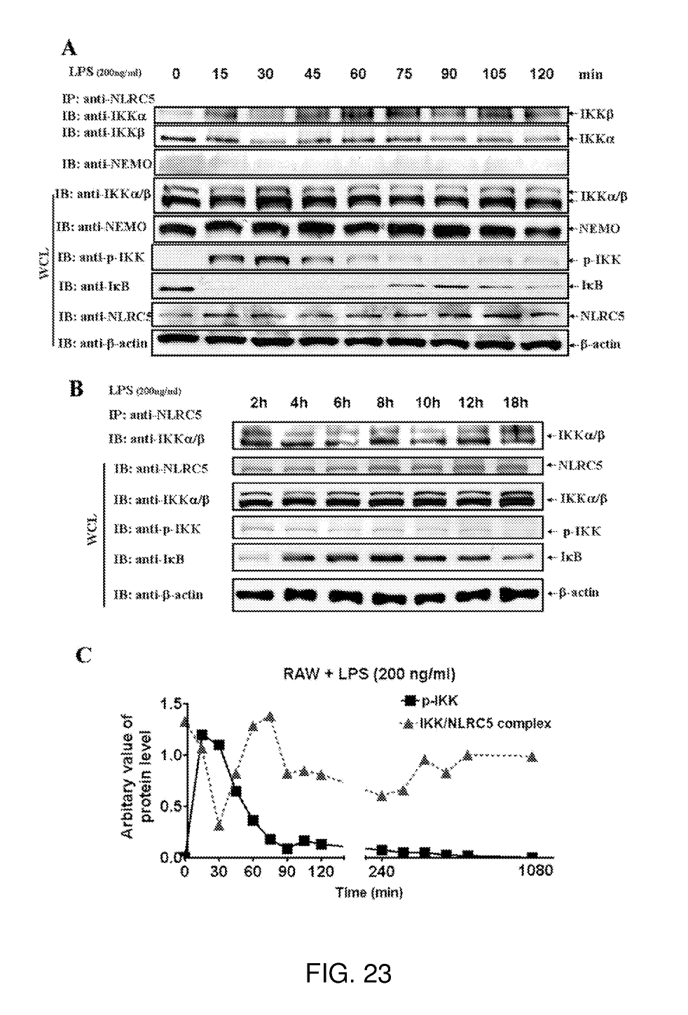

FIG. 23 demonstrates dynamic interaction between NLRC5 and IKKα/IKKβ over time after LPS stimulation. (A) RAW264.7 cells were treated with 200 ng/ml LPS and total cell lysates were collected at the indicated time for immunoprecipitation with anti-mNLRC5, followed by immunoblotting with anti-IKKα, anti-IKKβ and anti-NEMO antibody. Whole cell lysates were immunoblotted with the indicated antibodies. (B) The interaction between NLRC5 and IKKα/IKKβ over period 2-18 h after treatment. (C) Analysis of band density in 23A and 23B for p-IKK and NLRC/IKK pulled down by anti-NLRC5 (i.e. NLRC5/IKK complex).

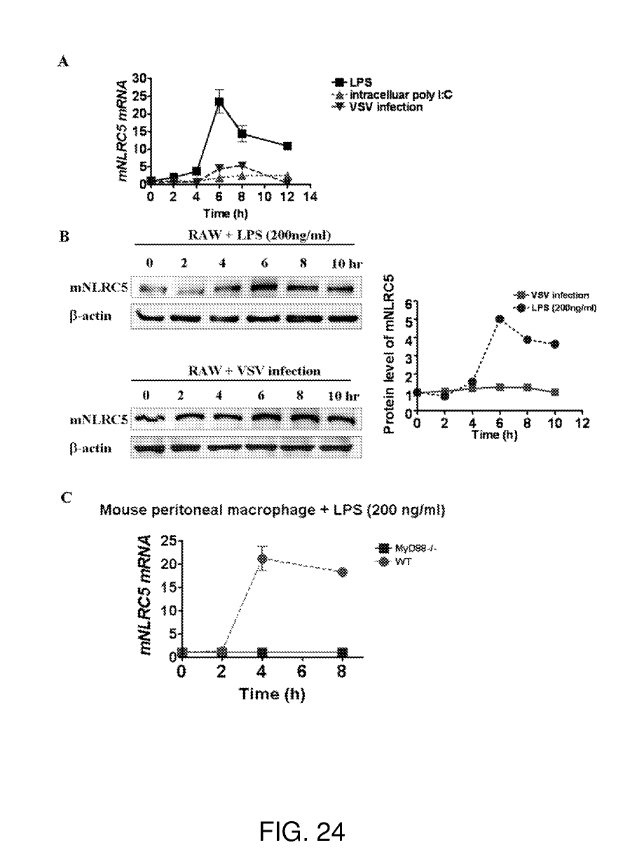

FIG. 24 shows that RAW264.7 cells were treated with LPS (200 ng/ml), poly (I:C)/LyoVec and VSV-eGFP. Cell extracts and RNA were collected for further analysis. (A) Realtime PCR was performed to determine the mRNA expression level of mNLRC5 at indicated time points. (B) mNLRC5 and beta-actin were immunoblotted by anti-mNLRC5 and anti-β-actin after treatment. The density of bands was scanned and analyzed by B and Scan software. The band density of β-actin was used to normalize the data. (C). MyD88−/− or wild type mouse peritoneal macrophages were treated with LPS (200 ng/ml) and RNA were collected for real-time PCR analysis.

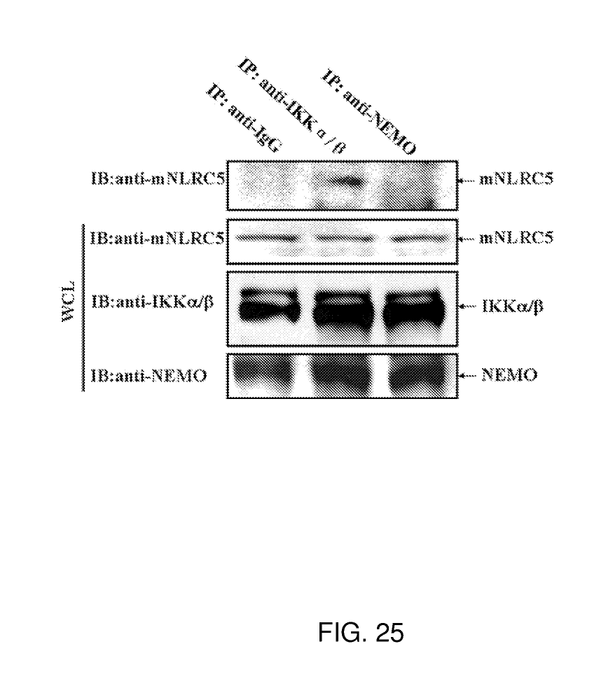

FIG. 25 shows endogenous mNLRC5 interacts with IKKα/β. RAW264.7 cell extracts were immunoprecipitated with anti-IKKα/β, anti-NEMO or control IgG, respectively, and then analyzed by Western blot with an anti-NLRC5 or mNLRC5 antibody.

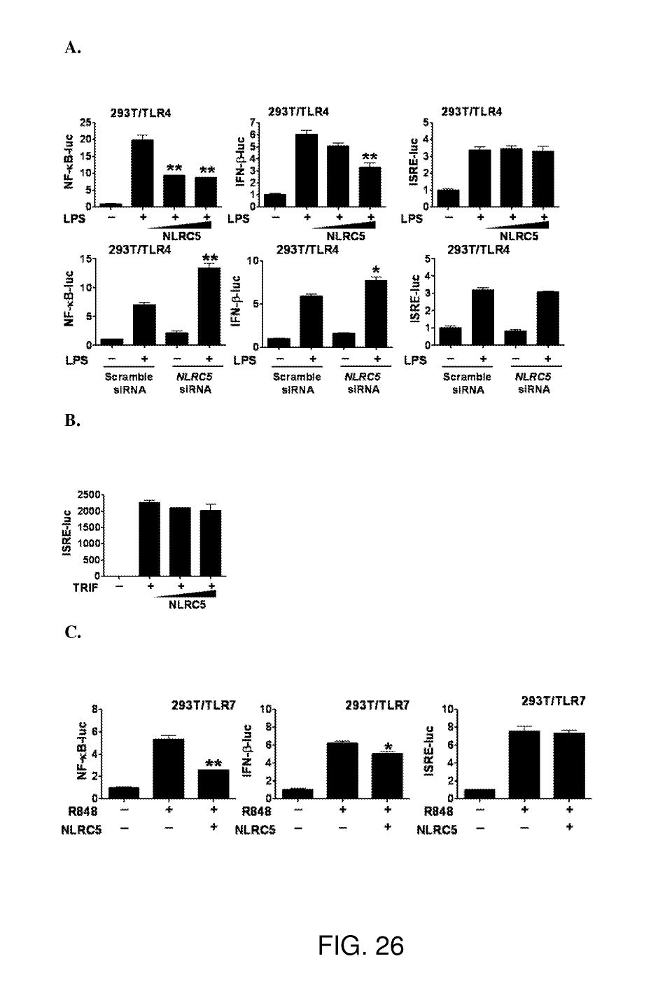

FIG. 26. (A). Inhibition of NF-κB-luc, IFN-β^{˜}-luc and ISRE-luc activity by LPS treatment in 293T/TLR4 in the presence of increasing doses (0, 100 and 200 ng) of NLRC5 or NLRC5 siRNAs. (B) NLRC5 can not inhibit ISRE-luc activity induced by overexpression of TRIF in 293T cells. (C). Inhibition of NF-κB-luc, IFN-β^{˜}-luc and ISRE-luc activity by R848 treatment in 293T/TLR7 of NLRC5 (0 or 200 ng).

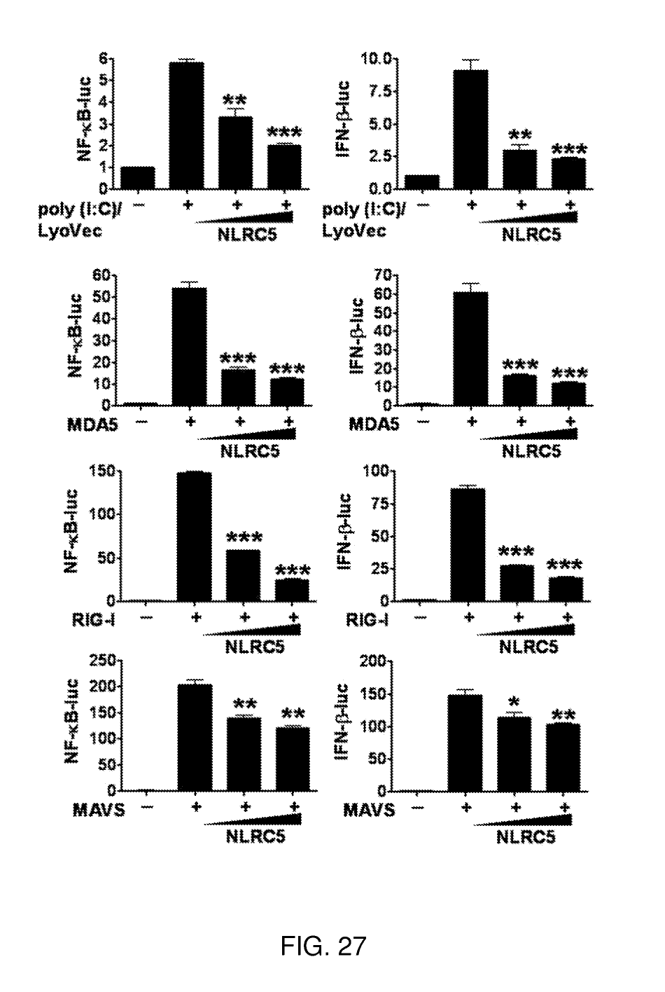

FIG. 27 shows that NLRC5 inhibits NF-κB-luc and IFN-β-luc activity induced by poly (I:C)/LyoVec treatment or overexpression of MDA5, RIG-I or MAVS in 293T cells.

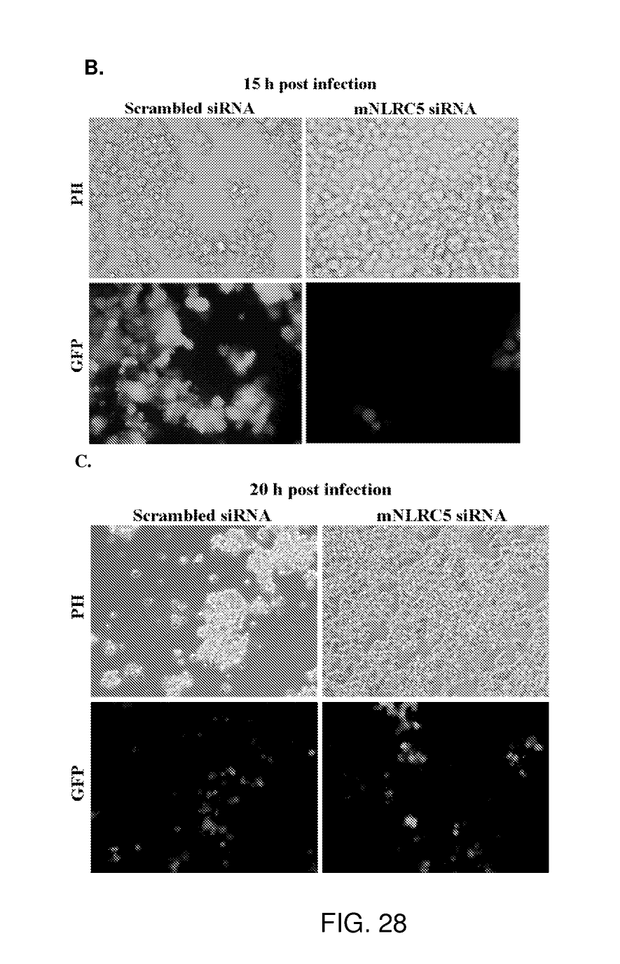

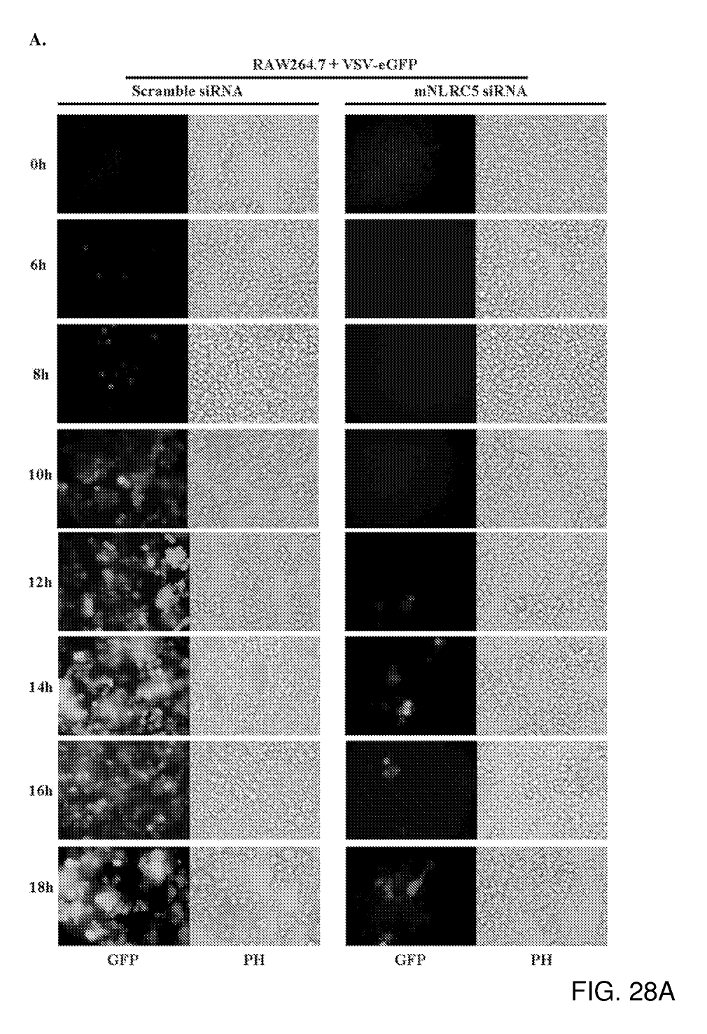

FIG. 28 shows that RAW264.7 cells were transfected with scrambled siRNA or mNLRC5 siRNA for 36 h and then infected with VSV-eGFP. The images of fluorescence and phase-contrast were taken at 0, 6, 8, 10, 12, 14, 16, 18 h (A), 15 h (B), 20 h (C) to monitor the propagations of VSV-eGFP.

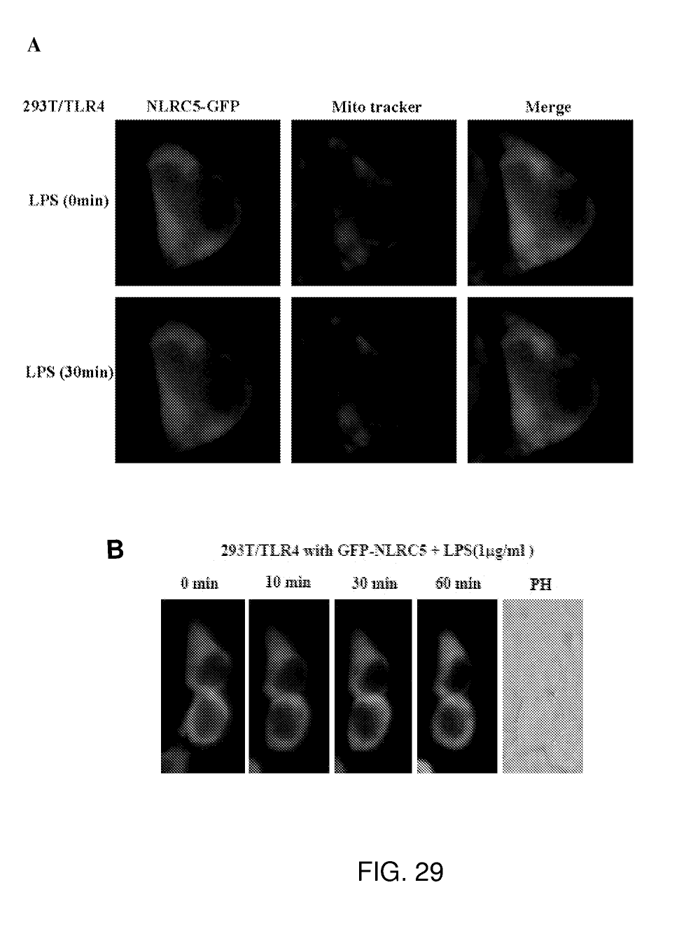

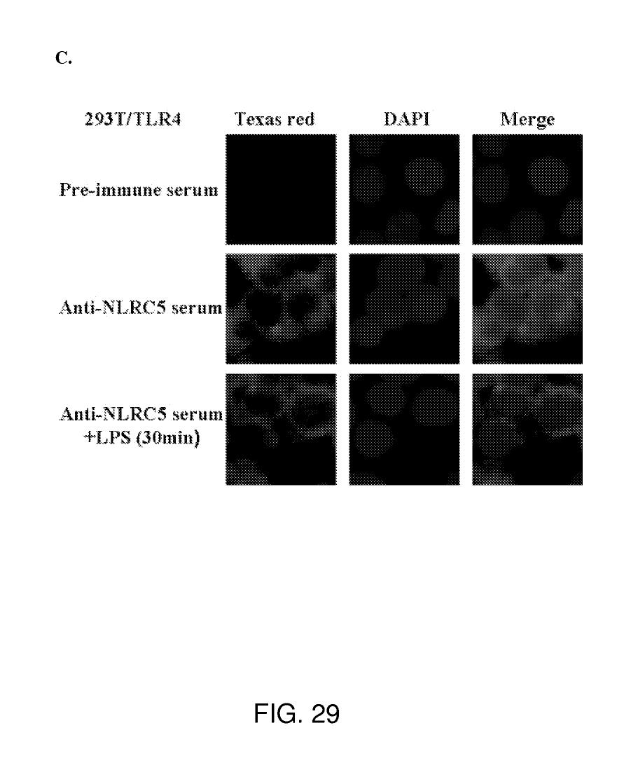

FIG. 29 demonstrates that 293T/TLR4 cells were transfected with NLRC5-GFP for 24 h and treated with 1 μg/ml LPS. Mito-tracker was used to stain mitochondria (A) and GFP-NLRC5 distribution was monitored within 1 h (B). (C) 293T/TLR4 cells were treated with 1 μg/ml LPS. NLRC5 polyclone antibody was used to detect endogenous NLRC5.

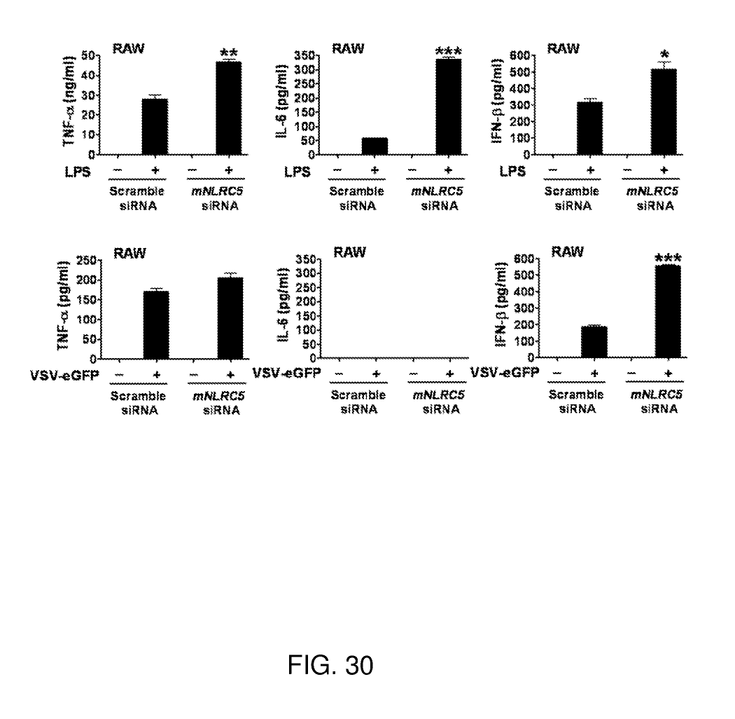

FIG. 30 shows that RAW264.7 cells were transfected with scrambled siRNA or mNLRC5 siRNA for 36 h and treated with LPS (200 ng/ml) or VSV-eGFP. Cell supernatants were collected at 10 h to measure cytokine production with ELISA kits.

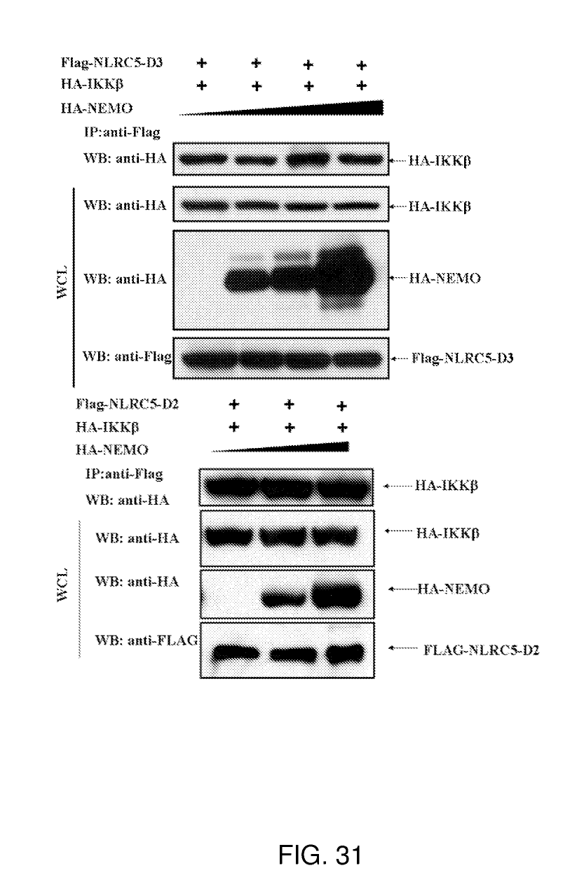

FIG. 31 demonstrates competitive binding between NLRC5-D3 or NLRC5-D2 and NEMO to IKKβ. 293T cells were transfected with indicated doses of Flag-NLRC5-D3 or Flag-NLRC5-D2, HA-IKKβ and HA-NEMO.

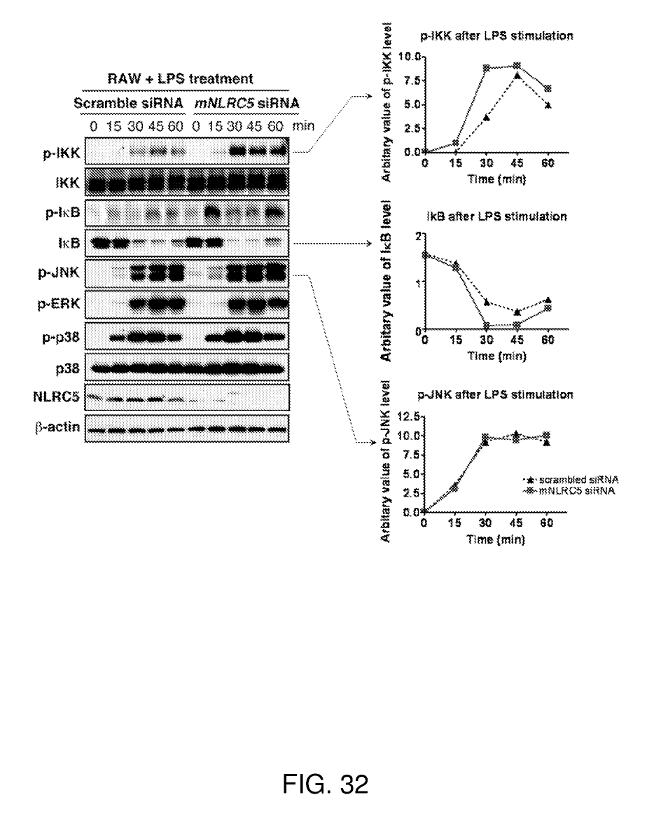

FIG. 32 shows analysis of band density in FIG. 10B. (A) p-IKK, (B) IκB, (C) p-JNK. All the data were normalized by β-actin and p38.

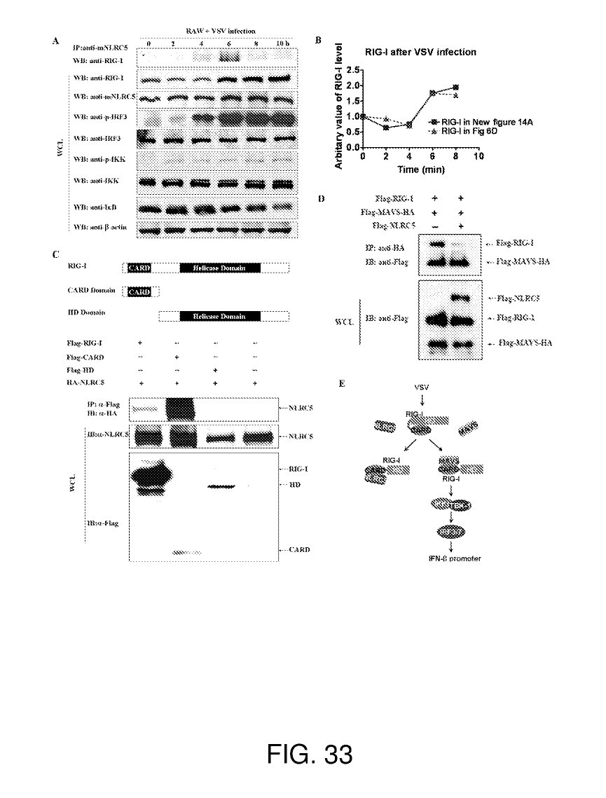

FIG. 33 (A) RAW264.7 were infected with VSV-eGFP and cell extracts were collected at different time points, immunoprecipitated with anti-mNLRC5 antibody and analyzed by Western blot with the indicated antibodies. (B) Analysis of band density of RIG-I in FIG. 13D and FIG. 33A. All the data were normalized by β-actin. (C) 293T cells were transfected with HA-NLRC5 plus Flag-RIG-I, Flag-RIG-I CARD domain and Helicase domain (HD). After immunoprecipitation with anti-Flag beads, specific proteins were analyzed by Western blot with anti-HA. (D) HEK293T cells were transfected with Flag-MAVS-HA plus Flag-RIG-1, or Flag-NLRC5. After immunoprecipitation with anti-HA beads, specific proteins were analyzed by western blot with anti-Flag. (E) Proposed model of the negative regulation of RIG-I activation by NLRC5.

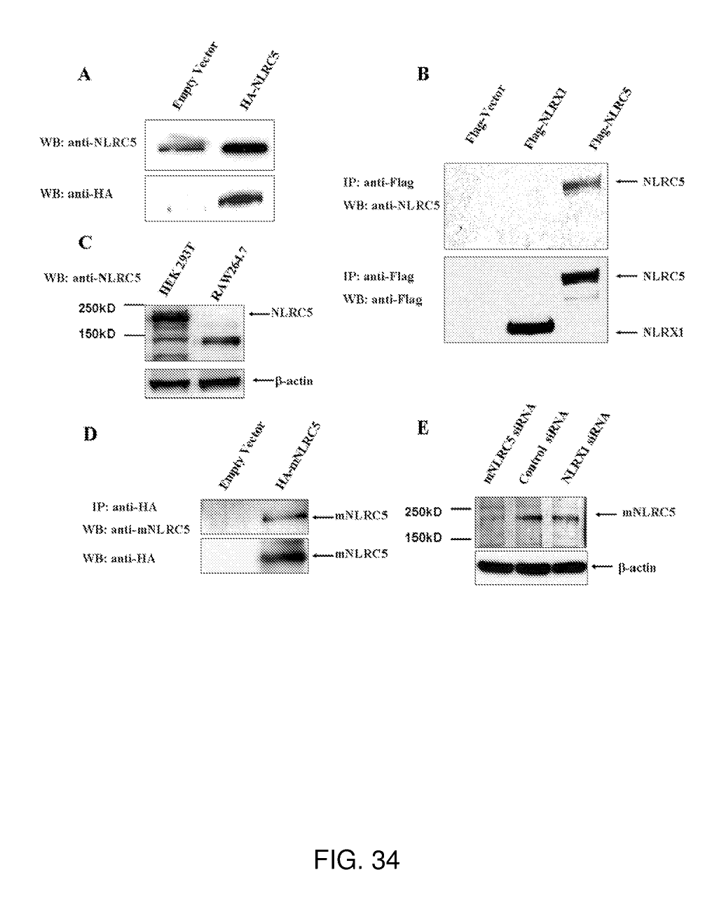

FIG. 34 (A). Anti-NLRC5 can recognize endogenous NLRC5 and HA-NLRC5. (B). Anti-NLRC5 can specifically recognize Flag-NLRC5 but not Flag-NLRX1. (C). Anti-NLRC5 can not recognize mNLRC5 in RAW264.7 cells. (D). anti-mNLRC5 can recognize HA-mNLRC5. (E). Anti-mNLRC5 can specifically recognize endogenous mNLRC5 in RAW264.7 cells. Specific knockdown of endogenous mNLRC5 expression by mNLRC5 siRNA but not by NLRX1 siRNAs in RAW264.7 cells clearly reduced the band detected by anti-NLRC5.

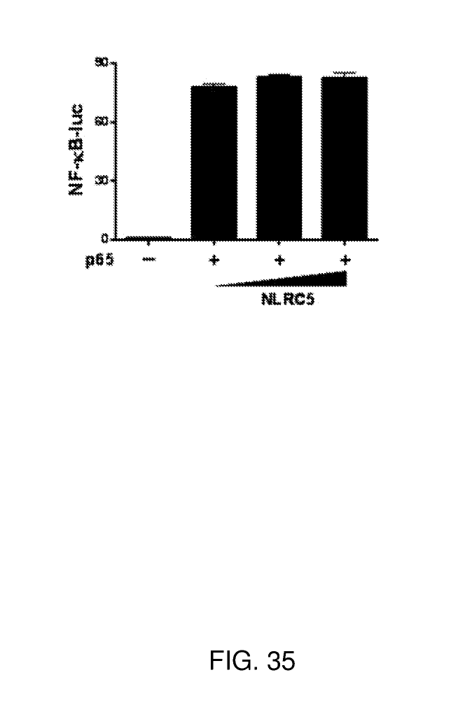

FIG. 35 shows that 293T cells were transfected with 1 ng p65 and increasing doses of NLRC5 (0, 50, 100, 200 ng), NF-κB-luc and TK-Renilla-luc for 24 h, and then analyzed by luciferase assay.

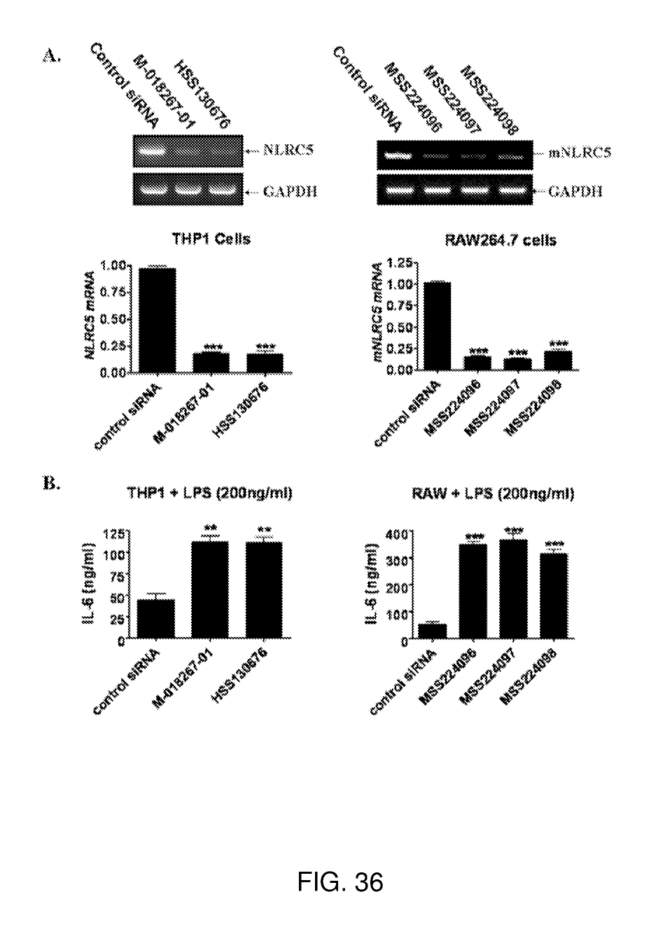

FIG. 36 shows that RAW264.7 or THP-1 cells were transfected with the indicated siRNA for 36 h. Total RNA were collected for RT-PCR and real-time-PCR analysis (A). 36 h post transfection of siRNAs, RAW264.7 and THP-1 cells were treated with 200 ng/ml LPS for 6 h and medium was collected for ELISA analysis (B).

### Example 10

### Exemplary Experimental Procedures

### Molecular Cloning of Full-Length Human and Mouse NLRC5

A complete open reading frame of human NLRC5 was obtained from human PBMC cDNA by two-step PCR. The 3.5 kb N-terminal and 2.1 kb C-terminal PCR fragments were cloned into pcDNA3.1Z with a HA tag sequence. The resultant plasmid DNA was sequenced to verify the correct DNA sequence and open reading frame. A similar strategy was used to clone N-terminal and C-terminal fragments of mouse NLRC5. Both of the fragments were subsequently subcloned into pcDNA3.1HA using EcoRV and XbaI. The final product, pcDNA-HA-mNLRC5, was sequenced to verify the correct DNA sequence and open reading frame.

### Expression Profile and Antibody Production

The expression profile of NLRC5 and mNLRC5 in different tissues was evaluated by reverse transcriptase (RT)-PCR analysis. NLRC5 and mNLRC5 peptides were used to generate polyclonal antibodies by standard methods.

### Luciferase Assays, Immunoprecipitation and Western Blot Analysis

HEK293 cells were transfected with IFN-β, NF-κB luciferase plasmids and HA-NLRC5. TNF-α, IL-1β, LPS as well as exogenous MyD88, TRAF6, IKKα, IKKβ, NEMO, p65 (NF-κB), RIG-I, MDA5, MAVS and IKKi plasmids were used as stimulators. All co-immunoprecipitation and Western blots were performed by standard methods. 293T cells transfected with or without specific plasmids were used as starting materials for immunoprecipitation. Specific protein expression was detected by anti-HA, anti-Flag or specific antibodies.

### Electrophoretic Mobility Shift Assay (EMSA)

Electrophoretic mobility shift assays were performed by using the LightShift Chemiluminescent EMSA kit from Pierce Biotechnology according to the manufacturer's standard protocol.

### Real-Time PCR Analysis

First-strand cDNA was generated from total RNA using oligo-dT primers and reverse transcriptase (Invitrogen). Real-time PCR was conducted with the QuantiTect SYBR Green PCR Master Mix (Qiagen) and specific primers on an ABI Prism 7000 analyzer (Applied Biosystems).

Knockdown of NLRC5 and mNLRC5 by RNA Interference

NLRC5-specific, mNLRC5-specific and control (2-scramble mix) siRNA oligonucleotides were obtained from Invitrogen and Integrated DNA Technologies, and transfected into 293T, THP-1, RAW264.7 and primary cells with use of Lipofactamine 2000 (Invitrogen) and various Nucleofector kits (one for each cell type) according to the manufacturer's instruction.

### Cytokine Production and ELISA

DuoSet ELISA Kits (R&D Systems) were employed to detect both human and mouse TNF-α and IL-6 secretion, and IFN-β was measured by ELISA kits from PBL Biomedical Laboratories, following the manufacturer's standard protocols.

Virus infection. VSV-eGFP virus was provided by S. Balachandran, and infected various types of cells at different multiplicity of infections.

### REFERENCES

All patents and publications mentioned in the specification are indicative of the levels of those skilled in the art to which the invention pertains. All patents and publications are herein incorporated by reference to the same extent as if each individual publication was specifically and individually indicated to be incorporated by reference.
Akira, S., Uematsu, S., and Takeuchi, O. (2006). Pathogen recognition and innate immunity. Cell 124, 783-801.
Arimoto, K., Takahashi, H., Hishiki, T., Konishi, H., Fujita, T., and Shimotohno, K. (2007). Negative regulation of the RIG-I signaling by the ubiquitin ligase RNF125. Proc Natl Acad Sci USA 104, 7500-7505.
Chen, Z. J. (2005). Ubiquitin signalling in the NF-kappaB pathway. Nat Cell Biol 7, 758-765.
Dostert, C., Petrilli, V., Van Bruggen, R., Steele, C., Mossman, B. T., and Tschopp, J. (2008). Innate immune activation through Nalp3 inflammasome sensing of asbestos and silica. Science 320, 674-677.
Dowds, T. A., Masumoto, J., Chen, F. F., Ogura, Y., Inohara, N., and Nunez, G. (2003). Regulation of cryopyrin/Pypaf1 signaling by pyrin, the familial Mediterranean fever gene product. Biochem Biophys Res Commun 302, 575-580.
Eisenbarth, S. C., Colegio, O. R., O'Connor, W., Sutterwala, F. S., and Flavell, R. A. (2008). Crucial role for the Nalp3 inflammasome in the immunostimulatory properties of aluminium adjuvants. Nature 453, 1122-1126.
Gack, M. U., Shin, Y. C., Joo, C. H., Urano, T., Liang, C., Sun, L., Takeuchi, O., Akira, S., Chen, Z., Inoue, S., et al. (2007). TRIM25 RING-finger E3 ubiquitin ligase is essential for RIG-1-mediated antiviral activity. Nature 446, 916-920.
Hacker, H., and Karin, M. (2006). Regulation and function of IKK and IKK-related kinases. Sci STKE 2006, re13.
Hacker, H., Redecke, V., Blagoev, B., Kratchmarova, I., Hsu, L. C., Wang, G. G., Kamps, M. P., Raz, E., Wagner, H., Hacker, G., et al. (2005). Specificity in Toll-like receptor signalling through distinct effector functions of TRAF3 and TRAF6. Nature.
Hayden, M. S., and Ghosh, S. (2008). Shared principles in NF-kappaB signaling. Cell 132, 344-362.
Honda, K., and Taniguchi, T. (2006). IRFs: master regulators of signalling by Toll-like receptors and cytosolic pattern-recognition receptors. Nat Rev Immunol 6, 644-658.
Hornung, V., Ellegast, J., Kim, S., Brzozka, K., Jung, A., Kato, H., Poeck, H., Akira, S., Conzelmann, K. K., Schlee, M., et al. (2006). 5′-Triphosphate RNA is the ligand for RIG-I. Science 314, 994-997.
Inohara, Chamaillard, McDonald, C., and Nunez, G. (2005). NOD-LRR proteins: role in host-microbial interactions and inflammatory disease. Annu Rev Biochem 74, 355-383.
Kanneganti, T. D., Lamkanfi, M., and Nunez, G. (2007). Intracellular NOD-like receptors in host defense and disease. Immunity 27, 549-559.
Karin, M., Lawrence, T., and Nizet, V. (2006). Innate immunity gone awry: linking microbial infections to chronic inflammation and cancer. Cell 124, 823-835.
Kato, H., Takeuchi, O., Sato, S., Yoneyama, M., Yamamoto, M., Matsui, K., Uematsu, S., Jung, A., Kawai, T., Ishii, K. J., et al. (2006). Differential roles of MDA5 and RIG-I helicases in the recognition of RNA viruses. Nature 441, 101-105.
Kawai, T., Takahashi, K., Sato, S., Coban, C., Kumar, H., Kato, H., Ishii, K. J., Takeuchi, O., and Akira, S. (2005). IPS-1, an adaptor triggering RIG-1- and MdaS-mediated type I interferon induction. Nat Immunol 6, 981-988.
Kayagaki, N., Phung, Q., Chan, S., Chaudhari, R., Quan, C., O'Rourke, K. M., Eby, M., Pietras, E., Cheng, G., Bazan, J. F., et al. (2007). DUBA: a deubiquitinase that regulates type I interferon production. Science 318, 1628-1632.
Kim, S., Takahashi, H., Lin, W. W., Descargues, P., Grivennikov, S., Kim, Y., Luo, J. L., and Karin, M. (2009). Carcinoma-produced factors activate myeloid cells through TLR2 to stimulate metastasis. Nature 457, 102-106.
Kobayashi, K., Hernandez, L. D., Galan, J. E., Janeway, C. A., Jr., Medzhitov, R., and Flavell, R. A. (2002). IRAK-M is a negative regulator of Toll-like receptor signaling. Cell 110, 191-202.
Kobayashi, K. S., Chamaillard, M., Ogura, Y., Henegariu, O., Inohara, N., Nunez, G., and Flavell, R. A. (2005). Nod2-dependent regulation of innate and adaptive immunity in the intestinal tract. Science 307, 731-734.
Komuro, A., Bamming, D., and Horvath, C. M. (2008). Negative regulation of cytoplasmic RNA-mediated antiviral signaling. Cytokine 43, 350-358.
Kovalenko, A., Chable-Bessia, C., Cantarella, G., Israel, A., Wallach, D., and Courtois, G. (2003). The tumour suppressor CYLD negatively regulates NF-kappaB signalling by deubiquitination. Nature 424, 801-805.
Li, H. Y., Liu, H., Wang, C. H., Zhang, J. Y., Man, J. H., Gao, Y. F., Zhang, P. J., Li, W. H., Zhao, J., Pan, X., et al. (2008). Deactivation of the kinase IKK by CUEDC2 through recruitment of the phosphatase PP1. Nat Immunol 9, 533-541.
Liew, F. Y., Xu, D., Brint, E. K., and O'Neill, L. A. (2005). Negative regulation of toll-like receptor-mediated immune responses. Nat Rev Immunol 5, 446-458.
Liu, H., Komai-Koma, M., Xu, D., and Liew, F. Y. (2006). Toll-like receptor 2 signaling modulates the functions of CD4+CD25+ regulatory T cells. Proc Natl Acad Sci USA 103, 7048-7053.
Medzhitov, R., and Janeway, C. A., Jr. (1997). Innate immunity: impact on the adaptive immune response. Curr Opin Immunol 9, 4-9.
Meylan, E., Curran, J., Hofmann, K., Moradpour, D., Binder, M., Bartenschlager, R., and Tschopp, J. (2005). Cardif is an adaptor protein in the RIG-I antiviral pathway and is targeted by hepatitis C virus. Nature 437, 1167-1172.
Meylan, E., Tschopp, J., and Karin, M. (2006). Intracellular pattern recognition receptors in the host response. Nature 442, 39-44.
Moore, C. B., Bergstralh, D. T., Duncan, J. A., Lei, Y., Morrison, T. E., Zimmermann, A. G., Accavitti-Loper, M. A., Madden, V. J., Sun, L., Ye, Z., et al. (2008). NLRX1 is a regulator of mitochondrial antiviral immunity. Nature 451, 573-577.
Oganesyan, G., Saha, S. K., Guo, B., He, J. Q., Shahangian, A., Zarnegar, B., Perry, A., and Cheng, G. (2005). Critical role of TRAF3 in the Toll-like receptor-dependent and -independent antiviral response. Nature.
Opitz, B., Vinzing, M., van Laak, V., Schmeck, B., Heine, G., Gunther, S., Preissner, R., Slevogt, H., N′Guessan, P. D., Eitel, J., et al. (2006). Legionella pneumophila induces IFNbeta in lung epithelial cells via IPS-1 and IRF3, which also control bacterial replication. J Biol Chem 281, 36173-36179.
Pasare, C., and Medzhitov, R. (2003). Toll Pathway-Dependent Blockade of CD4+CD25+T Cell-Mediated Suppression by Dendritic Cells. Science 299, 1033-1036.
Peng, G., Guo, Z., Kiniwa, Y., Voo, K. S., Peng, W., Fu, T., Wang, D. Y., Li, Y., Wang, H. Y., and Wang, R.-F. (2005). Toll-like receptor 8 mediated-reversal of CD4+ regulatory T cell function. Science 309, 1380-1384.
Peng, G., Wang, H. Y., Peng, W., Kiniwa, Y., Seo, K., and Wang, R.-F. (2007). Tumor-infiltrating gamma-delta T cells suppress T and dendritic cell function via mechanisms controlled by a unique Toll-like receptor signaling pathway. Immunity 27, 334-348.
Pichlmair, A., Schulz, O., Tan, C. P., Naslund, T. I., Liljestrom, P., Weber, F., and Reis e Sousa, C. (2006). RIG-1-mediated antiviral responses to single-stranded RNA bearing 5′-phosphates. Science 314, 997-1001.
Seth, R. B., Sun, L., Ea, C. K., and Chen, Z. J. (2005). Identification and characterization of MAVS, a mitochondrial antiviral signaling protein that activates NF-kappaB and IRF 3. Cell 122, 669-682.
Shaw, M. H., Reimer, T., Kim, Y. G., and Nunez, G. (2008). NOD-like receptors (NLRs): bona fide intracellular microbial sensors. Curr Opin Immunol 20, 377-382.
Sutmuller, R. P., den Brok, M. H., Kramer, M., Bennink, E. J., Toonen, L. W., Kullberg, B. J., Joosten, L. A., Akira, S., Netea, M. G., and Adema, G. J. (2006). Toll-like receptor 2 controls expansion and function of regulatory T cells. J Clin Invest 116, 485-494.
Tattoli, I., Carneiro, L. A., Jehanno, M., Magalhaes, J. G., Shu, Y., Philpott, D. J., Arnoult, D., and Girardin, S. E. (2008). NLRX1 is a mitochondrial NOD-like receptor that amplifies NF-kappaB and JNK pathways by inducing reactive oxygen species production. EMBO Rep 9, 293-300.
Ting, J. P., and Davis, B. K. (2005). CATERPILLER: a novel gene family important in immunity, cell death, and diseases. Annu Rev Immunol 23, 387-414.
Ting, J. P., Kastner, D. L., and Hoffman, H. M. (2006). CATERPILLERs, pyrin and hereditary immunological disorders. Nat Rev Immunol 6, 183-195.

Trompouki, E., Hatzivassiliou, E., Tsichritzis, T., Farmer, H., Ashworth, A., and Mosialos, G. (2003). CYLD is a deubiquitinating enzyme that negatively regulates NF-kappaB activation by TNFR family members. Nature 424, 793-796.
Wang, R. F., Miyahara, Y., and Wang, H. Y. (2008). Toll-like receptors and immune regulation: implications for cancer therapy. Oncogene 27, 181-189.
Wertz, I.E., O'Rourke, K. M., Zhou, H., Eby, M., Aravind, L., Seshagiri, S., Wu, P., Wiesmann, C., Baker, R., Boone, D. L., et al. (2004). De-ubiquitination and ubiquitin ligase domains of A20 downregulate NF-kappaB signalling. Nature 430, 694-699.
Xu, L. G., Wang, Y. Y., Han, K. J., Li, L. Y., Zhai, Z., and Shu, H. B. (2005). VISA is an adapter protein required for virus-triggered IFN-beta signaling. Mol Cell 19, 727-740.
Yoneyama, M., Kikuchi, M., Natsukawa, T., Shinobu, N., Imaizumi, T., Miyagishi, M., Taira, K., Akira, S., and Fujita, T. (2004). The RNA helicase RIG-I has an essential function in double-stranded RNA-induced innate antiviral responses. Nat Immunol 5, 730-737.
Zhong, B., Yang, Y., Li, S., Wang, Y. Y., Li, Y., Diao, F., Lei, C., He, X., Zhang, L., Tien, P., et al. (2008). The adaptor protein MITA links virus-sensing receptors to IRF3 transcription factor activation. Immunity 29, 538-550.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of enhancing an immune response against microbial infection or inflammation-associated disease in an individual, comprising the step of providing to the individual an agent that inhibits NLRC5 in cells of the individual.

2. The method of claim 1, wherein the agent inhibits NLRC5 mRNA or protein.

3. The method of claim 1, wherein the agent is nucleic acid, polypeptide, or a small molecule.

4. The method of claim 1, wherein the agent is NLRC5 siRNA.

5. The method of claim 4, wherein the NLRC5 siRNA targets at least part of the region of the NLRC5 mRNA that encodes the CARD-like domain, the central NOD domain, the LRR region, the region that interacts with NFκB, the region that interacts with IKKα, or the region that interacts with IKKβ.

6. The method of claim 4, wherein the NLRC5 siRNA targets at least part of the region of amino acids 900-1329 of NLRC5 polypeptide.

7. The method of claim 1, wherein the microbial infection is a bacterial infection.

8. The method of claim 7, wherein the bacterial infection is tuberculosis, pneumonia, foodborne illnesses, tetanus, typhoid fever, diphtheria, syphilis or leprosy.

9. The method of claim 1, wherein the microbial infection is a viral infection.

10. The method of claim 9, wherein the viral infection is HIV, smallpox, influenza, mumps, measles, chickenpox, ebola, meningitis, or rubella.

11. The method of claim 1, wherein the microbial infection is a fungal infection.

12. The method of claim 11, wherein the fungal infection is a yeast infection, jock itch, or athlete's foot.

13. The method of claim 1, wherein the agent is provided to the individual by intradermal, transdermal, transmucosal, parenteral, intracranial, intravenous, intramuscular, intranasal, intracerebrospinal, subcutaneous, percutaneous, intratracheal, intraperitoneal, intratumoral, perfusion, lavage, direct injection, and/or oral administration.

14. The method of claim 1, wherein the agent is delivered to the individual in a dendritic cell.

15. The method of claim 1, wherein the agent is delivered to the individual in a liposome.

16. The method of claim 1, further comprising the step of delivering to the individual an agent that blocks Treg cell function.

17. The method of claim 16, wherein the agent that blocks Treg cell function is a non CpG containing oligonucleotide.

18. The method of claim 17, wherein the oligonucleotide has one or more of the following characteristics:
it is between about 4 and about 15 nucleotides
it comprises a guanine and a nuclease-resistant inter-residue backbone linkage;
it comprises a nuclease-sensitive inter-residue backbone linkage; and
it comprises a guanine and a nuclease-resistant inter-residue backbone linkage connecting the guanine and an adjacent nucleobase.

19. A method of treating an individual for cancer or preventing cancer in an individual, comprising the step of delivering to the individual an agent that increases NLRC5 in cancer cells of the individual.

20. The method of claim 19, wherein the agent is NLRC5 polypeptide.

21. The method of claim 19, wherein the agent is an expression construct that is capable of expressing NLRC5.

22. The method of claim 19, wherein the agent that increases NLRC5 is comprised within a liposome.


SEQUENCE LISTING (13 sequences)

SEQ ID NO: 1 - DNA, 23 bp, Artificial Sequence
  Synthetic primer
aannnnnnnnnnnnnnnnnnntt

SEQ ID NO: 2 - protein, 1744 aa, Canis familiaris
MAFTFLKVLNNMEIDTSLFDEVGAKIIKDLMSKAEKNGVKIILPVDFVTAEKFDEHARTGQATVASGIPAGWMGLDCGPESSKKYAEAVAWAKQIMWNGPVGVSEWEALARGTKALMDEVVKATAWDCITIIGGGDIAICCVKWNTEDKVSHGQLLMDHVSFQLGTTNLWSWLVRLLSKDPEWLNAKVKFFLPKMDLSSGDEAQDTTQRVMLQLRRLHTQGRATWQAFIHCVCMELDVPLDLEVPLLSTWGHGDGLKRPHQSCGSSPRLKQCKNHQLELARRYLKLLRTSAQQRYSRIPGPGQPLAFHQAYIPPILQWSRTTAPFSAQDGAVVGDPKAEDDTDVSIRDLFSTRADKGPRVTVLLGKAGMGKTTLAHRLCQRWAAGQLDRFQALFLFEFRQLNLITSSVTLPQLLFDLYLSPEAGPDSVFQYLEEHAGGVLLIFDGLDQALHSHSSTEPEGPEAPSPALALFSGLCRGTILPGCWVLATSRPGKLPDCLPTEAATVHMWGFDRPRVEEYVGRFFSDQEWQEAALAELRENRHLQSMCAVPALCQVTCLCLHHLLPGNSPGQSAALLPTVTQNYVQMVSTLSPRGYLPPESLLGLSEMALRGLETGKVIFSVGDISPPMVAFGAALGLLTSFCICTGPGHQGIGYAFTHLSLQEFFAALYLMASPKVDKNVLARHVTLNSRWVLRTKARLGLLDHLPTFLAGLASCACRPFLSHVAQQDEMWVSDKQAAVMLALRKLAIRRLTGPKVVALCHCVGETQEPELASLMAQSLPSHLSFHNFPLTYTDLSALTNILRHRNASIHLDFEGCPLEPHCPEALAGCKQIENLSFKSRKCGDAFAEALSKSLPTMESLKKLGLAGCKITARGISHLMQALRLCPQLEEISFQDNQLKDRAVLNIVEVLPSLPRLQKLDLSRSNVSVSTLLCLTKVAVTCPTVKMLQVRLLQCQLGIHDVQELIAQLREGPRLEEVNLSNNGLSVDRVHCMLSAVNTCQNLAELHISLLHKTVVFTFAPEQEEQEGIWKRTALRDSLTHQVPFELPLRSTRISLSGNALGDEGVAQLAQLLPGLGALQSLNFESQHVVLRGDRRGSLRQCQLEPLNLTRLCETLEKCPGPLEVELSFEVLSDQSLETLLQHLPRLPQLSLLQLSQTRLSPKSLLLLADLFSLCPRVQKVDIRSLYHMTLHFRSGEEHEGECCGFTGCGLSQEHVEPLCWLLSKCEDLNQLDLSHNSISLESALGLVKTLPSCPHIREASVKAREAGPPTSPHPCLCSLGSEQSFWIYFSRKEEAGKTLRLSQCNLMPEHVPRLATHLNQALKLTALTLTQCGLGLEQLIVLLSLLRCPVDLFTLRVEEPWVGKAGVLALLRVCTQTSGNITEISISETQQQLCLQLEFPPQENPEAVTLRLAHCDLETHHSLLAGQLMETCARLQQLSLSQVNLCDSSSLLLKNLLVSLSELKTFRLTSSCMSSKGLAHLTFGLRNCHHLEELDLSNNQFGEEDTEVLMTVLEGKCWLKKLDLSHLPLGSSTLAMFTQGLSHMTLLQSLRLSRNGICDVGCHQLSEALRAAMSLKELGLGYNVLGDTTALGLAQGLPCCLRVLHLPSSRLGPKGALSLSQALDGCPHVEEISLVSCEIDNHTAKPLAASLVLCPALEEILLSWNLLGDEAAAELARVLPQMGRLKRMDLEKNRITACGAWLLAEGLAQGSGIQVIRLWNNPISPTMVQHLQSQEPRLDFAFFDK

SEQ ID NO: 3 - protein, 1868 aa, Bos taurus
MDPVSLQLGTKNLHGYLVRLLSRNLGWLDAKVKLFLPSVDLGSGYQALDPTQRVILQLQKLHAQGPTTWKSFIQCVCMELDVPMDMEVLLMSTWGHEDGFSSLLEIDEESQSDSQLHPGLKRTHQSCGSSPRPKQFRKQQQELAKRYLQLLRTSAQQRYGNRTPGPGQPLAFHQAYVPPILKWSRASAPFHTQEGATTGNPKAEDGTDVNVRDLFSTKASKGPRVTVLLGKAGMGKTTLAHRLCQKWADGQLDRFQAVFLFEFRQLNLITGSLTLPQLLFDMYLRPEASPEAVFQHLKENAEGILLIFDGLDEILQPCSDKETEDPEDPASALTLFSRLCRGTLLPGCSVMATSRPGKLPACLPTEAATVHMWGFDGPRVEEYVNHFFSDQPSQEAALAELQMDRHLWSMCAVPALCRVACLCLHHLLPGRSRGQSAALLPTMTQNYMQMLLTLNPQGVLRAEPLLGLGEVALSGLESGKVIFSAGDIPASTLDFGAALGLLTSFCVCLGPGHRDTGYAFSHLSLQEFFAALHLLASPQVDREALSQHVTLHSRWVQRTKARLGLSDHLPTFLAGLASRACRSFLSLLAEREEEWVAAKQEVVLQALKKLATRRLTGPKVVELCHRVGETQDPELASFMARSLPQQLPFHNFLLTYADLASLTSILGHRDGPIHLDFEGCPLEPRCPETLAGCGQVENLSFKSRKCGDAFAEALSKSLPTMGSLKKLGLAGSKITAQGISHLVQALPFCPQLEEVSFQDNQLKDREVLNIVEVLSCLPRLQKLDLSRNNVSVSTLLSLTKVAVTCPTIRMLQVRETDLIFLLSPPTETDPELQGDPDLQGNANQRKEAQRRSLALRLQKCQLGVHDVEVLIAQLREGPQLDEVDLSGNQLKDKGCQLMAEAAPQLHITKKLDLSDNELSVAGVLGVLSVVNTYQTLTELHISLLHKTVVFTFAPELEEQEGIQKRATFPDSLMFQMSSEPSLRSPRIRLTHCALQAQRLELLCKALGGSCRLSHLDLSGNALGDEGVALLARLLPGLGSLQSLNLSENHLSLEGVCSLTQCFSTLRWLLHLEFSSASQHVILRGDKRGRDLLAAGSLPEFRAGARLGSGQRHVPRSFCLKECQLEPLSLSYLCKSLEKCPGPLEVQLSCEVLSDQSLETLLYHLPQLPQLSLLQLSQMQLPPESPLLLADLFSLCPQVQKVDLRSLRHLTLHLRSSEEQEDRCCWRFTDCGLRQEHVEPLCGLLSKCEDLHQLDLSANLLGDDGLRCLLECLPKVPISGSLDLSHNSMSQESVLCLVEALPSCPRLREASVNLGSKQSFWIHFSRQEETGKTLRLSECHFKPEHMPRLAASLSQALQLTELTLTWCFPGLEQLTVLLGLLTWPTGVLTLRVEEPWVGRVGVLTLLEVCAQASGNITGISISETQKQLCIQLEFPHQENPEAVALRLAHCDLGTQQNLLARKLMETCARLRQLSLSQVNLCDASSLLLQSLLLSLSELKNFRLTSSCVSSKGLALLTSGLSHCHLLEELDLSNNQLGDEGTKVLLGALEGKCRLKRLDLSHLPLGGSTLAVLTQGLGHMTLLQSLRLSKSNIDDVGCCHLSKALRAATSLEELSLSHNQIGNIGAQDLAAVLPGLPELRKIDLSANGIGPAGGARLAESLTLCRHLEELMLGCNALGDCTALGLAKGLPQHLRVLHLPSSHLSPEGVLSLGQALDGCPHVEEISLAENSLAGGVPHFHQGFPVLRKLDLVSCEIDNQTAKPLAASFVLCLALEEITLSWNLLGDEAAAELARVLPQMGRLKRMDLEKNQITAYGAWLLAEGLAQGSGIQVIRLWNNRIPPDMAQHLQNQEPRLDFAFFDNQPQVIPRGT

SEQ ID NO: 4 - protein, 1840 aa, Equus
MDPDSLQLGTKSLWSWLVRLLSQAPEWMNAKVKFFLPNMDLGSRSEALDPSQRIIQQLRELYAQGPAPWQTFIHCVCMELEVPLELEVPLLSTWGHGDGFPSPLEAGEDCGPESQLQHGIKRPHQSCGSSPRPKQSRKQQLELAKRYLKLLRSSAQQRYGGRILGPGQPLVFPQAYVPPVLQWSRATAPFSTQEGALMGDPKAEDGTDVSILDLLNTRADKGPRVRVLLGKAGMGKTTLAHWLCQKWADGQLDRFQALFLFEFRQLNLITSVLTLPQLLFDLYLSPEEGPDAVFEYLEDNANGVLLIFDGLDEALQPCSSGETVDPEALGPALTLFSDLCRGTLLPGCWVMATSRPGKLPACVPTEAATVHMWGFDRPRVEEFVGRFFSDLPSQKAALAELRANRRLRSLCAVPALCRVTCVCLHHLLPGSSPGQSAALLPTMTQTYVQMMLALRPRGYLPANSLMGLCEVALSGLETGKVIFSARDIPPATLAFGAAHGLLISFCICISPGRQETGYAFPYLSLQEFFAALHLMASPEVDQDALARHVTLNSRWVLRTKARLGLSDHLPAFLSGLASCACRHFLSQLAQRDEVWVSAKQAAVAQALRKLATRRLTGPKVVELCHCVSETQESELAGLVAQSLPYQLQFHNFPLTHADLAAVTNILGHRDAPVHLNFEGCPLEPSCPEALASCGQVESLSFKSRKCGDAFAEALSRSLPTMGSLKKLGLAGSKITARGISHVVQALSLCPQLEEVSFQDNQLKDQEVLSIVKILPRLPHLRKLDLSRNHVSDWTLLHLTEVAVTCPTVRMLQVTKADLIFLLSPPAETAAELPGAPDLQESASSRKEAQSRSLTLRLQECQLGVHEVEMLLAQLRKGPHLDEVDLSGNQLEDEGCRLMAEAAPQLHITRKLDLSDNGLSMAGLYRVLGAASTCRTLAELHISLLHKTVVLTFAPEPEEQAGMQRRAVFPDSHVPQTPSELPLRSQRIRLAHCGLQAKHLELLCKALEGSHHLGHLDLSSNALGDEGAAQLAQLLPGLGPLQSLNLSENGLSPDAGFSLALCFSTVQWVLHLDITGESQRIVLGGAGRGRDLSAAGSVPELPAGAEFLGSGQRCVPRSFCLSECQLEPLSLTRLCDTLEKCPGPLEVTLSCKGLSDQSLETLLHRLPRLPQLSRLRLSQTALSPRSPLILADVFSLCPRVQKVDLQSLCHTTLHFRSSEEQEGGCCGLSANQLGDAGLQCLLEHLPRVPISGSLDLSHNRLSQESALCLVETLPSCPRIREASVNLGLRAELLGFTSPHRRRLEKTLRLTECHFRPEHVPRLASGLSQAPQLAELTLTQCCLGLEQLTVLLSLVRWPRGLLSLRVEEPWVGRAGVLTLPEVCAQASGNVTKISISETQQQLCVQLEFPRQENPGAVPLRLAHCDLGTHRSLLVRQLMETCARLRQLSLSQVNLCDASSLLLENLLLSVSELKTFRLISSRVSSEGLAHLTSGLSHCPHLEELELSNTQFGEEDTKVLMGALEGKRRLKRLNLSHLPLGGSSLAVLTQGLSRMILLQSLRLSSNGISDIGCCHLSKALRAVTSLEELGLSHNQIGDTGAQHLAAVLPGLPELRKIDLSANGIGPAGGMRLVESLALCTHLEELMLGCNALGDPTALRLAQALPPHLRVLHLPSSRLGPEGALSLARALDRCPQVEVISLAENSLAGGVPHFCQGLPLLRQIDLVSCEIDDQTAKPLAAGFMLCPALEEILLSWNLLGDEAAAELARVLPRMGRLKRVDLEKNRITACGAWLLAEGLAQGSGIQVIRLWNNLIPTDMAQRLQSQEPRLDFAFFHNQPPAPRGT

SEQ ID NO: 5 - protein, 1866 aa, Homo Sapiens
MDPVGLQLGNKNLWSCLVRLLTKDPEWLNAKMKFFLPNTDLDSRNETLDPEQRVILQLNKLHVQGSDTWQSFIHCVCMQLEVPLDLEVLLLSTFGYDDGFTSQLGAEGKSQPESQLHHGLKRPHQSCGSSPRRKQCKKQQLELAKKYLQLLRTSAQQRYRSQIPGSGQPHAFHQVYVPPILRRATASLDTPEGAIMGDVKVEDGADVSISDLFNTRVNKGPRVTVLLGKAGMGKTTLAHRLCQKWAEGHLNCFQALFLFEFRQLNLITRFLTPSELLFDLYLSPESDHDTVFQYLEKNADQVLLIFDGLDEALQPMGPDGPGPVLTLFSHLCNGTLLPGCRVMATSRPGKLPACLPAEAAMVHMLGFDGPRVEEYVNHFFSAQPSREGALVELQTNGRLRSLCAVPALCQVACLCLHHLLPDHAPGQSVALLPNMTQLYMQMVLALSPPGHLPTSSLLDLGEVALRGLETGKVIFYAKDIAPPLIAFGATHSLLTSFCVCTGPGHQQTGYAFTHLSLQEFLAALHLMASPKVNKDTLTQYVTLHSRWVQRTKARLGLSDHLPTFLAGLASCTCRPFLSHLAQGNEDCVGAKQAAVVQVLKKLATRKLTGPKVVELCHCVDETQEPELASLTAQSLPYQLPFHNFPLTCTDLATLTNILEHREAPIHLDFDGCPLEPHCPEALVGCGQIENLSFKSRKCGDAFAEALSRSLPTMGRLQMLGLAGSKITARGISHLVKALPLCPQLKEVSFRDNQLSDQVVLNIVEVLPHLPRLRKLDLSSNSICVSTLLCLARVAVTCPTVRMLQAREADLIFLLSPPTETTAELQRAPDLQESDGQRKGAQSRSLTLRLQKCQLQVHDAEALIALLQEGPHLEEVDLSGNQLEDEGCRLMAEAASQLHIARKLDLSDNGLSVAGVHCVLRAVSACWTLAELHISLQHKTVIFMFAQEPEEQKGPQERAAFLDSLMLQMPSELPLSSRRMRLTHCGLQEKHLEQLCKALGGSCHLGHLHLDFSGNALGDEGAARLAQLLPGLGALQSLNLSENGLSLDAVLGLVRCFSTLQWLFRLDISFESQHILLRGDKTSRDMWATGSLPDFPAAAKFLGFRQRCIPRSLCLSECPLEPPSLTRLCATLKDCPGPLELQLSCEFLSDQSLETLLDCLPQLPQLSLLQLSQTGLSPKSPFLLANTLSLCPRVKKVDLRSLHHATLHFRSNEEEEGVCCGRFTGCSLSQEHVESLCWLLSKCKDLSQVDLSANLLGDSGLRCLLECLPQVPISGLLDLSHNSISQESALYLLETLPSCPRVREASVNLGSEQSFRIHFSREDQAGKTLRLSECSFRPEHVSRLATGLSKSLQLTELTLTQCCLGQKQLAILLSLVGRPAGLFSLRVQEPWADRARVLSLLEVCAQASGSVTEISISETQQQLCVQLEFPRQEENPEAVALRLAHCDLGAHHSLLVGQLMETCARLQQLSLSQVNLCEDDDASSLLLQSLLLSLSELKTFRLTSSCVSTEGLAHLASGLGHCHHLEELDLSNNQFDEEGTKALMRALEGKWMLKRLDLSHLLLNSSTLALLTHRLSQMTCLQSLRLNRNSIGDVGCCHLSEALRAATSLEELDLSHNQIGDAGVQHLATILPGLPELRKIDLSGNSISSAGGVQLAESLVLCRRLEELMLGCNALGDPTALGLAQELPQHLRVLHLPFSHLGPGGALSLAQALDGSPHLEEISLAENNLAGGVLRFCMELPLLRQIDLVSCKIDNQTAKLLTSSFTSCPALEVILLSWNLLGDEAAAELAQVLPQMGRLKRVDLEKNQITALGAWLLAEGLAQGSSIQVIRLWNNPIPCDMAQHLKSQEPRLDFAFFDNQPQAPWGT

SEQ ID NO: 6 - protein, 1748 aa, Pullus
MQPTLQNDDFNLEGAVASIRPQLVEFLSHRLDWLLSASQHFLPTAVLSGLAGVTDHREKVSVLLDLLEKAGHATWKQFAQCLCMECDLPLEMEILLMSSAGEGNLTQKQEAQTDASDQSSVPRGLTRRRRSSSPISDKGAKQKRLDSAEKYQRLLVDSICKRYGSRRAAGAAAQEPTQPLAFSQAFVNLVLRQSKASRLKERTDKPREDVPSAPEPEECVDTAVRVSDLFGSVVRSGTTKVIFLFGKPGTGKTMLMHRICQKWAEGVLHQFLFTFLFEFRQLNLLKRKLTLKELLFDLFLQPEDSPDAVFQHLLENAQRTLIIFDGLDEFVGNMDVSSASKGSLNRSSSMSISELFAELCHGNLLPGCTVLVTSRPKRVPDFLINTVDLLAEVWGFDHEKVEEYVSHYFRHHSFKEQAIAHLKNNTKLLSMCQIPALCYVVCICLEYLLLKHQTSVELPQTMTQFYIKMLLIFINKQQGEHAGDEEAQLNSNKKAILGLCDLALKGLEAKKLVFYVGDIPEHVKEFASLHGLLTVFEVKTNSAHPETGYAFVHLSLQEFFAALCLMISKSVDKSHLKRKLSLKSKWTLRNEAKTGFMESFHIFLSGLSSKECRTFIMLLAEQSEAWVQDKQDAILQSLKKLAATQLTGPKVIELCHCTFETQGLEVAQHIGSLLNFKYEFKNFRLTPLDMSALVFVINSGQDVTHLDFAGCPLDTDCLEMLASCRNVEHLSFRSRRFGDDFAAALSKGLGEMGSLKKLEVTGGSITAAGLTDMVQALSHCLQLEEINLQDNRIQNPDVKTVMELFSRMEKLKKIDLSKNSLSLNAVLLLAKEFIACQNAAELHVRKDTVIISFSGPSGKVPRSLDLKREQNKECVTPTRHLRLCLQARCLSSQHAKEIVSILQSCPHLSEVDLSDNKLGDEGCSFLLENLSWISISKQLNLYHNTAVLRFTEDGEFASPPASREEPLYPTDDQWDDKENQTPVPSKKIRLTYCRFQASDLEKLCAVLKECGSISELELNNNHLSLSSVFCLAQSLCTLEHLETMDLSLGRMQVVHLTFGDRIRLRRTSRWRSCFLVHPKHVTNGQCFRLRNCTVGPEDVTRLCQMLMQCTQLTEIDLCGNALNDQSIERLLSFLPHFCQLTLLSIRNNTFSPCCAVLFANSINLCERIRRVEVRSSQNAFLHLRTSTQGQKTSCRLTDCAIGQRQIEKLCRVLEQHGCLAELDLSRNQLGDEVLRFLLDHLHRVPVTCSLNLSHNRISQRGVLHLINTFTTSGNTTEVQVSLCSKATLIIKMTSRDDPRKILRLTECSFQPEHLEKLCLVLENCTNLTECISSNNNVTVDAAGGLLRSLSKTPGPLKISIEEPWVCKLSVTSLLELAVQAHGNITAIMICKEKNLFQLGVRLPHCLEKVGSVVSRLNLNEPEIKQACFYQRVHDKCTQLQELRWSHVELHDDTEMLVSILLPLPDLKKFELTSCSFTPTGIDCLITGLQRCQAIEELNLGHMKLGDAAIPKLVFGLCEMPSLKRLILNHNSIGDDGCSRLAEALSSMHCMEEINLGHNKIGDLGLINIAAVLLEMQNLKRIDLSGNCPSPAGGEKLMEALANCKHLEELILSENNLGEGGIRALSEGLPRFEHLRKIELRLCGITDDASKLLSRGFQQCPAVEEIILSWNALGDGGAQVLASILPGMETLKMLDLEKNLIGACGATRLAEELVRCPEIQFIRLWDNPVPKGLAESLTSQDPRLCFSFC

SEQ ID NO: 7 - DNA, 5235 bp, Canis familiaris
atggcttttaccttcctgaaggtgctcaacaacatggagattgacacttctctgtttgatgaagtgggagccaagatcatcaaagacctgatgtccaaagctgagaagaatggtgtgaaaattatcttgccagttgactttgtcactgctgaaaagtttgatgagcatgccaggactggccaagccactgtggcctctggcatacctgctggctggatgggcttggactgtggtcctgagagcagcaagaagtatgctgaggcagttgcttgggctaagcagatcatgtggaatggacctgtgggcgtatctgaatgggaagctcttgcccgaggaaccaaagccctcatggatgaggtggtgaaagccactgcctgggactgcatcaccatcataggtggtggagacattgccatttgctgtgtcaaatggaatacagaggataaagtcagccatggccagctcctcatggaccacgttagcttccagctgggtaccactaacctgtggagctggctcgtgaggctgctcagtaaagacccagaatggctgaatgccaaggtgaagttcttcctccccaagatggacctgagctccggggacgaggcccaggacaccacacagagggtcatgctacagctcagaagattgcacacccaaggtcgggccacttggcaggcattcatccactgtgtgtgcatggagctcgatgtgcccctggacctggaggtgccactgctgagcacttggggccatggagatggtctgaagcggcctcatcagagctgtgggtcctcaccccgcctgaagcagtgcaagaatcatcagctcgagttggccaggaggtacctgaagttgctgaggacttctgcccagcagcgctacagcaggattcccgggccggggcaaccccttgccttccaccaggcctacatccccccaatcctgcagtggagccggaccacggcccccttcagtgctcaggacggggctgtagtgggggaccccaaggcagaagatgacacggacgtgagcatccgggacctcttcagcaccagggccgacaagggcccgagggtgacggtgctcctggggaaggcaggcatgggcaagaccacgctggcccaccggctctgccagaggtgggctgctggccagcttgaccgcttccaggctctgttcctgtttgaattccgtcagctcaatctgatcaccagttccgtgacgctgccccagctgctgtttgacctgtacctgagccccgaggcgggccccgactcggtcttccagtacctggaggagcatgctggaggagtcctgctgatctttgatgggctggaccaggccctccactcgcattccagcacggagcctgaaggccccgaggccccaagtccggcccttgccctcttctccgggctctgccgggggaccatcctgcctggctgctgggttttggccacctcccgtccagggaagctgcccgactgcctgcccacagaggcagccacggtccacatgtggggctttgacaggccacgggtggaggagtatgtgggccgcttcttcagtgaccaagagtggcaggaagcagccctagcagagctgcgagaaaacaggcatctccagagcatgtgtgcggtgcctgcactgtgccaagtcacctgcctctgcctccaccatctgctcccaggcaactctccaggccagtctgcagccctcctgcccaccgtgactcagaactatgtacagatggtgtccactctcagcccccgtgggtacctgcctcccgagtctctgctgggcctcagtgagatggctctgaggggcctggagactgggaaggttatcttctctgtaggcgacatctctccacccatggtggcttttggggcagccctcggcttgttgacctccttctgcatctgcacaggccctgggcaccagggtataggctatgccttcacccacctcagcctacaagagttttttgccgccttgtacctgatggccagccccaaggtggacaaaaatgtacttgcccgccatgtcaccctcaattctcgctgggtgctacggaccaaagctagactaggcctcttagaccacctccccaccttcctggcagggctggcatcctgtgcctgccgacccttccttagccatgtggcccagcaggatgagatgtgggtgagtgacaagcaggctgcagtgatgctggctttgaggaagttggccattcgcaggctcacaggacccaaggttgtagcactctgtcactgtgtgggtgagacccaggagcctgagctggccagcctcatggcccaaagtctccccagtcacctgtccttccacaatttcccactgacctatacggacctgtctgccctgaccaacatcttgaggcacaggaatgcctccatccacctggattttgagggctgccccctggagccccactgccccgaggccctggcaggctgtaagcagatagagaatctcagctttaagagcaggaaatgtggggatgcctttgcagaagccctctccaagagcctgccaacaatggagagcctgaagaagctcgggttagcaggatgtaagatcactgcccgaggcatcagccacctgatgcaggctttgcgcctctgtccacagctggaagagatcagcttccaggacaaccagctgaaggatagggccgtgctgaatatcgtggaagtacttccttctttgccacggctccagaagcttgacctgagccgcagcaatgtctccgtgtcaaccttactctgcttgacaaaggtggcggtcacatgccctactgtcaagatgctacaggtcaggctcctgcagtgccagctcggaattcatgatgtgcaggagctcattgcccagctccgggaaggtccccgcctggaggaagtaaacctcagcaacaacgggctatctgtggacagggtacactgtatgctgagtgcggtgaacacatgccagaacttggcagagcttcatatcagcctgctgcataaaactgtggtcttcacatttgccccagagcaggaggagcaggaggggatctggaagaggactgcgcttcgggacagcctcacgcaccaggtgccctttgagctgcccctgcgctccacgaggatcagcctctcaggcaatgctctgggggatgaaggcgtggcccagctggctcagctgctcccagggctgggtgctctgcagtccttgaacttcgagagccagcacgtcgtcctgagaggtgacagaagaggcagcctcaggcaatgtcagctggagcccctgaacctcacccgcctctgcgagactctggagaagtgccccgggcctctggaagtcgaattgtcctttgaggtcctgagcgaccagagcctggagaccctgctgcagcacttaccccggctgccccagctaagcctgctgcagctaagccagacgaggctttccccaaagagcctcctcctgctggccgacctcttcagcttatgcccccgggttcagaaggtggatatcaggtccctgtatcacatgacactgcacttcaggtctggcgaggagcatgaaggcgagtgctgtgggttcacgggctgtggccttagtcaggagcatgtggagccgctctgctggttgctgagcaaatgcgaagacctcaaccagttggacctgagtcacaacagcatctccctagagagtgccctgggcctggtgaagacactcccctcctgcccgcacatccgggaggcttctgtgaaggctcgggaagctgggcctccgacctccccacacccttgtctctgcagcctgggctctgagcagagcttctggatctacttctcccgaaaggaggaggccgggaagacactaaggctgagccagtgcaacctcatgccagagcatgtgcccagactggccacccacctgaaccaggccctgaagctgacagcgctcacgctgacccagtgcggcctgggcctggagcagctgatagttctcctgagtctgctgagatgtccagtggacctgttcactctcagggtggaggagccgtgggtaggcaaagccggggtgctcgccctgctaagagtctgcacccagacctcaggcaacatcactgaaataagcatttcggagacccagcagcagctctgtctccagctggagtttccccctcaggagaacccagaagccgtgacgctcaggttggctcactgtgaccttgagacccaccacagccttcttgctgggcagctgatggagacatgtgccaggctgcagcagctcagcttgtcccaggtgaacctctgcgactccagctccctgctgctaaaaaacctcctggtgtctctctctgagctgaagacattcagactgacctccagctgtatgagctccaagggcctggcccacctgacatttggcctgagaaactgccaccacctggaggagctggacttgtccaacaatcagtttggtgaggaggacactgaagtcctgatgacggtccttgagggcaagtgctggctgaagaagctcgacctcagccatctcccactaggcagctccaccctggccatgttcactcaaggactaagccacatgaccctcctacagagcctccggctgagcaggaacggtatttgtgatgtcggctgccaccaactttccgaggccctcagagctgccatgagcttgaaggagctggggcttggctacaacgtcctgggggataccacagccctgggcctggcccagggactgccctgctgcctgagggtcctgcacctgccgtctagccgtctgggcccaaagggagcgctgagcctgagccaggccctggatggatgcccacatgtggaagagatcagcctggtttcgtgtgagatcgacaaccacaccgccaagcccctggctgccagcctggtgctctgcccagccctggaagaaatcctgctgtcctggaatctgctcggggacgaggcggctgccgagttggcccgggtcctgccgcagatgggccggctgaagagaatggacctggagaagaatcggatcacagcttgtggagcctggctcctggctgaagggctggcacaagggtctggcatccaagtcattcgcctctggaacaatcccatctcccccaccatggtccagcatctacagagccaggagcccaggctggacttcgctttcttcgacaaatag

SEQ ID NO: 8 - DNA, 5932 bp, Bos taurus
atggaccccgttagcctccaactgggcaccaagaacctgcatggatatctcgtgaggctgctcagcagaaacctaggatggctagacgccaaggtgaagttattcctccccagcgtggacctgggctccggatatcaggccctagaccccacacagagggtcatcctgcaactccaaaaactgcatgcccagggtccgaccacctggaagtcattcatccagtgcgtgtgcatggagctggatgtgccaatggacatggaggtgctgctgatgagcacgtggggccacgaagatggcttctccagtcttctggaaattgacgaggaaagccaatcagactctcagctccaccctggcctcaagcggactcatcagagctgtgggtcctcacctcgcccaaaacagttcaggaagcagcaacaagagttggccaagaggtacctgcagctactgagaacttctgcccagcagcgctatggcaacaggacccccgggccgggacagccattggccttccaccaagcctacgtccctccaatcttgaaatggagccgggcctcggcgcccttccacactcaggagggtgccacaacagggaaccccaaggcagaagacggcactgatgtgaacgttcgggacctcttcagtaccaaggccagcaagggcccgagagtgacggtgcttctggggaaggcgggcatgggcaagaccacgctggctcaccggctctgccagaagtgggccgatggtcagctggaccgcttccaggctgtgttccttttcgaattccgccagctcaacctgatcacaggttccttgacactgccgcagctccttttcgacatgtacctaaggcctgaggccagcccggaggccgtcttccagcacctgaaggagaacgcggagggaatcctgctgatcttcgacgggctggacgagatcctccagccctgctctgacaaggagaccgaggaccccgaggacccagcgtcagccctcaccctcttctcccgcctctgccgtgggaccctgctgcccggctgctcggtgatggccacctcccgtccagggaagctgcccgcctgcctgcccaccgaggcggccacggtccacatgtggggcttcgacggcccgcgggtggaggagtatgtgaaccacttcttcagcgaccagccgtcccaggaggcggccctggcggagctgcagatggacagacacctctggagcatgtgtgcagtgcctgcgctgtgccgagtcgcctgcctctgcctccatcacctgctcccgggcaggtctcgaggccagtctgcagccctcctgcccaccatgacgcagaactacatgcagatgctcctcaccctcaacccccaaggggtcctgcgggctgagcccctgctgggtctcggggaggtggccctgagcggcctggagtcagggaaggtcatcttctctgcaggagacatccctgcatccaccctagactttggggctgccctcggtctgctgacctccttctgcgtgtgcctggggcccgggcaccgggacacaggctacgccttctcccacctcagcctgcaggagttcttcgcagccctgcacctgctggccagcccccaggtggacagagaggccctcagccaacatgtcaccctccattctcgctgggtgcagcggacaaaagccagactgggcctgtcggaccacctgcccacctttctggctggcctggcatcccgtgcctgccgctccttcctcagcctcctggctgagcgggaggaggaatgggtggctgccaagcaggaggtagtcctgcaggcgctgaagaagttggccacccgtcggctcacggggcccaaggttgtagagctgtgtcaccgtgtgggtgagacgcaggatcctgagttggccagcttcatggcccgcagcctcccccagcagctgcccttccacaacttcctgctgacctacgccgacctggcctccctgaccagcatcctggggcacagggatggccccatccacctggattttgagggctgccccctggagccccgctgccctgaaaccctggcaggctgtgggcaggtagagaatctcagctttaagagtcggaagtgtggggacgcctttgcagaagccctctccaagagcttgccaacgatggggagcctgaagaagctggggttagcaggaagtaagatcactgctcaaggcatcagccacctggtgcaggctttgcccttctgcccacagctggaggaggtcagcttccaggacaatcagctcaaggacagggaagtgctgaacatcgtggaggtactctcttgcctaccacggctccagaaacttgacctgagccgcaacaatgtctccgtgtcaacactactctccttgacaaaggtggcggtcacgtgccctaccatcaggatgctgcaggtcagggagacggacctcatcttcctcctctccccgcccacagagacagatcctgagctacaaggagacccagacttacagggaaatgccaaccagaggaaagaggctcagagaagaagcctggcactcaggctccagaagtgtcagcttggggtccatgatgtggaggtgctcatagcccagctgcgggaaggccctcaactggatgaagtggacctctcagggaaccagctcaaagataaaggctgtcaactgatggcagaggctgctccccaactgcacatcacgaagaagctggacctcagcgacaatgagctttctgtggccggggtgctgggagtactgagtgtggtgaacacgtaccaaaccctgacagagctacatatcagcctgctgcacaaaactgtggtcttcacgtttgccccagagctggaggagcaggaggggatccagaagagggctacgtttcctgacagccttatgttccagatgtcctctgagccatctctgcgctccccgaggatcaggctgacgcactgtgccctgcaagcccagcgcctggagctgctctgcaaagcgctgggaggaagttgccgcctcagtcaccttgacttatcaggcaatgctctgggggatgaaggtgtggccctgctggctcggctgctccccggactgggttctctgcagtccttgaacctcagtgagaaccatttgtccctggagggtgtgtgcagtttgacccagtgcttctctactctgcggtggcttctacatttggaattcagctctgcaagccaacacgtcatcctgagaggcgacaaaagaggcagggatctgttggctgctggatctttgccagagttccgagctggagcccggttgggatctggtcagcgccacgtccccaggagcttctgcctcaaggaatgtcagctggagcccctgagcctctcctacctctgcaagtctctggagaagtgcccagggcccctggaagtccaattgtcctgcgaggtgctgagtgaccagagcctggagaccctgctgtaccacctaccgcagctcccccagctaagcctgctgcaactgagccagatgcaactgcctccagagagccccctcctgctagctgacctcttcagcctgtgcccacaggttcagaaggtggatctcaggtccctgcgtcatttgaccctgcatctcaggtctagtgaggagcaggaagacaggtgctgttggaggttcactgactgcggcctccgccaggagcacgtggagccgctgtgcgggttgctgagcaagtgtgaagacctccaccagctggacctctcagcaaacctgctgggtgatgatgggctcaggtgcctcctggaatgtctccccaaggtgcccatttccggttcgcttgatctaagccacaacagtatgtctcaggaaagcgtcttgtgcctggtggaggcgctcccctcctgcccacgtctccgggaggcctcagtgaacctgggctccaagcagagcttctggattcacttctcccgacaggaagagactgggaagacactgaggctgagcgagtgccacttcaagccagagcacatgcccagactggccgccagcctgagccaggccctgcagctgacggagctcacgctgacctggtgtttcccgggcctggagcagctgactgtcctcctgggtctgctaacgtggccgacgggggtgctcactctcagggtggaagagccatgggtgggcagagtcggggtgctcacgctgctggaagtctgtgcccaggcctcaggcaacatcactggaataagcatctccgagacccagaaacagctctgtatccagctggaatttccccatcaggagaacccagaagccgtggccctcaggctggctcactgtgacctgggaacccaacaaaacctcctcgccaggaagttgatggagacatgtgcccggctgcggcagctcagtttgtcccaggtgaacctctgcgatgccagctctctgctgctgcaaagcctcctgctgtccctctctgagctgaagaacttcaggctgacctccagctgtgtgagctccaaggggctagccctcctgacatctggtctgagccattgtcaccttctggaggagctagacttgtctaacaatcaacttggagacgagggcaccaaggtgttgttgggggctcttgagggcaagtgcaggctgaagagacttgacctcagccacctgccactgggcggctccaccctggctgtgctcactcaaggactaggccacatgacactcctgcagagcctccgtctgagcaagagcaacatcgatgacgtcggctgctgccacctttcgaaggctctcagagctgccaccagcttggaggagctgagcttgagccacaaccagatcggaaacatcggtgcccaggacttagctgctgtcctgccggggctccctgagctcaggaagatagacctctcagcaaatggcatcggcccagcaggcggagcgcggttggcagaatctctcaccctttgcaggcacctcgaggagctgatgcttggctgcaatgccctgggagactgcacagccctgggtctggccaaggggctgccccagcacctgagggtcctgcacctgccatccagccatctgagcccagagggggtgctgagtctgggccaggccctggatggatgcccacacgtggaagagatcagcttggcagagaacagcctggctggaggggtcccgcatttccatcaggggttcccagtgctccggaagcttgacctggtgtcgtgtgagattgacaaccagaccgccaagcccctcgccgccagcttcgtgctctgtctggccctggaagaaatcacgctgtcctggaatctgctcggggatgaggcagctgctgagctggcccgggtcctgccgcagatgggccggctgaagagaatggacctggagaaaaatcagatcacagcttatggagcctggctcctggctgaagggctggcacagggatctggcatccaagtcattcgcctctggaataaccgtattcccccggacatggcccagcatctgcagaaccaggagcccaggctggactttgctttctttgacaaccagccacaggtcatccctcggggtacctgatggtgccctcaagtccctttgaatccagccgagtgatgccagttttcatccatgaggatagagggctcatgaaaaaagctggctgtccctggatcaagagggagaaacacacttttcctgcaacagtcttcagggaggacatttttggtgacaaagagcggagtatgactaccctacatcccttgggacatgcatgaccagtcaggaacatgaagcaaaatgagcgtgtcatggtgtggtgtgtgatgtggaaggtcacacgtggcgacattccatgtgaccttgcacgatacctgaaacgtggcctgggggtcgatggcatgcg

SEQ ID NO: 9 - DNA, 5693 bp, Equus
cagtttcaggtggcgcgcggcggcgcggggaccgcggctggggcctccccactggaaacaacctgcaaatccagctcaggccgtggggcttggccgaggcacccatgagaccctctccgcagacgcgctagaacacctatgatgaaccaggaggccaaggcaagctcctcatggaccccgatagcctccagctgggcaccaagagcctgtggagctggctcgtgaggctgctcagccaagccccagaatggatgaacgccaaggtgaagttcttcctccccaacatggacctgggctccaggagcgaggccctggacccctcgcagaggatcatccaacagctcagggaactgtacgcccagggcccggccccctggcagacgttcatccactgcgtgtgcatggagctggaggtgccgctcgagctggaggtgccgctgctgagcacgtggggccacggagacgggtttcccagtccgctggaagctggtgaggactgcggaccagaatctcagctccagcacggtatcaagcggcctcatcagagctgtgggtcctcgccccgcccaaagcagtccaggaagcagcagctagagttggccaagaggtacctgaagctgctgaggagttctgcccagcagcgctacggcggcaggatcctggggccggggcagccgcttgtcttcccccaggcctacgtccccccagtcctgcagtggagccgagccacggcgcccttcagcactcaggagggggcccttatgggggaccccaaggcagaagatggcaccgacgtgagcatcctggacctcctcaacaccagggctgacaagggtcccagggtgagggtgcttctggggaaggcgggcatgggcaagaccacactagcccactggctctgccagaagtgggccgacggccagctggaccgcttccaggccctgttcctttttgaattccgccagctcaacctgatcacaagtgtcctgacactgcctcagctcctttttgatctgtacctgagccccgaggagggccctgacgccgtctttgagtacctggaggacaatgctaatggggtcctgctgatctttgatgggctggacgaggccctccagccctgttccagcggagagactgtagatcctgaggccctaggtccagccctcaccctcttctctgacctctgccgtgggaccctcctgcccggctgctgggtaatggccacctcacgtccagggaagctgcccgcctgcgtgcccacggaggcggccacggtccacatgtggggttttgacaggccacgggtggaggagttcgtgggccgcttcttcagcgacctgccgtcgcagaaggccgccctggcagagttacgggcaaacagacgtctccggagcttgtgtgcggtgcctgcactgtgccgtgtcacctgcgtctgcctccaccatctgctcccaggcagctccccaggccagtctgcggccctcctccccaccatgactcagacctatgtgcagatgatgctcgccctcaggccccgtgggtacctgcctgccaattccctgatgggtctctgtgaggtggccctgagtggcctggagaccgggaaggttatcttctctgccagagacatccctccagccacgttggcctttggggctgcccacggcctgctgatctccttctgcatctgcatcagccctgggcgccaggagacaggctatgctttcccctacctcagcctgcaggagttttttgctgccctgcacctgatggccagccccgaggtggaccaagatgcactcgcccgccatgtcaccctgaattctcgctgggtgctgcggaccaaagctcgactgggcctctcagaccaccttcccgccttcctgtcgggcctggcatcctgtgcctgccgccacttcctcagccagctggcacagagagacgaggtgtgggtgagtgccaagcaggctgcggtcgcgcaggcattgaggaagttggccacccgcaggctcacggggccgaaggtggtagagctgtgtcactgtgtgagcgagacacaggagtctgagctggccggcctggtggcccagagtctcccctatcaactgcagttccacaacttcccactgacccacgccgacctggctgctgtgaccaacatcctggggcacagggatgctcctgtccacttgaattttgagggctgccccctggagccctcctgccctgaggctctggcaagctgtgggcaggtagagagtctcagctttaagagcaggaagtgtggggatgcctttgcagaagccctctccaggagcctgccaacaatggggagcctgaagaagctggggttagcgggaagtaagatcactgcccgaggcatcagccacgtggtgcaggctttgtccctctgtccacagctggaagaggtcagctttcaggacaaccagctcaaggaccaggaggtgctgagcatcgtgaagatactccctcgcctgccgcacctccggaagcttgacctgagccgcaaccatgtctctgactggaccctactccacttgacagaggtggcagtcacgtgccctaccgtcaggatgctacaggtcacgaaggcggacctcatcttccttctctccccacctgcagagacagctgcagagctaccaggagccccagacctacaggaaagtgccagctcaaggaaagaggctcagagcagaagcctgacactcaggctacaggagtgtcagctcggggtccatgaggtggagatgctcttagcccagctccggaaaggcccccacctggacgaagtggacctttcagggaaccagctggaagatgaaggctgcagactgatggcagaggccgcgccccagctccacatcaccaggaagctggacctcagcgacaatgggctctctatggccgggctgtaccgcgtgctgggtgcagcgagcacgtgccggaccctggcagagctgcacatcagcctgctgcacaaaaccgtggtcctcacctttgccccagagccagaggagcaggcggggatgcagaggagggctgtgtttcctgacagccacgtgccccagacgccctctgagctgcccctgcgctcccaaaggatcaggctggcgcactgtggcttacaagccaagcacctcgagctgctctgcaaggcgctggaaggaagccaccacctgggccacctcgacttatcaagcaatgctctgggggacgaaggtgcagcccagctggctcagctgctcccagggctgggccctctgcagtccttgaacctcagtgagaacggcttatccccggacgctgggttcagcttggccctgtgcttctctactgtgcagtgggttctccacctggacatcacgggtgagagccaacgcatcgtcctgggaggagccggaagaggcagggacctgtcggctgccggatctgtgccggagttgccagctggagccgagttcttgggctccggtcagcgctgcgtccccaggagcttctgcctcagtgagtgtcagctggagcccctgagcctcacccgcctctgtgacactctagagaagtgcccggggcctctggaagtcacattgtcctgcaagggcctgagcgaccagagcctggagaccctgctgcaccgcttgccccggctgccccagctcagccggcttcggctgagccagacagcactgtccccaaggagccccctcatactggcagacgtcttcagcctatgcccacgggttcagaaggtggatctccagtccctgtgtcacacgaccctgcacttcaggtctagcgaggagcaggagggcggatgctgtggcctctcagcaaaccagctgggtgatgccgggctccagtgcctcctggagcatctgcctcgggtgcccatctccggttcgcttgatctcagtcacaacagactctctcaggaaagtgccctgtgcctggtggagacgctcccctcctgcccacgcatccgggaggcctcggtgaacctggggctccgagcagagcttctgggattcacttctccccaccggaggaggctggagaagaccctgaggctgaccgagtgtcacttcaggccagagcacgtgccccgactggcctccggcctgagccaggccccacagctggcggagctcacgctgacccagtgctgcctgggcctggagcagctgaccgtcctcctgagcttggtgaggtggccgagggggctgctcagtctcagggtggaggagccgtgggtgggcagagccggggtgctcacgctgccagaagtctgcgcccaggcctcaggcaacgtcactaaaataagcatctccgagacccagcagcagctctgtgtccagctggaatttccccgccaggaaaacccaggagctgtgcctctcaggttggctcactgtgaccttgggacccaccgcagcctccttgtcaggcagctgatggagacctgtgccaggctgcggcagctcagcttgtcccaggtgaacctctgcgacgccagctccctgttgctggagaacctgctgctgtccgtgtctgagctgaagacattccggctgatctccagccgtgtgagttccgagggcctggcccacctgacatctggtctgagccattgtccccacctggaggagctggaattgtccaacactcagtttggcgaggaggacaccaaggtgctgatgggggcccttgagggcaagcgccggctgaagaggctcaacctcagccacctcccgctgggcggctccagcctggcggtgctcactcaaggactcagccgaatgatccttctgcagagcctccgtctgagcagtaacggcatcagtgacatcggctgctgccacctctccaaggccctcagagctgtgaccagcttggaggagctgggcttgagccacaaccagattggagacactggtgcccagcacttagctgccgtcctgccggggctgcctgagctcaggaaaatagacctctcagcaaatggcatcggcccggctgggggaatgcggttggtggagtcgctcgccctttgcacacacctggaggagctgatgctcggctgcaatgccctgggggatcccacggccctgaggctggcccaggcgctgcccccgcacctgagggtcctgcacctgccctccagccgtctgggccccgagggggcactgagcctggcccgggccctggacagatgcccgcaggtggaagtgatcagcttggcagagaacagcctggccggaggggtcccgcacttctgccaggggctcccactgctcaggcagatagacctggtttcctgtgagattgatgaccagacggccaagccgctggctgccggcttcatgctctgcccggccctggaagaaatcttgctgtcctggaatctgctcggggatgaggcggctgctgagctggcccgtgtcctgccgcggatgggccggctgaagagagtggacttggagaagaatcggatcacagcttgtggagcctggctcctggctgaagggctggcgcaggggtccggcatccaagtcatccgcctctggaataacctcatccccactgacatggcccagcgtctgcagagccaggagcccaggctggactttgctttcttccacaaccagccaccagctcctcggggtacttga

SEQ ID NO: 10 - DNA, 6822 bp, Homo sapiens
gcagcagccgccagttccatgcactggcggagcagctctaggcggcggcttctactttcagtttcgtgcagagcgcggaggagccgcgagcgctgagggagtctgcactatggaaacaacctgtcaatccagctcaaggcacacatagcccagacacccatgagaccctctccgtggggaccctagagcacctatcatgaacgaggagaccaaggctggctcctcatggaccccgttggcctccagctcggcaacaagaacctgtggagctgtcttgtgaggctgctcaccaaagacccagaatggctgaacgccaagatgaagttcttcctccccaacacggacctggattccaggaacgagaccttggaccctgaacagagagtcatcctgcaactcaacaagctgcatgtccagggttcggacacctggcagtctttcattcattgtgtgtgcatgcagctggaggtgcctctggacctggaggtgctgctgctgagtacttttggctatgatgatgggttcaccagccagctgggagctgaggggaaaagccaacctgaatctcagctccaccatggcctgaagcgcccacatcagagctgtgggtcctcaccccgccggaagcagtgcaagaagcagcagctagagttggccaagaagtacctgcagctcctgcggacctctgcccagcagcgctacaggagccaaatccctgggtcagggcagccccacgccttccaccaggtctatgtccctccaatcctgcgccgggccacagcatccttagacactccggagggggccattatgggggacgtcaaggtggaagatggtgctgacgtgagcatctcggacctcttcaacaccagggttaacaagggcccgagggtgaccgtgcttttggggaaggctggcatgggcaagaccacgctggcccaccggctctgccagaagtgggcagagggccatctgaactgtttccaggccctgttcctttttgaattccgccagctcaacttgatcacgaggttcctgacaccgtccgagctcctttttgatctgtacctgagccctgaatcggaccacgacactgtcttccagtacctggagaagaacgctgaccaagtcctgctgatctttgatgggctagatgaggccctccagcctatgggtcctgatggcccaggcccagtcctcacccttttctcccatctctgcaatgggaccctcctgcctggctgccgggtgatggctacctcccgtccagggaagctgcctgcctgcctgcctgcagaggcagccatggtccacatgttgggctttgatgggccacgggtggaagaatatgtgaatcacttcttcagcgcccagccatcgcgggagggggccctggtggagttacagacaaatggacgtctccgaagcctgtgtgcggtgcccgcactgtgccaagtcgcctgtctctgcctccaccatctgcttcctgaccacgccccaggccagtctgtggccctcctgcccaacatgactcagctctatatgcagatggtgctcgccctcagcccccctgggcacttgcccacctcgtccctactggacctgggggaggtggccctgaggggcctggagacagggaaggttatcttctatgcaaaagatattgctccacccttgatagcttttggggccactcacagcctgctgacttccttctgcgtctgcacaggccctgggcaccagcagacaggctatgctttcacccacctcagcctgcaggagtttcttgctgccctgcacctgatggccagccccaaggtgaacaaagacacacttacccagtatgttaccctccattcccgctgggtacagcggaccaaagctagactgggcctctcagaccacctccccaccttcctggcgggcctggcatcctgcacctgccgccccttccttagccacctggcgcagggcaatgaggactgtgtgggtgccaagcaggctgctgtagtgcaggtgttgaagaagttggccacccgcaagctcacagggccaaaggttgtagagctgtgtcactgtgtggatgagacacaggagcctgagctggccagtctcaccgcacaaagcctcccctatcaactgcccttccacaatttcccactgacctgcaccgacctggccaccctgaccaacatcctagagcacagggaggcccccatccacctggattttgatggctgtcccctggagccccactgccctgaggctctggtaggctgtgggcagatagagaatctcagctttaagagcaggaagtgtggggatgcctttgcagaagccctctccaggagcttgccgacaatggggaggctgcagatgctggggttagcaggaagtaaaatcactgcccgaggcatcagccacctggtgaaagctttgcctctctgtccacagctgaaagaagtcagttttcgggacaaccagctcagtgaccaggtggtgctgaacattgtggaggttctccctcacctaccacggctccggaagcttgacctgagcagcaacagcatctgcgtgtcaaccctactctgcttggcaagggtggcagtcacgtgtcctaccgtcaggatgcttcaggccagggaggcggacctcatcttccttctttccccgcccacagagacaactgcagagctacaaagagctccagacctgcaggaaagtgacggccagaggaaaggggctcagagcagaagcttgacgctcaggctgcagaagtgtcagctccaggtccacgatgcggaggccctcatagccctgctccaggaaggccctcacctggaggaagtggacctctcagggaaccagctggaagatgaaggctgtcggctgatggcagaggctgcatcccagctgcacatcgccaggaagctggacctcagtgacaacgggctttctgtggccggggtgcattgtgtgctgagggccgtgagtgcgtgctggaccctggcagagctgcacatcagcctgcagcacaaaactgtgatcttcatgtttgcccaggagccagaggagcagaaggggccccaggagagggctgcatttcttgacagcctcatgctccagatgccctctgagctgcctctgagctcccgaaggatgaggctgacacattgtggcctccaagaaaagcacctagagcagctctgcaaggctctgggaggaagctgccacctcggtcacctccacctcgacttctcaggcaatgctctgggggatgaaggtgcagcccggctggctcagctgctcccagggctgggagctctgcagtccttgaacctcagtgagaacggtttgtccctggatgccgtgttgggtttggttcggtgcttctccactctgcagtggctcttccgcttggacatcagctttgaaagccaacacatcctcctgagaggggacaagacaagcagggatatgtgggccactggatctttgccagacttcccagctgcagccaagttcttagggttccgtcagcgctgcatccccaggagcctctgcctcagtgagtgtcctctggagcccccaagcctcacccgcctctgtgccactctgaaggactgcccgggacccctggaactgcaattgtcctgtgagttcctgagtgaccagagcctggagactctactggactgcttacctcaactccctcagctgagcctgctgcagctgagccagacgggactgtccccgaaaagccccttcctgctggccaacaccttaagcctgtgtccacgggttaaaaaggtggatctcaggtccctgcaccatgcaactttgcacttcagatccaacgaggaggaggaaggcgtgtgctgtggcaggttcacaggctgcagcctcagccaggagcacgtagagtcactctgctggttgctgagcaagtgtaaagacctcagccaggtggatctctcagcaaacctgctgggcgacagcggactcagatgccttctggaatgtctgccgcaggtgcccatctccggtttgcttgatctgagtcacaacagcatttctcaggaaagtgccctgtacctgctggagacactgccctcctgcccacgtgtccgggaggcctcagtgaacctgggctctgagcagagcttccggattcacttctccagagaggaccaggctgggaagacactcaggctaagtgagtgcagcttccggccagagcacgtgtccaggctggccaccggcttgagcaagtccctgcagctgacggagctcacgctgacccagtgctgcctgggccagaagcagctggccatcctcctgagcttggtggggcgacccgcagggctgttcagcctcagggtgcaggagccgtgggcggacagagccagggttctctccctgttagaagtctgcgcccaggcctcaggcagtgtcactgaaatcagcatctccgagacccagcagcagctctgtgtccagctggaatttcctcgccaggaagagaatccagaagctgtggcactcaggttggctcactgtgaccttggagcccaccacagccttcttgtcgggcagctgatggagacatgtgccaggctgcagcagctcagcttgtctcaggttaacctctgtgaggacgatgatgccagttccctgctgctgcagagcctcctgctgtccctctctgagctgaagacatttcggctgacctccagctgtgtgagcaccgagggcctcgcccacctggcatctggtctgggccactgccaccacttggaggagctggacttgtctaacaatcaatttgatgaggagggcaccaaggcgctgatgagggcccttgaggggaaatggatgctaaagaggctggacctcagtcaccttctgctgaacagctccaccttggccttgcttactcacagactaagccagatgacctgcctgcagagcctcagactgaacaggaacagtatcggtgatgtcggttgctgccacctttctgaggctctcagggctgccaccagcctagaggagctggacttgagccacaaccagattggagacgctggtgtccagcacttagctaccatcctgcctgggctgccagagctcaggaagatagacctctcagggaatagcatcagctcagccgggggagtgcagttggcagagtctctcgttctttgcaggcgcctggaggagttgatgcttggctgcaatgccctgggggatcccacagccctggggctggctcaggagctgccccagcacctgagggtcctacacctaccattcagccatctgggcccaggtggggccctgagcctggcccaggccctggatggatccccccatttggaagagatcagcttggcggaaaacaacctggctggaggggtcctgcgtttctgtatggagctcccgctgctcagacagatagacctggtttcctgtaagattgacaaccagactgccaagctcctcacctccagcttcacgagctgccctgccctggaagtaatcttgctgtcctggaatctcctcggggatgaggcagctgccgagctggcccaggtgctgccgcagatgggccggctgaagagagtggacctggagaagaatcagatcacagctttgggggcctggctcctggctgaaggactggcccaggggtctagcatccaagtcatccgcctctggaataaccccattccctgcgacatggcccagcacctgaagagccaggagcccaggctggactttgccttctttgacaaccagccccaggccccttggggtacttgatggccccctcaagacctttggaatccagccaagtgatgcacccaaatgatccacctttcgcccactgggataattgactcaggaaagaagagcctcggcagggcgctctgcactccacccaggaggaaggatacgtgtgtcctgctgcagtcctcagggagaacttttttgggaaccaggagctgggtctggacaaaggagtaccctgcattacgtgggatatgtgtgatcaattggggacatgcgacacacaatgagggtgtcatgacaatgcatgacacgtacggttatatgtggcagtgtgaccccttgacatgtggcgttacatgaaagtcagtgtggcacgtgttctgtggcatgggtgctggcatcccaagtagcaggatacatgattgttggtctatatatgacacatgacaaatgtccatgtcacaggactcatggctggccagatgacctcaggctggcccaagatctaatttattaatttttaaagcaaatacatatttatagattgtgtgtatggagcagctaagtcaggaaaagtcttccgcccgagctgggaggggagagtgtccatgcactgaccagtccaggggctcaagggccagggctctggaacaagccagggactcagccattaagtcccctcctgcctcaatcctcagcctacccatctataaacttgatgactcctcccttacttacatactagcttccaaggacaggtggaggtagggccagcctggcgggagtggagaagcccagtctgtcctatgtaagggacaaagccaggtctaatggtactgggtagggggcactgccaagacaataagctaggctactgggtccagctactactttggtgggattcaggtgagtctccatgcacttcacatgttacccagtgttcttgttacttccaaggagaaccaagaatggctctgtcacactcgaagccaggcttgatcaataaacacaatggtattccacgtc

SEQ ID NO: 11 - DNA, 5673 bp, Pullus
ctagggatccacacagagtgaggatgctgcatataggcacgcaccttgtgttggggtgttgctgtgtgctgaaactctgccactggtagaccacttctttggcaggatatggaggaggaagcacctttgtatttgcatatgctgctggattgcacttaggacgactgaagggaggctgaccacacagcaatgcagcccacactccagaacgatgacttcaacctggaaggagctgtagcaagcatccgcccccagctagtggagttcctcagccatcgtctcgactggctgctctctgcttcccagcacttcctccctacggctgtcctcagcggcttggcaggcgtcactgaccacagagagaaagtctctgtgctcctcgacctgctggagaaggctggccatgccacctggaaacagtttgcacagtgcctctgcatggagtgtgacctgcccctggaaatggagatactgctcatgagttctgcaggagaaggtaacttgactcagaagcaagaagctcagacagacgcaagtgaccagtcctctgtgccaagagggttgacgcgccgccgccgtagcagcagccccatcagtgataagggtgctaaacagaaacgcctggactcagctgaaaagtatcaacgtcttctcgttgactccatctgcaagagatatggaagcaggagagcagcaggagcagcggcacaagagccaacgcagcctctggctttcagccaagcctttgtcaacctcgttcttcggcagagcaaggcctcccgactgaaagagagaacagacaaaccccgggaggacgtgcccagtgcccccgagccagaagaatgtgtggatacagccgtgagagtgtcagatctctttggcagcgtggtccgatcgggtacaacgaaggtgatctttttgtttggtaaaccaggtacaggcaagaccatgctgatgcacaggatttgtcagaagtgggcggaaggtgtcttacatcagtttctcttcactttcctgtttgagttccggcagttgaatttactaaaaagaaaactgactctgaaggagcttttgtttgacctgtttcttcagccagaagacagccccgatgcagtgttccagcacctcctggaaaacgcccaacgtacactgatcatctttgatgggctggatgagtttgtgggtaacatggacgtgtcatcagcttctaaaggaagcctgaaccgtagctccagtatgtccatatctgaactctttgcagagctttgtcatgggaacctcctacctggctgcacggtgttggtcaccagccgccctaagagagtccctgacttcttgataaacacagtagatttgctggcagaagtttgggggtttgatcacgagaaggttgaggagtatgtcagccactattttcgtcatcattcattcaaagaacaagccatcgctcacctgaagaacaacaccaagctcctcagcatgtgccagatccctgccctgtgttacgttgtctgcatctgtttggagtatttgctccttaagcatcagacgagcgtggagcttccccagaccatgacacagttttatatcaaaatgcttctcatcttcataaacaaacagcagggggagcatgcaggtgatgaggaggctcagctgaacagtaacaagaaggccattttgggtctgtgtgatcttgcactgaaaggcctggaagctaagaagctcgtgttttacgttggagatattccagagcacgtgaaagagtttgcttccttgcacggtctgctgactgtctttgaggtgaagacaaacagcgctcaccccgagactggctatgcctttgtgcacttgagcctgcaggagttttttgcagccctgtgtctcatgataagcaagagtgtggacaagagccacctgaagaggaagttatctctgaagtcaaagtggacgttaaggaatgaagcaaagactgggttcatggagagctttcacatcttcctctcaggcttgtcatccaaggagtgtaggacgtttatcatgttgctagctgaacaaagtgaagcttgggtgcaggataagcaagatgcaatccttcagtccctcaagaaactggcagccactcagctgaccgggcccaaagtcattgagctctgccattgcacgtttgaaacgcaaggcctcgaagtagcccagcacattgggagcctgctgaatttcaagtacgagtttaaaaacttcagactgacacctctggacatgtcggctttggtttttgtcatcaactcgggccaggatgtgactcacttggattttgcagggtgtcccttagacacggactgtttggagatgctggccagctgtaggaatgtggagcatctgagctttcggagtaggagatttggtgatgactttgctgctgcgctttcaaagggcttaggagaaatggggagcctgaagaaactagaggtgacaggtggaagcatcacagctgcggggctgactgacatggtccaagctttgtcacactgcctgcagctcgaggaaataaacttgcaggacaatcggatacagaatccagacgtaaagacagtgatggagctgttttccagaatggaaaagctgaagaaaatagatctgagcaagaacagtctttccttgaatgctgttctccttttggcaaaggagttcatcgcgtgtcagaatgctgctgagctacacgtcaggaaggacactgtgataatatctttctcgggcccatctgggaaagttccaaggtctttggatttgaagcgggaacaaaacaaggaatgtgtgactccaactagacatctgaggttgtgcttgcaggctcgctgcttgtcttcacaacatgcaaaggagattgtgtccattctgcagagctgcccacacctctctgaagtagatttatcagataacaagcttggggatgaaggctgcagtttcctgctggaaaacctctcctggatttcaatttcaaagcagctgaacctttatcataacactgcagtcctgaggtttacagaagatggagaatttgcttcccctcctgctagtagggaagagcctttgtaccctactgatgaccagtgggatgacaaagaaaaccagacacctgttccctccaaaaaaataagattgacatactgtcgttttcaagccagtgacctggagaagctgtgtgcagtcctgaaggaatgtggcagcatctctgagctggagctaaataacaatcacctctccctgagctctgtgttctgcttagctcagtccttgtgtacactggaacacttggaaacaatggacctcagcttaggacgcatgcaggtggttcatctaacctttggggacagaatcaggctcagaaggaccagcagatggagaagttgtttcctggtgcatccgaagcacgtaacaaatggacagtgctttcgtctcaggaactgcacggtgggtcctgaggacgtgaccaggctctgccagatgctgatgcagtgtacccagctgacggaaatcgatctgtgtggaaatgcactgaatgaccaaagcattgagagattactgagcttcctgccacacttctgccagctgacactactgagtattaggaacaacaccttttccccatgttgtgctgtattatttgccaactcgatcaatctctgtgagcgcatcagaagggtggaagtccggtcaagtcagaatgcttttttacacttaagaacaagcacgcagggccagaagacatcctgcaggctgactgactgtgccatcggccaaaggcagatagagaagctctgcagagtcctggagcagcatgggtgtctggcagaactagatctctccaggaatcagctgggagatgaagtcttgaggttcctcttggaccacttgcatcgagtgcctgttacctgttctctcaatctcagccataataggatttctcagcgtggagttctgcatctcataaatacttttaccacttctggaaataccaccgaagtccaggtcagtctgtgctccaaggcaactttaatcatcaagatgacaagcagagatgacccaagaaagattttgaggctcacagagtgcagctttcagccagaacacttggaaaagctatgtttggttctggagaactgcaccaatctgacagagtgcatatcctccaacaacaacgtgacagtcgatgctgcaggggggcttttacgttcactgagtaaaactccaggtcctctaaaaatcagcatagaagagccctgggtatgtaagttgagtgtcacgagccttcttgagctggctgtccaggcccatggaaacatcactgcgatcatgatatgcaaagagaagaacctcttccaactaggtgtaaggcttccacactgcctggagaaggtgggatcagtcgttagcagattgaatttgaacgagccagaaatcaaacaggcttgcttctaccaaagggttcatgacaaatgcacccagcttcaggaactgagatggtctcacgttgaactccacgatgacacagaaatgctcgtcagtattttgctacctcttccagatctgaagaagtttgagctgacctcttgcagctttacaccaactggaattgattgcttgatcacaggcttgcagaggtgccaggccattgaagagctcaacctgggccacatgaagctcggcgatgctgccattcctaagctagtgtttggactctgtgaaatgccgtctctgaaaaggctgatcttgaaccataacagtattggtgacgatggctgctccagactcgcagaagccctgagcagcatgcactgcatggaagaaatcaatttaggccacaacaagattggagatctaggcctgataaatatagctgctgtcctgctggaaatgcaaaacttgaagagaatcgacctctcagggaactgtcccagtcctgctggaggagaaaagctgatggaagctcttgccaattgcaagcacctcgaggagctaattttatctgaaaacaaccttggggaaggaggtatccgtgccctgtccgaggggctgccgcgttttgagcacctccggaagattgagttgagactctgtggaatcactgatgatgcttcaaaattactctctcgtggcttccagcaatgccctgctgtggaggagatcatattgtcttggaacgcccttggtgatggaggagcccaagtgttggccagcatcctcccagggatggaaacgctgaagatgttagacctggagaaaaatctcattggtgcgtgtggtgctaccaggttggcagaggaacttgtcaggtgcccagaaattcagttcataaggctgtgggacaatccggtgcccaaaggcctcgctgaaagtttgacaagccaagacccaagactgtgcttctccttctgttagcgaaggacagccctcacctgatagaaattgctctccgtttcccattggcctcaagaattgcactaaaagggatcctgaagaacccaggaaaccaaaattcctccagttaaaacctgtaattatgtcttccttcattgcactggatgaaatgtgagcacttacgtgtgttttgtttatggtgagcttttattttgaaccaaaagactgaagaactgctgctgagcaggcagcattctc

SEQ ID NO: 12 - protein, 1915 aa, Mus musculus
MDAESIRLNNENLWAWLVRLLSKNPEWLSAKLRSFLPTMDLDCSYEPSNPEVIHRQLNRLFAQGMATWKSFINDLCFELDVPLDMEIPLVSIWGPRDEFSKQLGAGEESCPGPQLYHGAKRPFQSYGSSPRRKNSKKQQLELAKKYLKLLKTSAQQWHGGVCPGAWLTPHSPQTYIPPVLQWSRATAPLDAQEGATLGDPEAADNIDVSIQDLFSFKAHKGPRVTVLLGKAGMGKTTLAYRLRWRWAQGQLDRFQALFLFEFRQLNMITQLPTLPQLLFDLYLMPESEPDAVFQYLKENAQEVLLIFDGLDEALHADSVGTDNAGSALTLFSELCHGNLLPGCWVMTTSRPGKLPSCVPTEAATVHMWGFDGLRVEKYVTCFFSDLLSQELALKEMRTNARLRGMCAIPALCTVTCFCLRRLLPGSSPGQSAALLPTITQLYLQMVETFSPSETLLDTSILGFGKVALRGLDTGKVVFSVEDISPQLMSFGAVHSLLTSFCIHTRPGHEEIGYAFVHLSLQEFFAALYLMASHTVDKDTLVEYVTLNSHWVLRTKGRLGLSDHLPAFLAGLASHTCHMFLCQLAQQDRAWVGSRQAAVIQVLRKLASRKLTGPKMIELYHCVAETQDLELARFTAQSLPSRLSFHNFPLTHADLAALANILEHRDDPIHLDFDGCPLEPHCPEALVGCGQVENLSFKSRKCGDAFAEALCRSLPTMGSLKTLGLTGSRITAQGISHLIQTLPLCSQLEEVSLHDNQLKDPEVLSLVELLPSLPKLQKLDLSRNSFSRSILLSLVKVAITCPTVRKLQVRELDLIFYLSPVTETATQQSGASDVQGKDSLKEGQSRSLQLRLQKCQLRIRDAEALVELFQKSPQLEEVNLSGNHLEDDGCRLVAEAASQLHIAQKLDLSDNGLSQTGVTYVLKAMSTCGTLEDLHISLLNNTVVLTFAQEPREQEGSCKGRAPLISFVSPVTSELSQRSRRIRLTHCGFLAKHTETLCEALRASCQTHNLDHLDLSDNSLGGKGVILLTELLPGLGPLKSLNLSRNGLSMDAVFSLVQCLSSLQWVFHLDVSLESDCIFLRGAGTSRDALEPKFQTGVQVLELSQRYTSRSFCLQECQLEPTSLTFLCATLEKSPGPLEVQLSCKSLSDDSLKILLQCLPQLPQLSLLQLRHTVLSSRSPFLLADIFNLCPRVRKVTLRSLCHAVLHFDSNEEQEGVCCGFPGCSLSQEHMETLCCALSKCNALSQLDLTDNLLGDIGLRCLLECLPQLPISGWLDLSHNNISQEGILYLLETLPSYPNIQEVSVSLSSEQIFRMCFSKKEGAGTSLRLCECSFSPEQVSKLASSLSQAQQLTELWLTKCHLDLPQLTMLLNLVNRPTGLLGLRLEEPWVDSVSLPALMEVCAQASGCLTELSISEIQRKLWLQLEFPHQEGNSDSMALRLAHCDLETEHSHLMIQLVETYARLQQLSLSQVSFNDNDGTSSKLLQNILLSSCELKSFRLTFSQVSTKSLTHLAFGLGHCHHLEELDFSNNSLREEDTELLMGALQGTCRLKKLHLSFLPLGASSLALLIQGLSRMTLLQDLCLSHNQIGDVGTQCLAAILPKLPELRKFDLSHNQIGDVGTQCLAAILPKLPELRKFNLSHNQIGHVGTQCLAAILPKLPELRKFDLSRNQIGDVGTQCLAAILPKLPELRKFDLSGNRIGPAGGVQLVKSLTHFEHLEEIKLGNNALGEPTALELAQRLPPQLRVLCLPSSHLGPEGALGLAQALEQCPHIEEVSLAENNLAGGVPRFSKRLPLLRQIDLEFCKIEDQAARHLAANLTLFPALEKLLLSGNLLGDEVAAELAQVLPQMGQLKKVNLEWNRITARGAQLLAQGLVQGSCVPVIRLWNNPILNDVAQSLQSQEPRLDFSITDQQTL

SEQ ID NO: 13 - DNA, 6945 bp, Mus musculus
atggacgctgagagcatccgactgaacaatgaaaacctgtgggcctggcttgtgaggctgctgagcaaaaacccagaatggctgagtgccaagcttaggtccttcctcccgaccatggacctggactgcagctatgagccctccaaccctgaggtcatccaccgtcagctcaacaggctgttcgcccaaggcatggccacctggaagtctttcatcaatgatctgtgcttcgagcttgatgtacctttggacatggagataccactagtgagcatctggggcccaagagatgaattctctaagcagctaggggcaggagaggagagctgccctggacctcagctgtaccatggtgccaaacgtccttttcagagctatgggtcctcaccccgtcggaagaacagcaagaagcagcagctcgagttggccaagaagtatctgaagctcctgaagacctctgcccaacagtggcatggtggtgtgtgcccaggagcatggcttactcctcactccccacagacctacatccccccagttcttcagtggagccgggctacagcacccttggatgctcaggagggagccactttaggagaccctgaagcagccgacaacattgatgtgagcatccaggatctcttcagcttcaaggctcacaagggacctcgagtgacagtactactgggaaaggctgggatgggtaagaccactctggcatatcggctccgctggaggtgggctcaaggtcagctggaccgcttccaggctctgttcctgtttgagttccgccagctcaacatgatcacacagttgccgaccctgccccagctcctctttgatctgtacttgatgccagagtcagagcctgatgctgtttttcagtacctaaaggagaatgctcaggaagtcctactgatctttgatgggttggatgaggctctccatgctgattctgtgggtactgacaatgcaggctcggccctcaccctcttttctgaactctgccatgggaacctcctgccaggctgctgggtgatgaccacctcccgtcctgggaaactgccttcctgtgtgcccacagaagcagccacggtacacatgtggggctttgatgggctacgagtagagaagtatgttacctgcttcttcagtgatcttctgtctcaggaattggccttgaaggaaatgagaaccaatgcacgtctccggggtatgtgtgcaatacctgcgttgtgcacagtcacctgcttctgccttcgccgtctgcttcctggaagctccccagggcagtctgcagccctcctgcccaccataacccagttgtacctgcagatggtggaaacttttagccccagtgagactctgcttgacacatccattttgggctttggaaaggtggcccttaggggactggacactgggaaagtcgtcttctctgtggaagacatctctccccaactgatgtcttttggagctgtccacagcctcctgacctccttctgtatccacacccgccctgggcatgaagaaataggctatgcttttgtccaccttagcctgcaggagttctttgctgctctgtatctgatggccagtcacacagtggacaaagacacactcgtcgagtatgtcaccctcaattcccactgggtgctgaggaccaaaggtagactgggcctctcggatcatctccctgccttcctggcaggcctagcctcccacacctgccacatgttcctctgccaactggctcagcaagacagagcctgggtgggctccaggcaggccgctgtgatccaggtgctaaggaagttggctagccggaaactcacagggccaaagatgatagagttgtatcactgtgtggctgagacccaggatctggagctggcgaggttcactgcccaaagccttccttcccgcctctccttccacaatttccctctgacccatgcagacctggctgcactggctaacattttggagcacagggatgatcccattcacctggattttgacggctgcccactggagccccactgcccagaggctctggtaggatgtgggcaggtggagaatctcagctttaaaagcagaaagtgtggggatgcctttgcagaagccctctgcaggagtttgcctactatggggagcctgaagacgctggggttaacaggaagtaggattacagcccaaggcatcagccacctgatacagacattgcccctctgttcccagctggaggaggtcagtttgcatgacaaccagctcaaggacccagaggtgctgagtcttgtggagctgcttccctccctgccgaagctccagaaacttgacctgagccgaaacagcttctccaggtcaatcctcctgtctttggtgaaagtggctatcacatgtcctactgtcaggaagctgcaggtcagggaattggacctcatcttctatctgtccccagtcacagagacagctacacagcaatcaggagcatcagatgtacagggcaaagacagcctgaaggaaggacagagtcgaagcctgcaactcaggcttcagaaatgtcagctcaggatccgtgatgcagaagcccttgtggagctattccagaaaagcccccagctggaggaagtgaatctttcagggaaccatttggaagatgatggctgtcgacttgtggcagaggctgcttcacagcttcacattgcccagaaactggacctcagcgacaatggcctctctcagactggggtgacctatgtgttgaaagccatgagcacatgtgggaccctggaagacctgcacatcagcctgctaaacaacaccgtggtactcacatttgcccaggagccgagggagcaggaggggagctgcaaggggagggcaccacttatcagcttcgtgtctcctgtgacctccgagttgtcccagagatctcgaaggatcaggctgacacactgtggcttcctggccaagcacacagagacgctctgtgaggcactgagagccagctgccagacccacaacctggatcatcttgacctatcagacaactctttggggggcaaaggagtgatcctgctaaccgagctgctcccaggactgggtcccctgaagtccttgaacctcagcaggaatggcttgtccatggatgctgtctttagcttggtccagtgtctgtcttccctgcagtgggtgttccacttagatgtcagccttgagagtgactgcatcttccttcgaggagctgggaccagcagggatgcactagaaccaaaattccagactggagtccaggtactggagctgagtcagcggtacacctcccggagcttctgccttcaagagtgtcagttggaacccacgagccttaccttcctctgtgccactctggagaagagcccagggcctctggaagtccaattgtcctgcaagtccctgagtgatgacagcctgaagatcctgctgcagtgcctgccccagctgcctcagctcagcctgctgcagctgagacacacagtcctatcttcaagaagccccttcctgctggctgacatcttcaacctgtgcccacgggttcggaaggtcactctcaggtccctatgtcatgctgttctgcactttgactccaatgaggaacaagagggtgtgtgctgtggcttcccgggatgcagcctcagccaggagcatatggaaacactgtgctgtgcactgagcaagtgcaatgccctcagccagcttgatctcacagacaatctgctgggtgacatcgggctcaggtgccttctggagtgcctgccacagctgcccatctctgggtggcttgacctcagtcacaacaatatctctcaggaagggatcctgtacctgctggagactctgccttcctaccctaatatccaagaggtctctgtgagtctgagctctgagcagatcttccgaatgtgcttctccaaaaaggaaggagccgggacatcgctgaggctatgtgaatgcagcttcagcccagaacaagtatccaaattggcttccagcctgagccaggcccaacagctcacagagctctggctgaccaagtgtcacttggacctgccacagctgaccatgctcctgaacctggtgaatcggccaacagggttgctgggcctcaggttggaagagccctgggtggactctgtcagcttaccagctctaatggaagtctgtgcccaggcctcaggatgtctcactgagttaagcatctctgagatccaaaggaagttgtggctccagctggaatttcctcaccaagaaggcaattctgattccatggctctcaggttggctcactgtgacctggagaccgagcacagccatcttatgatacagttggtggagacatatgccagactgcagcagctgagcttgtctcaggttagcttcaatgacaatgatggcaccagctccaagctgctgcagaacatcctgctatcaagctgtgagctgaagagcttcaggctgaccttcagccaagtgagcacaaagagcctcacccatctggcatttggtctgggacactgccaccatctggaagagctggacttctctaacaacagtctccgtgaggaggacacagagctactgatgggagcccttcagggaacctgcagactgaagaagctgcaccttagtttcttaccattgggagcctcctctctggccttgctcattcaaggactaagccgaatgactctactacaggacctttgcttgagtcacaaccagatcggagatgtgggtacccagtgcttagctgccatcttgccaaaactgccagaactcaggaaatttgacttgagtcacaaccagatcggagatgtgggtacccagtgcttagctgccatcttgccaaaacttccagaactcaggaaatttaacttgagtcacaaccagatcggacatgtgggtacccagtgcttagctgccatcttgccaaaacttccagaactcaggaaatttgacttgagtcgcaaccagatcggagatgtgggtacccagtgcttagctgccatcttgccaaaacttccagaactcaggaagtttgacctctcagggaacaggattggcccagctgggggagtgcagctggtgaagtctctcacccatttcgagcacttggaggagatcaaactgggcaacaatgctctgggggagcccacagccctggagctggctcagagattgcccccacaactcagggtcctgtgcctgccatccagtcatctgggtccagagggggccctgggcctggcccaggccctggagcaatgcccacacatagaagaggtcagcttggcagagaataacctggctggaggtgtcccacgtttctccaagaggctcccactgcttagacaaattgacctggagttctgcaagattgaagaccaagctgccaggcaccttgctgccaacctcacactcttcccagccctggagaaactcttgctgtccgggaatctccttggagatgaggtggctgctgagttggcccaggtcctgccgcagatgggacagctgaagaaagtgaacttggagtggaaccggatcacagcacgtggagcccaactcctggctcaagggctggtccagggatcctgcgtcccagtcattcgcctctggaacaaccccattcttaacgatgtggcccagagtctgcagagccaggaacccaggctggacttctccatcactgaccagcagactctttgaggaacccaacggacttctcaagacttatgagtccagtccagtgacagccacatcacccaccaggagagggggctcagaagaacctctgttggtgcccagcatcctacgaatgccccaggaggaaagaacacatttttcctgtcacgatccctagggagcgcattcttagggactgagagctttgtttgaccaaaagagggccatatatcatatatcatacatgacatatgttaccaatggggcattgacattatgagagtattatgaggttatatatacaggaaaggctgtgtatggtgcactatgaccccttgacatgggaaggtcatactcagagtaggtcaaatgtagaattacatggaacttgtggcatgacacctggtcatagcctcttagcactgtaattggaaatacacatgatcaggaatccacttatgacatgatgtccatatcacatgacacacaacttgacaggggaccacagataagtccaagacctaatttattattattttaaaaaacatgaagttaggctaatggtataggctatgttatatgaacagccttttagctgttcaggaatctgaggcagacagatcgcaagttcaaggtcaatctaggtaatttagtaagatcctgtctcaaaaggtcagaaagggctggggatatggctcaatgctagagtgcttcctagcatgtgtaaagccctaagctcaatgctcggtgctttttttttttttaagtatttataagcaccttctggagaaactttttgtcttgtgctaggagagggagttaagaattgagtggcccaatggtccggggtctgggacatgccaaggacttagcatgaagtcccctcctgccagagcctcaagtctctcttctgttaatcgtgttcttccaaggacttccagcccaggcttaggagttaacctgaccttgggagagttcgcactcatctcttctttcctggtttaagggacaaagccgagctacaggtttagggataagtacttccaggataataggcaagaccactaggtctagcctacccactacctcagcaggattcagaaaccacttgaaactccatggtgttttgctttgttttgcaacttccaaaggctaaactcacaacactccatatcaaatgatcaataaacgcagatgtaactggtgtctgatgtaaacgcc